(19) United States
(12) Patent Application Publication
(10) Pub. No.: US 2022/0151463 A1
(43) Pub. Date: May 19, 2022

(54) ENDOSCOPE HANDLE

(71) Applicant: AdaptivEndo LLC, Louisville, KY (US)

(72) Inventors: Hershel E. Fancher, Georgetown, IN (US); Gregory R. Furnish, Louisville, KY (US); Galen R. Powers, Louisville, KY (US); Mark A. Griffin, Louisville, KY (US); Trevor E. Powers, Louisville, KY (US); Nicholas Canfield, Louisville, KY (US); Michael W. Goldenbogen, Xenia, OH (US); Summer E. Adams, Louisville, KY (US); Christopher J. Gostout, Rochester, MN (US)

(21) Appl. No.: 17/650,008

(22) Filed: Feb. 4, 2022

Related U.S. Application Data
(63) Continuation of application No. PCT/US2020/045004, filed on Aug. 5, 2020.
(60) Provisional application No. 62/883,080, filed on Aug. 5, 2019.
(60) Provisional application No. 62/958,948, filed on Jan. 9, 2020.
(60) Provisional application No. 62/959,198, filed on Jan. 10, 2020.
(60) Provisional application No. 62/959,346, filed on Jan. 10, 2020.
(60) Provisional application No. 62/959,370, filed on Jan. 10, 2020.
(60) Provisional application No. 62/959,387, filed on Jan. 10, 2020.
(60) Provisional application No. 62/958,744, filed on Jan. 9, 2020.

(51) Int. Cl. A61B 1/005; A61B 1/00; A61B 1/008

(52) U.S. Cl. CPC A61B 1/0052; A61B 1/00042; A61B 1/00147; A61B 1/00103; A61B 1/008

(57) ABSTRACT

An endoscopic system comprising an endoscope having a reusable hand-piece and separable single-use shaft assembly, a single-use cartridge and a console. The single-use shaft assembly includes fluid paths and electrical connectors configured to extend through the single-use cartridge. The single-use cartridge comprises a housing defining fluid paths and electrical connectors. The reusable hand-piece comprises articulation control(s) for articulation of the single-use shaft assembly.

### CROSS-REFERENCE TO RELATED APPLICATION

This application claims the benefit of U.S. Provisional Application No. 62/883,080 filed Aug. 5, 2019, U.S. Provisional Application No. 62/958,948 filed Jan. 9, 2020, U.S. Provisional Application No. 62/958,744 filed Jan. 9, 2020, U.S. Provisional Application No. 62/959,370 filed Jan. 10, 2020, U.S. Provisional Application No. 62/959,387 filed Jan. 10, 2020, U.S. Provisional Application No. 62/959,198 filed Jan. 10, 2020, and U.S. Provisional Application No. 62/959,346 filed Jan. 10, 2020, which are hereby incorporated by reference.

### BACKGROUND

This invention generally relates to endoscopes, including endoscopes used in gastroenterological applications.

Endoscopes are used in a wide variety of medical procedures to visualize internal cavities or potential spaces within the human body during either diagnostic or therapeutic procedures.

One factor affecting endoscope design and use is infectious disease transmission. Although infectious disease transmission has always been a risk with reusable endoscopes, this risk has increased with the advent of antibiotic resistant bacteria. Once transmission of these bacteria has occurred between patients, antibiotic resistance makes it difficult to treat these infections. Many of these bacteria, including staphylococcus, have the ability to form a protective outer barrier, frequently referred to as a bio-film, which protects the infectious bacteria from during cleaning procedures.

Another recent development that increases the risk of infectious disease transmission is an ever-increasing number of diagnostic procedures that require the use of smaller and more complicated instruments and longer working lumens and distal manipulators, e.g. elevators, associated with the endoscope distal articulating shaft. Together, the increasing prevalence of difficult to treat infectious diseases and more complex, difficult-to-clean endoscopes make the sterilization and re-use of endoscopes an increasingly risky activity.

Reducing or limiting the potential for infectious disease transmission is desirable. Accordingly, new endoscope designs are desired. Thus, there is a need for improvement in this field.

### SUMMARY

The present disclosure pertains generally to endoscopes. In certain aspects, the present disclosure pertains to endoscopes having a reusable hand-piece removably coupled to a single-use shaft assembly. The reusable hand-piece includes articulation controls, and the single-use shaft assembly includes an articulation wire articulating assembly that engages the articulation controls when the reusable hand-piece and single-use shaft assembly are coupled to one another (e.g., when the housing of the reusable hand-piece and a housing of the single-use shaft assembly are coupled together).

The reusable hand-piece may have an optical and/or electrical connector that engages to with a corresponding optical and/or electrical connector of the single-use shaft assembly when the reusable hand-piece is coupled to the single-use shaft assembly (e.g., when the housing of the reusable hand-piece and a housing of the single-use shaft assembly are coupled together). The optical and/or electrical connector of the reusable hand-piece may be part of an electronics module. The electronics module may include imaging controls.

The single-use shaft assembly can include an imaging device (e.g., image sensor such as CCD or CMOS sensor or a lens) and/or a light emitter (e.g., LED or a fiber optic cable). The imaging device and/or light emitter can be positioned at a distal end of the single-use shaft assembly. The optical and/or electrical connectors can provide communication between the electronics module of the reusable hand-piece and the imaging device and/or the light emitter of the single-use shaft assembly.

The light emitter may include an element that produces light (e.g., an LED) or may communicate light provided proximally of the articulating distal portion. For example, the light emitter may be a light pipe (e.g., fiber optic cable). The imaging device may include a sensor that converts light into electrical signals (e.g., a CCD or a CMOS sensor) or a lens arranged to pass light from the articulating distal portion towards the proximal portion of the shaft.

The reusable hand-piece can include controls for controlling fluid flow through the single-use shaft assembly. Those controls can communicate with valves in the single-use shaft assembly and/or with external support equipment (e.g., pumps and/or valves in support equipment).

The endoscope assembly can include a lock that retains the reusable hand-piece and the single-use shaft assembly together in the assembled configuration. The lock can include a first portion on the reusable hand-piece and a second portion on the single-use shaft assembly, wherein the first and second portions of the lock cooperate to hold the reusable hand-piece and the single-use shaft assembly together in the assembled configuration.

The lock is actuatable to engage and/or disengage from the single-use shaft assembly. The lock can include a latch having catch mounted on a pivoting and/or deflectable latch arm. The latch can be biased with a biasing member (e.g., spring) into a latching configuration that can hold the reusable hand-piece and single-use shaft assembly together. The lock may include one or more magnets that hold the reusable hand-piece to the single-use shaft assembly when in the assembly configuration. The lock can be arranged for actuation (e.g., to engage and/or disengage) without use of a hand tool (e.g., screwdriver). Preferably, the lock is actuatable with fingers. The lock can be defined by a portion of the housing of the reusable hand-piece and/or the single-use shaft assembly. For example, the housing may define a deflectable tab and/or a living hinge for the lock.

The lock can be arranged to indicate when the reusable hand-piece and the single-use shaft assembly are locked to one another. For example, the lock can provide an audible “click” when the reusable hand-piece and the single-use shaft assembly are locked to one another.

The single-use shaft assembly can include one or more ports communicating with fluid flow paths (e.g., lumens) of a shaft of the single-use shaft assembly. The fluid flow paths may extend along a length of the shaft to one or more openings in at the distal end of the shaft. The fluid flow paths may be arranged for irrigation, insufflation, aspiration, and/or for receipt of a surgical tool (e.g., forceps, a cutter, and/or a ligation device). The reusable hand-piece may be absent of fluid flow paths and/or fluid connectors in fluid communication with fluid flow paths of the shaft.

The articulation controls of the reusable hand-piece can include articulation knobs. The articulation knobs can be arranged to receive articulation input from the user (e.g., in the form of rotational movement). The articulation knobs can communication with cams and/or gears of the separable single-use shaft to actuate control wires of the single-use shaft assembly. The user controlled articulation knobs can include two knobs having and/or rigidly attached to concentric articulation shafts.

The concentric articulation shafts each have a pulley/pinion engaging portion. This portion may have a cross-sectional geometric shape capable of transmitting torque. Preferably the geometric shape has a geometry that interferes with a geometry of the pulley/pinion so rotation of the articulation shaft rotates the pulley/pinion. This geometric shape can include, but is not limited to, oval, spline, square, or star, just to name a few non-limiting examples.

Advantageously, the concentric articulation shaft configuration permits attachment of the single-use shaft assembly to the reusable hand-piece without the need to orient the articulation knobs or the distal articulating tip to obtain a neutral reference position. Regardless of the orientation of the single-use shaft assembly (coiled or straight) at the time of attachment, the articulation knobs can freely rotate while the distal shaft (insertion tube) is manipulated prior to use by the clinician—assuming no brake of the articulation knob and brake assembly is applied.

The single-use shaft assembly can include one or more pulley assemblies. The pulley assemblies include at least one pulley arranged to receive an end of an articulation shaft. Rotation of the articulation knob associated with the articulation shaft rotates the at least one pulley. Associated with the at least one pulley is an articulation wire or a pair of articulation wires (e.g., two separate wires or one continuous wire looped over the pulley and having wire segments extending from opposing sides of the pulley) configured to bend a distal shaft section of the single-use shaft assembly upon actuation. The articulation wires may be fixed (e.g., tied) to the pulley and/or extend around at least a portion of the pulley (e.g., loop around the pulley). Preferably, the one or more pulley assemblies include two pulleys (one for each articulation shaft) and articulation wires (at least one for each pulley). Preferably the articulation wires are configured to bend the distal shaft section in orthogonal planes.

The single-use shaft assembly can include one or more rack and pinion assemblies contained within a rigid housing. For example, the single-use shaft assembly may have a proximal rigid housing having a surface that supports and positions the rack and pinion assembly of the single-use shaft assembly to receive the pinion engaging portions of the articulation shafts.

Each pinion contained within the proximal rigid housing associated with the separable single-use shaft possess a circumferential series of teeth that engage a set of opposed racks. Each set of opposed racks is associated with a pair of opposing articulation wires. Each articulation wire can be rigidly fixed at one end to a rack and on the other end to the distal tip of the articulating section of the shaft. Rotation of the pinion, such as by means of a user input torque applied to the corresponding knob, results in a tensile force applied to the articulating distal section.

A preferred embodiment utilizes two pinions, each associated with a pair of racks and articulation wires. Each pair of racks and articulation wires provides means for articulating the distal shaft section in a single plane. Two pairs of articulation wires provide distal shaft articulation in two different plans that can be perpendicular to one another, with a longitudinal axis of the proximal shaft portion passing through the intersection of the planes.

The preferred embodiment incorporates either a single or a pair of pulleys incorporating the geometric engagement shape of an articulation shaft and a pair of opposed steering wires for each pulley.

The mating features of the concentric articulation shafts associated with the reusable hand-piece and the mating features of the pulleys/pinions associated with the separable, single-use shaft assembly are suitable for transmitting force in the form of torque applied to the articulation knobs to tensile force in the articulation wire(s) and corresponding movement of the distal shaft articulating section. Preferably the mating features do not rely solely on friction between the articulation shaft and pulley/pinion. Preferably the mating features include a geometric interference between the articulation shaft and pulley/pinion.

A portion of the articulation shafts, such as the pulley/pinion engaging portions, may be tapered along a length of the articulation shafts. For example, the articulation shaft may have a portion taper from small to large in a direction towards the articulation knob. Advantageously, such an arrangement can aid in mating the articulation shafts with the pulleys/pinions of the single-use shaft assembly when the reusable hand-piece is coupled to the single-use shaft assembly.

The reusable hand-piece can include one or more brakes that apply an adjustable level of rotational resistance to the articulation control (e.g., articulation control knobs). A separate brake mechanism can exist for each articulation control knob and its articulation wire or associated pair of articulation wires for applications where two plane articulation is desired. Furthermore, the brake mechanism(s) may be consolidated within the envelope defined by the knobs, thereby creating space within the reusable hand-piece housing for the single-use shaft assembly. Furthermore, the brake mechanism(s) may not utilize a fixed method for applying force against the friction surfaces and/or brake material, such as rotation of a threaded shaft. Preferably the brake mechanism(s) utilize(s) a spring element to apply force between the friction surfaces and/or brake material, negating the need to adjust or calibrate the braking force. Preferably the brake is disengaged by compressing the spring element to remove the force from the friction surfaces and/or brake material.

In arrangements disclosed herein, the articulation controls and an electronics module that can incorporate control switches and/or navigation switches are preferably mounted to the housing of the reusable hand-piece. The articulation wire actuating assembly and connector of the single-use shaft assembly are preferably mounted to the housing of the single-use shaft assembly. Accordingly, separation of the housing of the reusable hand-piece from the housing of the single-use shaft assembly can separate the articulation controls from the articulation wire actuating assembly and the electronics control switches and/or navigation switches from the connector of the single-use shaft assembly.

A preferred embodiment includes an electronics module that can include a plurality of switches mounted to the housing of the reusable hand-piece that is in electrical communication with the wire harness associated with the single-use shaft assembly via an electrical connector. The plurality of switches may further incorporate a unique resistance value associated with each switch and each switch connected in parallel to a pair of electrical conductors. This configuration can minimize the number of connectors required to communicate the specific switch or combination of switches that are depressed by the user. Additionally or alternatively, the electronics module of this embodiment may further incorporate a microprocessor to minimize the number of connectors required to communicate the specific switch or combination of switches that are depressed by the user.

Advantageously, the reusable hand-piece of the endoscope assembly can remain entirely outside of the body of a patient during an endoscopic procedure while the single-use shaft assembly has a portion positioned within the body of the patient during the endoscopic procedure. Accordingly, the reusable hand-piece and single-use shaft assembly can be separated from one another after the procedure and the single-use hand-piece discarded (or reprocessed). As there are no fluid lumens of the reusable hand-piece that must be cleaned and sterilized, the cleaning (aka “reprocessing”) effort between procedures using the reusable hand-piece is dramatically reduced. Additionally, as no portion of the reusable hand-piece, which is used for multiple patients, is inserted into the patient, the risk of infectious disease transmission can be dramatically reduced.

Single-use shaft assemblies can be arranged and/or provided in a variety of configurations to support upper and lower endoscopies. For example, single-use shaft assemblies may be arranged for colonoscope, gastroscope, sigmoidoscope, and/or duodenoscope procedures, just to name a few non-limiting examples. Additionally or alternatively, single-use shaft assemblies can be provided in various specialty configurations, e.g. pediatric insertion tube diameters. Advantageously, the ability to use the same reusable hand-piece for a variety of single-use shaft assemblies and/or procedures can substantially reduce capital investments by clinicians, clinician groups, and/or medical centers by eliminating the need to stock a plurality of dedicated scopes for each type of procedure, e.g. colonoscope, gastroscope, sigmoidoscope, duodenoscope, etc.

Methods of assembling an endoscope assembly, disassembling an endoscope assembly, and/or using an endoscope assembly are envisioned. Such methods can comprise connecting a housing of a reusable hand-piece to a housing of a single-use shaft assembly, wherein the reusable hand-piece has articulation controls and an electronics module and the single-use shaft assembly has an articulation wire actuating assembly and a connector; and wherein the connecting connects the articulation controls to the articulation wire actuating assembly and the electronics module to the connector of the single-use shaft assembly. Additionally, or alternatively, methods can comprise separating the housing of the reusable hand-piece from the housing of the single-use shaft assembly to separate the articulation controls from the articulation wire actuating assembly and the electronics module from the connector of the single-use shaft assembly. The methods can include providing a portion, or all, of any endoscope assembly described herein.

Advantageously, the systems, assemblies, devices, and methods disclosed herein can increase the quantity of endoscopy procedures that can be performed by a clinician and/or facility in a day by reducing and/or eliminating the time delays associated with existing reusable scopes that must undergo extensive reprocessing procedures (i.e., cleaning) between uses. By using a single-use shaft, it is no longer necessary for the clinician and/or facility to reprocess (i.e., clean) the shaft and lumens of the shaft. Now the clinician and/or facility may simply wipe down the reusable hand-piece and connect a new, sterilized, single-use shaft assembly to the reusable hand-piece to prepare the endoscope assembly for another procedure.

Advantageously, the systems, assemblies, devices, and methods disclosed herein can allow a clinician to perform multiple and/or various procedures even without dedicated, reusable scopes and associated reprocessing equipment, supplies, and clean water. This can be particularly advantageous in battlefield settings or remote clinics with limited resources. In these cases, the reprocessing equipment, reprocessing supplies, trained reprocessing personnel, and reprocessing laboratory setting may not be available. Advantageously, the endoscopes disclosed herein can be prepared for a new procedure by simply using a wipe and antiseptic solution to clean the exterior surfaces of the reusable hand-piece and connecting a new single-use shaft assembly.

Advantageously, the reusable hand-piece can provide user-familiar-features using higher precision reliable components associated with the articulation knobs and the clutch.

Advantageously, incorporating the articulation handles and clutch into the reusable hand-piece reduces the components of the separable single-use shaft assembly thus resulting in a lower cost single-use portion of the endoscope.

The proximal rigid housing associated with the separable single-use shaft assembly can also include features for managing lumens for tool, irrigation, and aspiration. These features may either secure fabricated connectors or incorporate features for connecting to external devices and tubing. Advantageously, incorporating these lumen and connector features into the proximal rigid housing of the separable single-use shaft can reduce and/or prevent infectious disease transmission by segregating potential bodily fluid contact surfaces to the separable single-use shaft assembly.

The reusable hand-piece can include a control module, switches, and electrical connector while the single-use shaft assembly can include an optical sensor, optical sensor module, and electrical connector. A set of conductors can transmit both optical and control data back to a console from the assembled endoscope.

Advantageously, the incorporation of an electronics module and switches facilitates transmission of control data while simplifying the components associated with the separable single-use shaft.

Advantageously, the transmission of image data and associated metadata from the assembled endoscope to the console facilitates patient and image data storage and sharing

The reusable hand-piece can include an electronics module (e.g., circuit board) arranged to control at least a portion of support equipment (e.g., one or more pumps and/or valves in support equipment). One or more switches of the re-usable hand-piece can be associated with the electronics module to control the support equipment. Advantageously, such an arrangement can eliminate the need for valves for the irrigation and aspiration lumens to be physically associated with the reusable hand-piece and/or the separable distal shaft assembly.

The arrangements disclosed herein can utilize low cost, miniature high-resolution cameras. Advantageously, the low cost of the components can allow for single-use endoscopes that satisfy cost, dimensional, and resolutions requirements set forth by health care providers and/or insurers.

The single-use shaft assemblies disclosed herein can be intended for one-time-use. Advantageously, a single-use medical device can reduce transmission of infectious diseases.

Applicant has also observed that expertise associated with the assessment of particular diseases and the performance of novel therapeutic procedures has become increasingly concentrated at clinical research institutions or larger healthcare facilities. Advantageously, the endoscopes disclosed herein can facilitate the dissemination of patient and image data.

The present disclosure further pertains generally to endoscopes. In certain aspects the present disclosure pertains to single-use endoscopes and single-use cartridges attached hereto. The endoscope may include multiple fluid paths and one or more electrical conductors extending to the cartridge. The cartridge, in turn, may connect some or all of the fluid paths and/or one or more electrical conductors to the control console to allow the passage of fluid (e.g., liquid or gas) and/or power and/or data between the endoscope and the control console.

The endoscope may include a first fluid path, a second fluid path, a third fluid path and/or an electrical conductor. The first fluid path, second fluid path, and/or third fluid path may have portions defined by the cartridge. The cartridge can include a housing. The cartridge may further include a first electrical connector associated with the electrical conductor of the endoscope and a second electrical connector and configured to electrically connect the electrical conductor of the endoscope to an electrical conductor of a console.

At one fluid path of the cartridge (e.g., the first fluid path) can include a valve portion. The valve portion can align with a first actuator of the console, and the first actuator be actuatable to selectively close or open the valve portion of the fluid path.

The present disclosure further discloses cartridges having a housing having one or more inner surfaces defining one or more windows. Valve portions of one or more fluid paths may be positioned within the one or more windows. The one or more windows may be configured to receive one or more actuators from the console configured to close and/or open the one or more fluid paths.

The valve portions may include a flexible membrane positioned within a window defined by the housing. The flexible membrane may be tubing walls of a tube defining the one or more fluid paths.

The endoscopic system may utilize a control console to assist in operation of the endoscope. The console may include and/or be connected to a video monitor. The console may further include and/or be connected to a user interface for inputting commands The console can further include and/or be connected to a valve control assembly for receiving the cartridges. The valve control assembly can include a door to secure the cartridge. The door may include a latch that can be inserted into a keeper to provide the cartridge with compression. The compression helps to facilitate the interaction between the electrical connectors of the cartridge and the electrical connector of the console. The latch may be a lever latch.

The endoscope (e.g., the reusable hand-piece and/or the single-use portion) and cartridge can be sterilely sealed within medical packaging, such as a sterile medical tray. Preferably, the endoscope sealed within medical packaging is a single-use shaft assembly portion of an endoscopic system including a reusable hand-piece assembly and a single-use shaft assembly.

In addition to a single fluid path, the endoscopic system may include a second fluid path. The second fluid path may include second valve portion defined by the cartridge. The second valve portion may align with a second actuator of the console actuatable to selectively close and/or open the second valve portion of the second fluid path.

A fluid path of the cartridge (e.g., a third fluid path) may have a main portion and a branch portion. The main portion of the third fluid path of the cartridge can include a third valve portion. The third valve portion can align with a third actuator of the console actuatable to selectively close and/or open the third valve portion.

The branch portion of the fluid path of the cartridge may include a fourth valve portion. The fourth valve portion may align with a fourth actuator of the console actuatable to selectively close and/or open the fourth valve portion.

The endoscopic system may include a fluid path (e.g., a fourth fluid path) having a length extending between the endoscope and the console when the cartridge is connected to the console, the length being free of a valve.

The endoscope of the endoscopic system may include a reusable hand-piece assembly and a single-use shaft assembly, such as any of those disclosed herein. Preferably, the reusable hand-piece assembly may be selectively attachable to and detachable from the single-use shaft assembly. Preferably, the reusable hand-piece assembly supports an articulation control and when the reusable hand-piece assembly is attached to the single-use shaft assembly the articulation control engages a portion of the single-use shaft assembly for manipulation of a distal part of the single-use shaft assembly.

Preferably, at least one fluid path of the endoscope extends continuously from the endoscope to the cartridge to create a continuous fluid path and is free of a flow controlling valve. This continuous fluid path may have a length extending at least from the distal tip of the endoscope to the cartridge.

Fluid paths of the endoscope (e.g., the first fluid path, second fluid path, third fluid path, and/or fourth fluid path) may each be defined by a continuous flexible tubing.

A method of using the endoscopic system may include connecting the cartridge a console. Connecting the cartridge to the console can include connecting an electrical connector of the cartridge with the console to place an electrical conductor of the endoscope in electrical communication with an electrical conductor of the console to allow power and/or data to pass between the console and the endoscope. The connecting may also include positioning one or more valve portions of one or more fluid paths of the endoscope over one or more actuators of the console so the actuators may be selectively actuated to open and/or close the one or more fluid paths. Preferably, the connecting the electrical conductor and positioning the one or more valve portions occur simultaneously.

Advantageously, disclosed arrangements can reduce the number of separate connections made during setup by a user.

Advantageously, disclosed arrangements can reduce the number of leak points associated with endoscope lumens.

Advantageously, disclosed arrangements can reduce cost associated with the cleaning and reused of endoscope systems.

Advantageously, disclosed arrangements can reduce the risk of exposure to fluids that are a biohazard by replacing and/or eliminating multipart valves.

Advantageously, disclosed arrangements can allow the tracking and management of single-use endoscopes by the interaction of the console and the control module containing the unique identifier data.

In another example, a doorless cartridge and control valve assembly may be utilized. The control valve assembly may include a ledge surface and a latch. The cartridge may include a ledge surface and a latch receiving portion. In alternate examples, the control valve assembly may include the latch receiving portion and the cartridge may include the latch. When the cartridge is received by the control valve assembly the latch contacts the latch receiving portion to secure the cartridge to the control valve assembly. Separately the ledge surface of the cartridge and the ledge surface of the control valve assembly contact each other to secure and end of the cartridge to the control valve assembly.

The cartridge may further include an anvil similar to the anvil discussed in the control valve assembly with a door example above. The anvil is configured to provide counter force to the actuators when selectively closing fluid paths of the cartridge. In one example, the anvils may be located in the windows defined in the cartridge.

The endoscope assembly may further pertain to a system including a console with multiple control valve assemblies each configured to receive an independent cartridge. Each cartridge may be connected to an endoscope. Each endoscope may be a different type of endoscope. In some examples, a first endoscope is a duodenoscope and a second endoscope is a cholangiopancreatoscope. In some embodiments, the first endoscope may be the primary controller and the second endoscope may be the secondary. In this arrangement, the primary endoscope may control fluid and/or image functions of the secondary endoscope.

Advantageously, disclosed arrangements can incorporate one or more medical devices on the console reducing time between procedures for the same patient.

Advantageously, disclosed arrangements allow the addition of multiple endoscopes without additional equipment, carts, or an overall extensive setup.

Advantageously, the use of two control cartridge assemblies on the same console provide the ability to control the fluid and image controls of secondary endoscopes with the primary scope in the operator's hands, reducing the number of endoscopists necessary to complete a procedure.

The endoscope assembly may further pertain to an endoscope including a hand-piece assembly, a shaft assembly and a latch configurable between a latched configuration and an unlatched configuration. The hand-piece assembly having a housing. The shaft assembly having a housing. In the latched configuration the latch latches the housing of the hand-piece assembly to the housing of the shaft assembly. When configuring from the latched configuration to the unlatched configuration, the latch applies a separating force to separate the housing of the hand-piece assembly and the housing of the shaft assembly. The hand-piece assembly may be a reusable hand-piece assembly and/or the shaft assembly may be a single-use shaft assembly.

The latch may include a lever and the lever pivots from a first position in the latched configuration to a second position in the unlatched configuration. The latch may include a latching surface and an unlatching surface. In the latched configuration the latching surface retains the housing of the hand piece in proximity to the housing of the shaft assembly. When configuring to the unlatched configuration from the latch configuration the unlatching surface applies the separating force to separate the housings. The latch may further include a seat portion. The latching portion and unlatching portion may be located on opposing sides of the seat portion. The latch may further include a latch pivot. The latching portion and the unlatching portion may be located on opposing sides of the latch pivot. The latch may be located on the reusable hand piece. The latch may be located on the single-use shaft assembly.

Another example of an endoscope may include a hand-piece assembly and a shaft assembly. The hand-piece assembly having a housing. The shaft assembly having a housing. The endoscope may further include a biasing member and a latch The latch may include a latch configurable between a latched configuration and an unlatched configuration. The biasing member may apply a biasing force to bias the housings towards or away from one another. The latch may be configured to apply a force counter to the biasing force to force the housings away from or towards one another. For instance, the biasing member may be configured to bias the housings towards one another and the latch configured to force the housings away from one another when operated. In another instance, the biasing member may be configured to bias the housings away from one another and the latch configured to force the housings towards one another when operated and/or retain the housings together counter to the force of the biasing member. The biasing member may be a spring, a deflectable arm, a magnet, and/or a pressure chamber (e.g., pneumatic piston), just to name a few non-limiting examples.

Additionally disclosed is a latching mechanism for an endoscope having a reusable hand-piece assembly and a single-use shaft assembly including a latch and a latch receiving portion. The latch having a latching portion, an unlatching portion, and a latch handle. The latching mechanism is movable between a latched configuration and an unlatched configuration. The latch handle is movable between a first position and a second position. In the latched configuration the latch receiving portion contacts the latching portion. In the unlatched configuration the latch receiving portion and the latching portion are not in contact. When configuring from the latched configuration to the unlatched configuration, the unlatching portion may contact the receiving portion and apply a force separating the assemblies.

Additionally disclosed is a packaging system having at least a disposal bag and a container. The container may include a first portion including a recess for retaining a medical product, wherein the recess is configured to retain the medical product in a sterile environment. The container may further include a second portion configured to support the disposal bag. The second portion may be further configured to support a second bag. The container may further include a medical product box. The disposal bag and the container are both removably positioned within the medical product box prior to use of the medical product. The disposal bag may be configured to retain said medical product after said medical product has been used.

The container may further include a tray. The recess configured to retain the medical product may be defined within the tray. The tray may be fabricated from thermoformed plastic.

The container may further include a cover, and the cover may be configured and/or attachable to the tray to sterilely seal said recess. The container may be made from a recyclable material. The cover may be made from Tyvek®.

The packaging system may further include a label adhered to the cover. The label may include information pertaining to the medical product retained within the recess.

The disposal bag may be attached to the second portion of the container when the disposal bag and the container are positioned within the medical product box. The disposal bag may be detachable from the second portion of the container. The disposal bag may be adhered to the second portion. The second portion may be an exterior surface of the container. The second portion can be an interior surface of said container.

The second bag may be attached to the second portion of the container when the second bag and the container are positioned within the medical product box. The second bag may be detachable from the second portion of the container. The second bag may be adhered to the second portion.

After the medical product in its entirety or the single-use shaft assembly has been used and properly placed within the biohazard disposal bag, the biohazard disposal bag may be detached from the second portion of the packaging system and disposed of in the proper method for biohazardous waste.

After the reusable hand-piece has been placed in the second bag. The second bag may be detached from the second portion of the packaging system. The second bag with the reusable hand-piece inside may be safely transferred for reprocessing. In some examples, the medical professional or someone associated with the medical professional may ship the second bag including the reusable hand-piece back to the manufacturer for reprocessing/cleaning. Once the reprocessing operation is complete, the reusable hand-piece may be shipped back to the medical professional and/or the same practice. In other examples, the reusable hand-piece can be resold to a third party, depending on the agreement between the manufacturer and the original medical professional.

The packaging system may further include a medical product box. The disposal bag and the container are both removably positioned within the medical product box prior to use of the medical product. The second bag and the container are both removably positioned within the medical product box prior to use of the medical product.

The packaging system may further include a shipping box. Multiple medical product boxes may be packed within the shipping box.

The disposal bag may include a perimeter opening. An adhesive for adhering the disposal bag to the second portion of the container may be spaced along at least 20% of the perimeter opening.

The second bag may include a perimeter opening. An adhesive for adhering the second bag to the second portion of the container may be spaced along at least 20% of the perimeter opening.

The medical product may be an endoscope.

In other arrangements disclosed herein, the articulation controls and electronics module that incorporates control switches and navigation switches are mounted to the housing of the reusable hand-piece, and the articulation wire actuating assembly and connector of the single-use shaft assembly are mounted to the housing of the single-use shaft assembly. Accordingly, separation of the housing of the reusable hand-piece from the housing of the single-use shaft assembly separates the articulation controls from the articulation wire actuating assembly and the electronic control switches and navigation switches from the connector of the single-use shaft assembly.

The preferred embodiment may include an electronics module that includes a plurality of switches mounted to the housing of the reusable hand-piece that is in electrical communication with the wire harness associated with the single-use shaft assembly via an electrical connector. The plurality of switches mounted to the housing of the reusable hand-piece that is in electrical communication with the wire harness associated with the single-use shaft assembly via an electrical connector. The electronics module of this embodiment may further incorporate a microprocessor to minimize the number of connectors required to communicate the specific switch or combination of switches that are depressed by the user.

In another embodiment of the endoscope, the reusable hand-piece incorporates an electronics module, switches, and electrical connector while the single-use shaft assembly incorporates an optical sensor, optical sensor module, and electrical connector; wherein, a set of conductors transmits both optical and control data back to a console from the assembled endoscope.

Advantageously, the incorporation of an electronics module and switches facilitates transmission of control data while simplifying the components associated with the separable single-use shaft.

The reusable hand-piece can include a circuit board (electronics module) arranged to control at least a portion of support equipment (e.g., one or more pumps and/or valves in support equipment). One or more switches of the re-usable hand-piece can be associated with the electronics module to control the support equipment.

Advantageously, such an arrangement can eliminate the need for valves for the irrigation and aspiration lumens to be physically associated with the reusable hand-piece and/or the separable distal shaft assembly.

The switches associated with the reusable hand-piece circuit board (electronics module) can include two electrical switches actuated by discreet buttons for programmable functions at the top of the reusable hand-piece. At least one of the switches (e.g., the 2^{nd} switch from the bottom) may activate a remote suction valve. At least one of the switches (e.g., a first switch associated with the bottom button) may initiate an insufflation function via remote valve operation. At least one of the switches (e.g., a second switch associated with the bottom button) initiates a camera flush function via remote valve operation. One or more of the switches can be actuated when an operator places a finger on an exterior molded gasket covering the button. One or more of the switches can be actuated when an operator fully depresses the button.

Advantageously, the use of remote valves associated with the single-use shaft cartridge eliminates the cleaning requirements and potential contamination associated with the mechanical valves associated with the state of the art for scopes.

Current state of the art endoscopes have a vent hole located on top of the valve button which, when covered by a user's finger, provides insufflation Advantageously, a capacitive switch can provide the same user experience associated with current state of the art endoscopes with both simply requiring a finger placed on the top of the button to initiate insufflation; however, the capacitive insufflation switch eliminates cleaning requirements and potential contamination associated with the mechanical valves associated with the state of the art scopes.

The endoscope assembly can include a coupler that holds the reusable hand-piece and the single-use shaft assembly together in the assembled configuration. The coupler can include a first portion on the reusable hand-piece and a second portion on the single-use shaft assembly, wherein the first and second portions of the coupler cooperate to hold the reusable hand-piece and the single-use shaft assembly together in the assembled configuration.

The articulation controls of the reusable hand-piece can include articulation knobs. The articulation knobs can be arranged to receive articulation input from the user (e.g., in the form of rotational movement). The articulation knobs can be in communication with pulleys/cams of the separable single-use shaft to actuate control wires of the single-use shaft assembly. The user controlled articulation knobs can include two knobs having and/or rigidly is attached to concentric drive shafts.

The concentric drive shafts each have a pulley/cam engaging portion. This portion may have a non-circular cross-sectional geometric shape to aid in transmitting torque. This geometric shape can include, but is not limited to, oval, spline, square, or star, just to name a few non-limiting examples.

Advantageously, the concentric shaft drive configuration disclosed herein permits attachment of the single-use shaft assembly to the reusable hand-piece without the need to orient the articulation knobs or the distal articulating tip to obtain a neutral reference position. Regardless of the orientation of the single-use shaft assembly (coiled or straight) at the time of attachment, the articulation knobs will freely rotate while the distal shaft (insertion tube) is manipulated prior to use by the clinician.

The single-use shaft assembly can include a one or more pulleys/cams contained within a rigid housing. For example, the single-use shaft assembly may have a proximal rigid housing having a surface that supports and positions the pulleys/cams of the single-use shaft assembly to receive the pulleys/cams engaging portions of the drive shafts.

Each pulley/cam contained within the proximal rigid housing associated with the separable single-use shaft may be connected to a pair of opposing articulation wires. Each articulation wire can be rigidly fixed to the pulley/cam and on the other end to the distal tip of the articulating section of the shaft. Rotation of the pulley/cam, such as by means of a user input torque applied to the corresponding knob, results in a tensile force applied to the articulating distal section.

A preferred embodiment utilizes two pulleys/cams, each associated with a pair of articulation wires. Each pair of articulation wires provides means for articulating the distal shaft section in a single plane. Two pairs of articulation wires provide distal shaft articulation in two different planes that are perpendicular to one another, with a longitudinal axis of the proximal shaft portion passing through the intersection of the planes.

The mating features of the concentric drive shafts associated with the reusable hand-piece and the mating features of the pulleys/cams associated with the separable, single-use shaft assembly are suitable for transmitting force in the form of torque applied to the articulation knobs to tensile force in the articulation wire(s) and corresponding movement of the distal shaft articulating section.

A portion of the drive shafts, such as the pulley/cam engaging portions, may be tapered along a length of the drive shafts. For example, the drive shaft may have a portion taper from small to large in a direction towards the articulation knob. Advantageously, such an arrangement can aid in mating the drive shafts with the pinions of the single-use shaft assembly when the reusable hand-piece is coupled to the single-use shaft assembly.

The reusable hand-piece can include a brake that applies an adjustable level of rotational resistance to the articulation control (e.g., articulation control knobs). A separate brake mechanism exists for each articulation control knob and its associated articulation wire(s) for applications where at least two plane articulation is required. Furthermore, the brake mechanism(s) have been consolidated within the envelope defined by the knobs, thereby creating space within the reusable hand-piece housing for the single-use shaft assembly. Furthermore, the brake mechanism(s) do not utilize a fixed method for applying force against the friction surfaces and/or brake material, such as rotation of a threaded shaft.

Advantageously, the reusable hand-piece of the endoscope assembly can remain entirely outside of the body of a patient during an endoscopic procedure while the single-use shaft assembly has a portion positioned within the body of the patient during the endoscopic procedure. Accordingly, the reusable hand-piece and single-use shaft assembly can be separated from one another after the procedure and the single-use hand-piece discarded (or reprocessed). As there are no fluid lumens of the reusable hand-piece that must be cleaned and sterilized, the cleaning (aka “reprocessing”) effort between procedures is dramatically reduced. Additionally, as no portion of the reusable hand-piece, which is used for multiple patients, is inserted into the patient, the risk of infectious disease transmission can be dramatically reduced.

Single-use shaft assemblies can be arranged and/or provided in a variety of configurations to support upper and lower endoscopies. For example, single-use shaft assemblies may be arranged for colonoscope, gastroscope, sigmoidoscope, and/or duodenoscope procedures, just to name a few non-limiting examples. Additionally or alternatively, single-use shaft assemblies can be provided in various specialty configurations, e.g. pediatric insertion tube diameters. Advantageously, the ability to use the same reusable hand-piece for a variety of single-use shaft assemblies and/or procedures can substantially reduce capital investments by clinicians, clinician groups, and/or medical centers by eliminating the need to stock a plurality of dedicated scopes for each type of procedure, e.g. colonoscope, gastroscope, sigmoidoscope, duodenoscope, etc.

Methods of assembling an endoscope assembly, disassembling an endoscope assembly, and/or using an endoscope assembly are envisioned. Such methods can comprise connecting a housing of a reusable hand-piece to a housing of a single-use shaft assembly, wherein said reusable hand-piece has articulation controls and an electronics module and said single-use shaft assembly has an articulation wire actuating assembly and a connector; and wherein said connecting connects the articulation controls to the articulation wire actuating assembly and the control module to the connector of the single-use shaft assembly. Additionally, or alternatively, methods can comprise separating the housing of the reusable hand-piece from the housing of the single-use shaft assembly to separate the articulation controls from the articulation wire actuating assembly and the control module from the connector of the single-use shaft assembly. The methods can include providing a portion, or all, of any endoscope assembly described herein.

Advantageously, the systems, assemblies, devices, and methods disclosed herein can increase the quantity of endoscopy procedures that can be performed by a clinician and/or facility in a day by reducing and/or eliminating the time delays associated with existing reusable scopes that must undergo extensive reprocessing procedures (i.e., cleaning) between use. By using a single-use shaft, it is no longer necessary for the clinician and/or facility to reprocess (i.e., clean) the shaft and lumens of the shaft. Now the clinician and/or facility may simply wipe down the reusable hand-piece and/or sterilize it as disclosed herein and connect a new, sterilized, single-use shaft assembly to the reusable hand-piece to prepare the endoscope assembly for another procedure.

Advantageously, the systems, assemblies, devices, and methods disclosed herein can allow a clinician to perform multiple and/or various procedures even without dedicated, reusable scopes and associated reprocessing equipment, supplies, and clean water. This can be particularly advantageous in battlefield settings or remote clinics with limited resources. In these cases, the reprocessing equipment, reprocessing supplies, trained reprocessing personnel, and reprocessing laboratory setting may not be available. Advantageously, the endoscopes disclosed herein can be prepared for a new procedure by simply using a wipe and antiseptic solution to clean the exterior surfaces of the reusable hand-piece and connecting a new single-use shaft assembly.

Advantageously, the reusable hand-piece can provide user-familiar-features using higher precision reliable components associated with the articulation knobs and the brake.

Advantageously, incorporating the articulation handles and clutch into the reusable hand-piece reduces the components of the separable single-use shaft assembly thus resulting in a lower cost single-use portion of the endoscope.

The single-use shaft assemblies disclosed herein can be intended for one-time-use. Advantageously, a single-use medical device can reduce transmission of infectious diseases.

Advantageously, the preferred embodiment illustrated incorporates a series of three or more torque transmission bosses with symmetric alignment ramps that facilitate alignment of the articulation shafts with the articulation pulleys when the single-use shaft assembly is attached to the reusable hand-piece. These symmetric alignment ramps may be generated by a helical sweeping cut or by a linear angle cut, just to name a few non-limiting examples.

Applicant has also observed that expertise associated with the assessment of particular diseases and the performance of novel therapeutic procedures has become increasingly concentrated at clinical research institutions or larger healthcare facilities. Advantageously, the endoscopes disclosed herein can facilitate the dissemination of patient and image data.

Disposable flexible endoscope shafts of the present disclosure comprise a proximal portion having an insertion tube assembly and a distal portion having an articulating section assembly.

Insertion tube assemblies disclosed herein include an outer coil. Positioned within the outer coil are a plurality of compression coils. The compression coils may be arranged to increase the column strength of the outer coil along a longitudinal direction. The compression coils can be helically wound metal wires and/or spiral cut cannulas that provide for lateral flexibility. A articulation wires can be slidably positioned within lumens defined by the compression coils.

The insertion tube assembly can include a sleeve (e.g., braided sleeve) positioned around the outer coil. The sleeve preferably provides torsional strength to the insertion tube assembly. The braided sleeve can include a metal braid and/or plastic braids such as PET.

The insertion tube assembly may comprise an outer sheath positioned around the sleeve. The outer sheath may be applied as a reflowed tube or by an extruder. The outer sheath can bond to the sleeve and/or to the outer coil through apertures of the sleeve.

The articulating section assembly may comprise an articulating section having a plurality of hinges. Each hinge provides rotation around a pivot axis. The pivot axes of hinges can extend transverse to a longitudinal axis of the articulating section. Additionally, the pivot axis of one or more of the hinges can be in a different plane than the pivot axis of one or more other hinges. For example, the pivot axes of hinges can be in alternatively located in planes perpendicular to one another when the articulating section is in a straight (e.g., unbent) configuration. Advantageously, such an arrangement can provide an articulating element capable of articulating the distal tip/camera in three dimensions.

One or more hinges of the articulating section can be living hinges. In some instances, the articulating section is a unitary articulating section form. The unitary articulating section can be formed from a single piece of material. The unitary articulating section structure can be fabricated using injection molding or additive material fabrication techniques. Alternatively, the unitary articulating section can be formed by extruding a cylinder and cutting the cylinder tube with a knife, laser, milling tool, water jet, or other material removal mechanism to form the living hinges. As will be appreciated, the bending and torque fidelity characteristics of the articulating section can be configured by configuring the angles of the cuts/recesses that define the hinges and/or the distance between adjacent hinges.

In another arrangement, the articulating section structure may comprise a plurality of discrete links that, when assembled, define a plurality of concentric tab and socket pivot joints that function as a hinge. As mentioned above, each hinge (e.g., tab and socket pivot joint) can provide for rotation around a pivot axis in a single plane. Moreover, the plurality of concentric tab and socket joints can be alternatingly located in two perpendicular planes when the central axis of all links are aligned so as to provide the articulating section with multiple degrees of freedom.

An outer sheath can be positioned around the articulating section to prevent contaminants from entering the one or more hinges and/or lumens defined by the articulating section. The articulating section can include a distal cap defining an air/water nozzle, an instrument tube outlet, a camera outlet, and/or an LED outlet.

The insertion tube assembly and articulating section can be bonded together (e.g., heat or friction welding, adhesive, etc) and/or attached together with mating features on the contacting surface (e.g., threads) or with a transition tube, as shown in an illustrated embodiment. The mid-plane of the transition tube can be located at the transition between the insertion tube assembly and the articulating section, and the transition tube can be bonded (e.g., swaged or adhered with adhesive) onto both the insertion tube assembly and the distal articulating section to form a secure attachment. The transition tube can be deformable to allow deflection of the flexible endoscope shaft at the transition.

The insertion tube assemblies disclosed herein can be manufactured using a continuous (e.g., reel-to-reel) manufacturing process. The braided sleeve can be applied around the outer coil during the continuous manufacturing process. Additionally, the outer sheath can be applied during the continuous manufacturing process. For example, the assembly of the outer coil and surrounding braided sleeve can pass through one or more extrusion heads during the continuous manufacturing process to apply the outer sheath to the portion of the insertion tube assembly. Such a process can create a smooth outer sheath that is integrally bonded to the outer coil and/or braided sleeve. The outer sheath may have a varying durometer along a length of the shaft.

After positioning of the outer sheath around the assembly, the shaft may be cut to the desired length and compression coils and/or articulation wires inserted into an interior of the outer coil.

Advantageously, providing continuous manufacturing of an insertion tube assembly can reduce the cost of manufacturing the endoscope shaft assembly and increase production speed. Accordingly, in certain aspects, the present disclosure provides a low-cost, flexible endoscope shaft and method of manufacturing same. As the insertion tube assembly can be manufactured continuously, desired lengths of insertion tube assembly, or a portion thereof, can be cut to length after the outer sheath extrusion process or cut from a finish goods reel. Advantageously, continuous techniques for fabricating the insertion tube (e.g., reel-to-reel techniques) avoid braiding and coating the insertion tube in discrete sections using labor intensive processes.

As will be appreciated, the insertion tube assemblies disclosed can provide containment of wiring, tubes, and actuation wires of the endoscope shaft while having torsional and compressive strength sufficient to advance the articulating section assembly through tortuous vessels of a patient.

Further disclosed are articulating joints that comprise an articulating section assembly, one or more breakaway struts, a proximal link containing compression coil cavities and fingers that engage the interior of an insertion tube, and a distal link with keyed features that engage a distal cap.

The articulating joint assemblies disclosed herein can comprise a central core with lumens for, but not limited to use for, instrument tubes, air tubes, water tubes, camera wire harness, LED wire harness, compression coils, steering wires, or any combination thereof. One or more of the lumens can be positioned entirely within the periphery of the central core and/or having a closed circumference.

The articulating joint assemblies can comprise a proximal link, a plurality of links, and a distal link. Through the length of the articulating joint are channels in each link for steering wires (e.g., articulation wires) to pass.

The proximal link can comprise compression coil cavities and fingers that engage the interior of an insertion tube for connection with a shaft. Additionally, the anterior end of the proximal link can comprise a means of interfacing with the posterior link in the plurality of links which allows for articulation between the links.

The articulating section assembly can comprise a series of one or more links of the same diameter having an anterior and posterior face. The posterior face interfaces with the anterior face of the preceding link. Each means of interfacing preferably allows the individual links in the articulating section assembly to articulate.

The articulating section assembly can comprise an articulating section having a plurality of hinges. Each hinge provides rotation around a pivot axis. The pivot axes of hinges can extend transverse to a longitudinal axis of the articulating section. Additionally, the pivot axis of one or more of the hinges can be in a different plane than the pivot axis of one or more other hinges. For example, the pivot axes of hinges can be in alternatively located in planes perpendicular to one another when the articulating section is in a straight (e.g., unbent) configuration. Advantageously, such an arrangement can provide an articulating element capable of articulating the distal tip/camera in three dimensions.

One or more hinges of the articulating section can be living hinges. In some instances, the articulating section is a unitary articulating section form. The unitary, articulating section can be formed from a single piece of material. The unitary articulating section structure can be fabricated using injection molding or additive material fabrication techniques. Alternatively, the unitary articulating section can be formed by extruding a cylinder and cutting the cylinder tube with a knife, laser, milling tool, water jet, or other material removal mechanism to form the living hinges. As will be appreciated, the bending and torque fidelity characteristics of the articulating section can be configured by configuring the angles of the cuts/recesses that define the hinges and/or the distance between adjacent hinges.

In another arrangement, the articulating section structure can comprise a plurality of discrete links that, when assembled, define a plurality of concentric tab and socket pivot joints that function as a hinge. As mentioned above, each hinge (e.g., tab and socket pivot join) can provide for rotation around a pivot axis in a single plane. Moreover, the plurality of concentric tab and socket joints can be alternatingly located in two or more planes (e.g., perpendicular planes) when the central axis of all links are aligned so as to provide the articulating section with multiple degrees of freedom.

The proximal link can comprise a posterior face with one or more recess for capturing the compression coils that engage the proximal link and resist the steering wire pull force, an anterior face with a means of interfacing the plurality of links, and a cylindrical shape.

The distal link can comprise an anterior face with keyed features that engage a distal cap, a posterior face with a means of interfacing the plurality of links, and a cylindrical shape. The posterior face can comprise a means of interfacing with the anterior face of the anterior link of the plurality of links and allows for articulation between the links.

The articulating join assembly can include one or more looped steering wires (e.g., a pair of looped steering wires) which negate the need for steering wire (e.g., articulation wire) termination in the distal link, where space is limited.

A single looped steering wire for single plane steering may have segments retained by features in the distal link 180 degrees apart, with the radial bend of the loop being retained by one or more feature in the distal cap thus maintaining an open lumen for passage of internal tubes and control wires. The looped steering wire may be secured to the distal link utilizing either adhesive and/or mechanical means.

Two looped steering wires for two plane steering may have each looped wire retained by features either 90 or 180 degrees apart, with the radial bend of each steering wire being retained by one or more features that maintain an open lumen for passage of internal tubes and control wires. The looped steering wires may be secured to the distal link utilizing either adhesive and/or mechanical means. When fully assembled, the pair of looped steering wires may provide four discrete wire terminations at the proximal end to apply a steering pull force on the distal link.

The plurality of links may be molded or fabricated utilizing an additive manufacturing process. Manufacturing processes may facilitate the addition of support struts that maintain spacing between and alignment of the links to facilitate insertion of steering wires into steering wire lumens and tubing and/or wiring within the central lumen. The struts may ensure the correct number and types of links are used when assembling the articulating section. When assembled, the struts may be broken away from the links at attachment points, thus allowing the plurality of links to articulate.

One or more breakaway struts may be positioned laterally along the length of the plurality of links, attaching to each link individually, facilitating the ease of alignment of control cables, tools, and internal tubes through the lumens of the articulating joint for use with the medical device.

One or more breakaway struts may be positioned laterally along the length of the proximal link, the plurality of links, and the distal links, attaching to each link by one or more connection points, facilitating the ease of alignment of control cables, tools, and internal tubes through the lumens of the articulating joint.

Any of the inventive aspects and embodiments discussed herein may be used independently or in combination with each other. Other aspects, objectives, and advantages of the present disclosure will become more apparent from the following detailed description when taken in conjunction with the accompanying drawings. Further forms, objects, features, aspects, benefits, advantages, and embodiments of the present invention will become apparent from a detailed description and drawings provided herewith.

### BRIEF DESCRIPTION OF THE DRAWINGS

FIGS. 1, 2, 3, 4, 5 and 6 illustrate an endoscope assembly.

FIGS. 7, 8 and 9 illustrate exploded views of the endoscope assembly of FIG. 1.

FIG. 10 illustrates a cross-sectional view of the articulation control and braking portion of the endoscope assembly of FIG. 1.

FIGS. 11, 12, 13 and 14 illustrate a reusable hand-piece of an endoscope assembly.

FIGS. 15 and 16 illustrate a disposable shaft assembly of an endoscope assembly.

FIG. 17A illustrates a perspective view of a pulley assembly.

FIG. 17B illustrates an exploded view of the pulley assembly of FIG. 17A.

FIG. 17C illustrates an exploded schematic view of a pulley assembly and associated articulation shafts and a brake shaft.

FIGS. 17D, 17E, 17F, and 17G illustrate exploded schematic views of a rack and pinion assembly.

FIG. 18 illustrates a cross-sectional view of the articulation knob and brake assembly.

FIG. 19 illustrates a perspective view of the articulation knob and brake assembly.

FIG. 20 illustrates a side view of the articulation knob and brake assembly.

FIG. 21 illustrates an exploded view of the articulation knob and brake assembly.

FIG. 22 illustrates a cross-sectional view of the up-down articulation knob and brake portion of the articulation knob and brake assembly.

FIG. 23 illustrates a perspective exploded view of the up-down articulation brake lever and up-down articulation brake outer housing.

FIG. 24 illustrates a perspective view of the up-down articulation brake lever and up-down articulation brake outer housing.

FIG. 25 illustrates an exploded view of up-down articulation hub, the up-down articulation brake detent and the up-down articulation brake caliper.

FIG. 26 illustrates a perspective view of the up-down articulation brake outer housing and the up-down articulation shaft.

FIG. 27 illustrates a perspective view of the up-down articulation brake outer housing of FIG. 26.

FIG. 28 illustrates a side-elevational view of the articulation knob and brake assembly without the articulation knobs, outer housings, the up-down articulation brake lever and without the articulation brake inner housings.

FIG. 29 illustrates a cross-sectional view of the up-down articulation brake knob and brake assembly in a configuration wherein the brake is disengaged from the up-down articulation brake outer housing.

FIG. 30 illustrates a perspective view of the left-right articulation brake outer housing and the left-right articulation shaft.

FIG. 31 illustrates a perspective view of the left-right articulation brake shaft and the left-right articulation brake hub.

FIG. 32 illustrates a perspective view of the left-right articulation brake shaft and left-right articulation brake hub of FIG. 31.

FIG. 33 illustrates a schematic diagram of electrical circuits of the endoscope assembly.

FIG. 34 illustrates a schematic diagram of another embodiment of electrical circuits of the endoscope assembly.

FIG. 35 illustrates a schematic diagram of another embodiment of electrical circuits of the endoscope assembly.

FIG. 36 illustrates an exploded view of the single-use shaft assembly and fluid paths thereof.

FIG. 37 illustrates a method and device of sterilizing the reusable hand-piece using an autoclave.

FIG. 38 illustrates a method and device of sterilizing the reusable hand-piece using ultraviolet radiation via quartz lamps.

FIG. 39 illustrates a method and device of sterilizing the reusable hand-piece using ultraviolet radiation via light emitting diodes (LEDs).

FIG. 40A illustrates a perspective view of an endoscope, a cartridge and a console including a control valve assembly.

FIG. 40B illustrates a perspective view of another example of an endoscope, a cartridge and a console including a control valve assembly.

FIG. 41A illustrates a perspective view of the console of FIG. 40A.

FIG. 41B illustrates a perspective view of the console of FIG. 40B.

FIG. 42A illustrates a perspective view of the control valve assembly of FIG. 40A.

FIG. 42B illustrates a perspective view of the control valve assembly of FIG. 40B.

FIG. 43A illustrates a front view of the control valve assembly of FIG. 40A.

FIG. 43B illustrates a front view of the control valve assembly of FIG. 40B.

FIG. 44A illustrates a right-side view of the control valve assembly of FIG. 40A.

FIG. 44B illustrates a right-side view of the control valve assembly of FIG. 40B.

FIG. 45A illustrates a top view of the control valve assembly of FIG. 40A.

FIG. 45B illustrates a top view of the control valve assembly of FIG. 40B.

FIG. 46A illustrates a back view of the cartridge of FIG. 40A.

FIG. 46B illustrates a back view of the cartridge of FIG. 40B.

FIG. 46C illustrates a back view of the cartridge of FIG. 40B with the cover removed.

FIG. 47A illustrates a front-right perspective view of the cartridge of FIG. 40A.

FIG. 47B illustrates a front-right perspective view of the cartridge of FIG. 40B.

FIG. 48A illustrates a back-left perspective view of the cartridge of FIG. 40A.

FIG. 48B illustrates a back-left perspective view of the cartridge of FIG. 40B.

FIG. 49A illustrates an exploded perspective view of the cartridge and control valve assembly of FIG. 40A.

FIG. 49B illustrates an exploded perspective view of the cartridge and control valve assembly of FIG. 40B.

FIG. 50A illustrates a perspective view of the cartridge securely inserted into the control valve assembly of FIG. 40A.

FIG. 50B illustrates a perspective view of the cartridge securely inserted into the control valve assembly of FIG. 40B.

FIG. 51 illustrates a schematic diagram of the fluid portion of an endoscopic system.

FIG. 52 illustrates a schematic diagram of an electrical portion of an endoscopic system.

FIG. 53 illustrates a schematic view of a sealed medical package containing a sterilized cartridge and a sterilized single-use shaft assembly.

FIG. 54A illustrates a perspective view of another embodiment of two endoscopes, two cartridges and a console including two control valve assemblies.

FIG. 54B illustrates a perspective view of another example of FIG. 54A.

FIG. 55 illustrates a perspective view of an endoscope assembly having a latching mechanism in a latched configuration.

FIG. 56 illustrates a perspective view of the endoscope from FIG. 55 in an unlatched configuration.

FIG. 57 illustrates a perspective view of the endoscope from FIG. 55 in an unlatched and separated configuration.

FIG. 58 illustrates an enlarged perspective view of the endoscope from FIG. 57.

FIG. 59A illustrates a perspective view of a packaging system for a medical product.

FIG. 59B illustrates a perspective view of a packaging system of FIG. 59A with two bags.

FIG. 60 illustrates the packaging system of FIG. 59A with a portion of a cover peeled away from the top of a tray to show a recess defined within the tray.

FIG. 61 illustrates a cross sectional view of the tray of the packaging system of FIG. 59A on a cart.

FIG. 62 illustrates a disposal bag of the packaging system of FIG. 59A.

FIG. 63 illustrates the packaging system of FIG. 59A packaged within a medical product box.

FIG. 64 illustrates medical product boxes as shown in FIG. 63 packaged within a shipping box.

FIG. 65 illustrates the packaging system of FIG. 59A with the cover and medical product removed.

FIG. 66 illustrates the disposal bag of FIG. 62 with a used medical product inserted within the disposal bag.

FIG. 67 illustrates a tray of the packaging system of FIG. 59A being recycled after use.

FIG. 68 illustrates an isometric view of a reusable endoscope with mechanical valves for suction, insufflation, and camera flush controls.

FIG. 69 illustrates an isometric view of a separable reusable hand-piece with buttons that actuate electrical switches including an integral capacitive touch switch.

FIG. 70 illustrates the function of the combined insufflation/camera flush button that incorporates a capacitive switch.

FIG. 71 illustrates an exploded isometric view of a separable reusable hand-piece illustrating the locations of the buttons, molded gasket, switches, control board, and capacitive switch antennae.

FIGS. 72, 73 and 74 illustrate an endoscope assembly.

FIG. 75 illustrates a rear view of a single-use shaft assembly separated from a reusable hand-piece.

FIG. 76 illustrates a front view of the single-use shaft assembly separated from the reusable hand-piece.

FIG. 77 illustrates a cross-section front view of the single-use shaft assembly separated from the reusable hand-piece.

FIG. 78 illustrates a cross-section front isometric view of the single-use shaft assembly separated from the reusable hand-piece.

FIG. 79 illustrates a cross-section rear isometric view of the articulation knob and brake assembly separated from the pulley assembly.

FIG. 80 illustrates a cross-section rear isometric view of the shaft assembly separated from the pulley and socket torque transmission assembly.

FIG. 81 illustrates an exploded view of the shaft assembly separated from the pulley and socket torque transmission assembly.

FIG. 82 illustrates the left-right brake shaft separated from the socket torque transmission boss.

FIG. 83 illustrates the up-down articulation shaft separated from the up-down articulation pulley.

FIG. 84 illustrates the pulley and socket torque transmission assembly with steering wires shown

FIG. 85 illustrates a perspective view of a disposable flexible endoscope shaft assembly.

FIG. 86A illustrates a perspective view of an insertion tube assembly.

FIG. 86B illustrates a perspective view of a distal end of the insertion tube assembly.

FIG. 87 illustrates an exploded view of the insertion tube assembly.

FIG. 88 illustrates a perspective view of the articulating section assembly.

FIG. 89A illustrates an exploded view of the articulating section assembly.

FIG. 89B is a partial exploded view of the articulating section assembly.

FIG. 90A illustrates a perspective view of the unitary articulating section in a straight configuration.

FIG. 90B illustrates a perspective view of the unitary articulating section in a deflected configuration.

FIG. 90C illustrates a close-up view of the unitary articulating section.

FIG. 91A illustrates a close-up of the proximal end of the unitary articulating section of FIGS. 90A-90C.

FIG. 91B illustrates a close-up of the distal end of the unitary articulating section of FIGS. 90A-91A.

FIG. 92 illustrates a perspective view of an articulating link assembly.

FIG. 93A illustrates a perspective view of an articulating link.

FIG. 93B illustrates a perspective view of the proximal articulating link.

FIG. 93C illustrates a perspective view of the distal articulating link.

FIG. 94 illustrates a perspective view of the articulation pull wire and termination ring assembly.

FIG. 95 is a flowchart illustrating a process for manufacturing disposable flexible endoscope shafts disclosed herein.

FIG. 96 illustrates a first portion of a manufacturing arrangement.

FIG. 97 illustrates a second portion of a manufacturing arrangement.

FIGS. 98A-98D illustrate an articulating joint assembly with one or more breakaway struts connected to an articulating section assembly with one or more connection points on each individual articulating link.

FIGS. 98E-98H illustrate an articulating joint assembly with one or more breakaway struts connected to a proximal link, an articulating section assembly, and a distal link with one or more connection points one each individual articulating link.

FIG. 99 illustrates an exploded view of an articulating joint, distal cap, and tube assembly.

FIG. 100A-100C illustrate a proximal link.

FIG. 101A-101C illustrate a distal link.

FIG. 102A-102B illustrate a distal link and articulation steering wire assembly.

FIG. 103A-103C illustrate a distal cap.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. It will be apparent to those skilled in the relevant art that some features may not be shown for the sake of clarity.

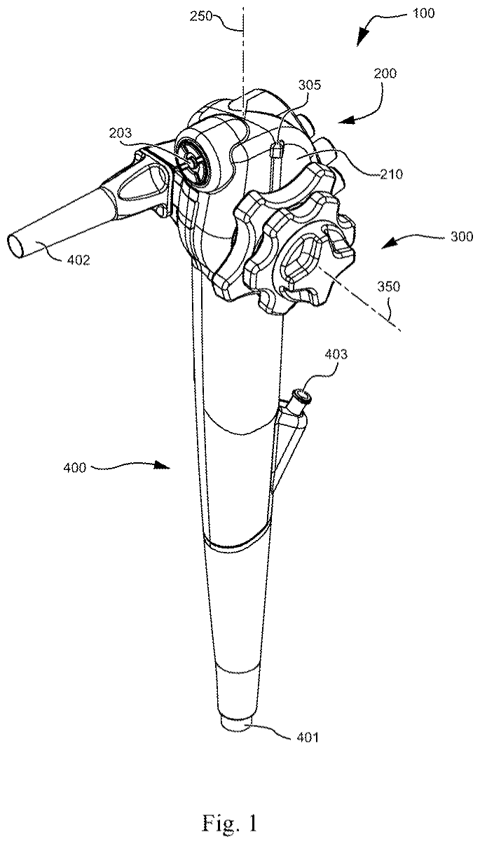

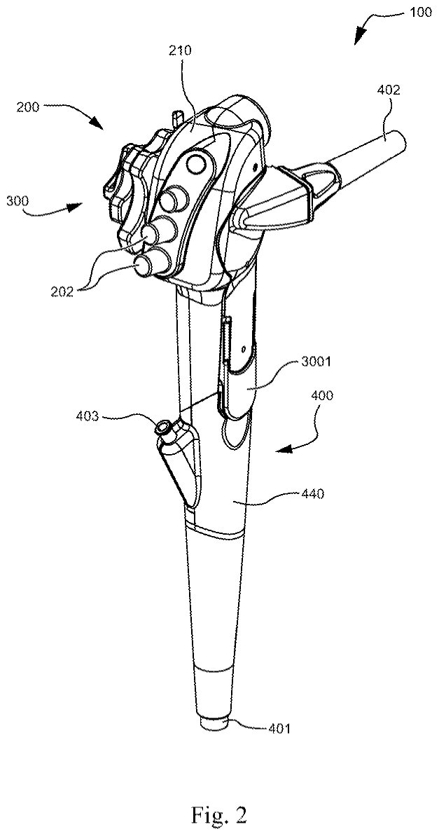

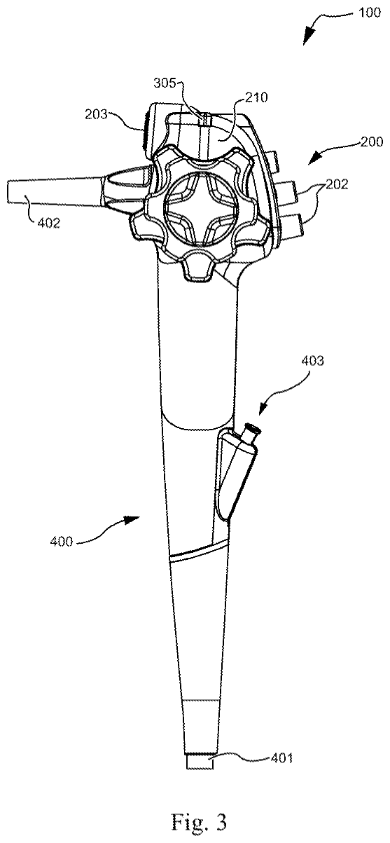

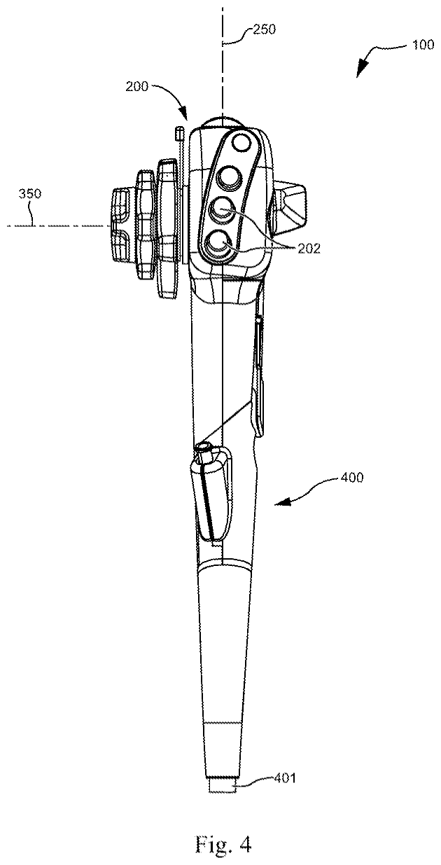

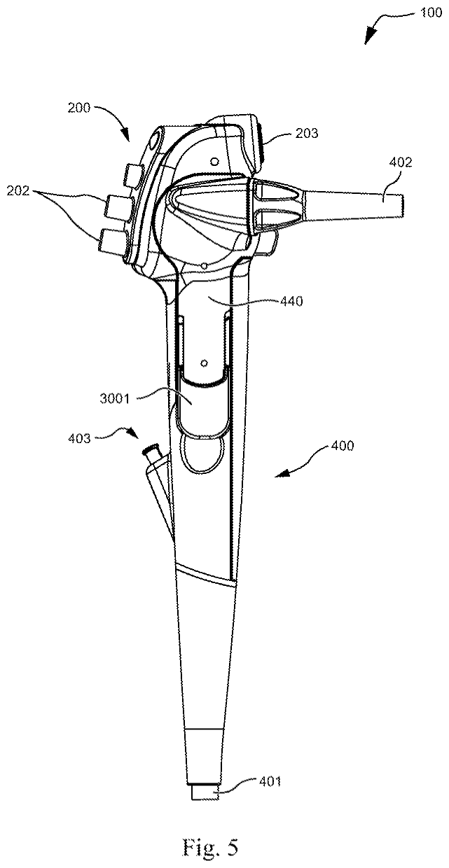

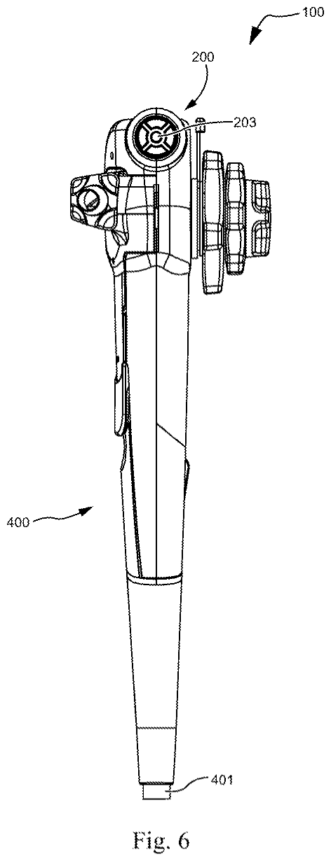

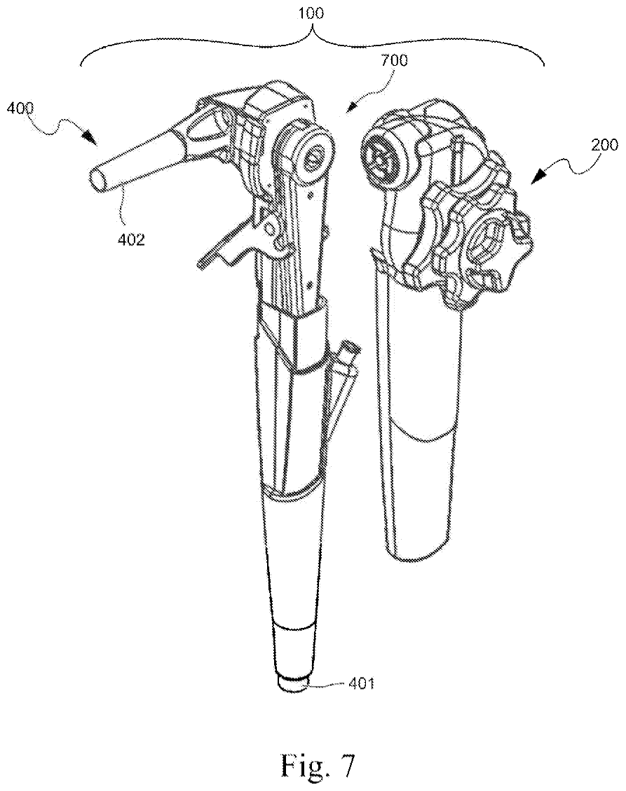

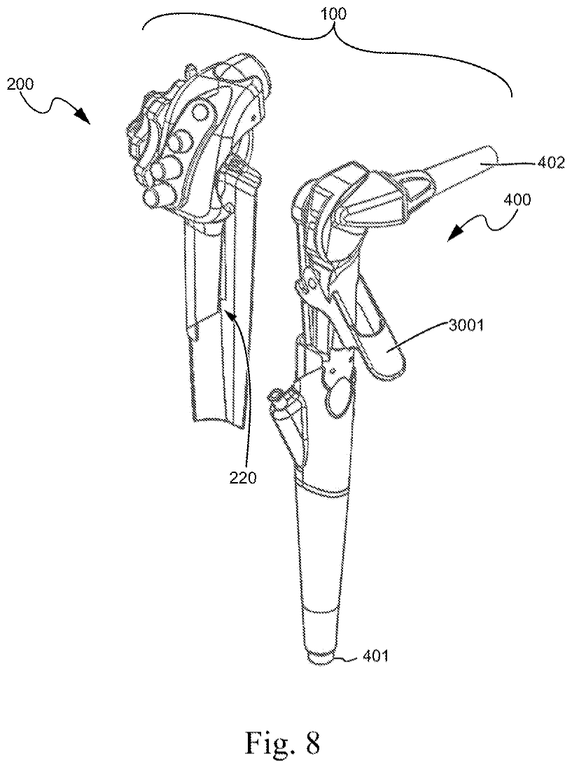

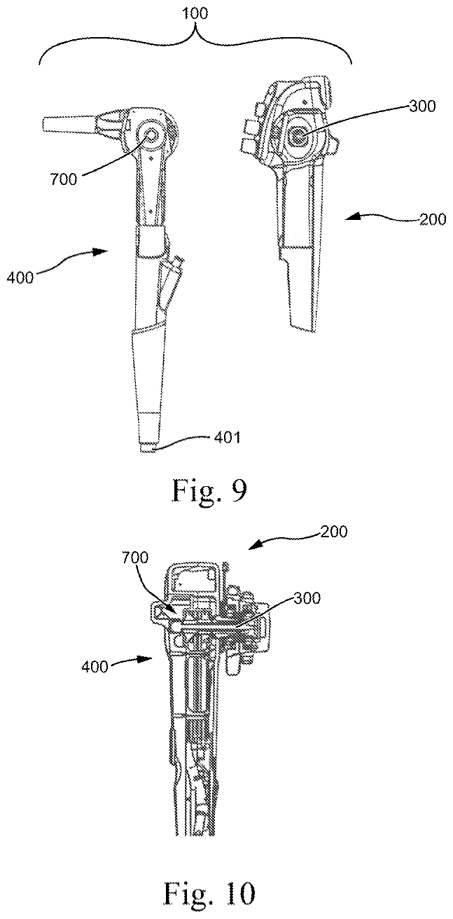

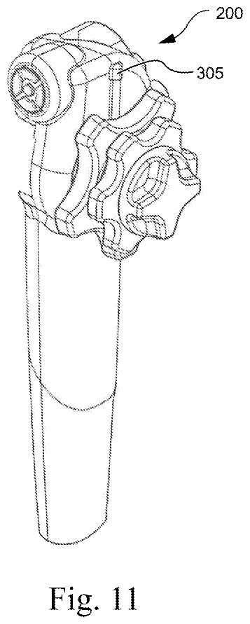

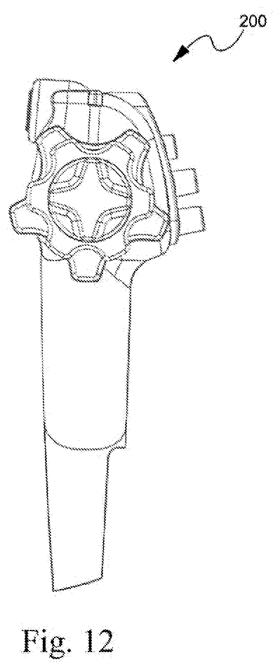

The present disclosure provides an endoscope assembly comprising a reusable hand-piece and a single-use shaft assembly. FIGS. 1 through 10 illustrate an endoscope assembly 100. More particularly, FIGS. 1 through 6 illustrate the endoscope assembly 100 with the reusable hand-piece 200 attached to the single-use shaft assembly 400. FIGS. 7-9 illustrate the endoscope assembly 100 with the reusable hand-piece 200 detached from the single-use shaft assembly 400.

The reusable hand-piece 200 is selectively attachable and detachable from the single-use shaft assembly 400 such that the reusable hand-piece may be used serially with a number of single-use shaft assemblies while the single-use shaft assembly can be discarded after a single use. The reusable hand-piece and/or single-use shaft assemblies may include a lock for selectively locking the reusable hand-piece and single-use shaft assembly to one another. A latch handle 3001 (e.g., a lever) for actuating the latching mechanism connecting the reusable hand-piece to the single-use shaft assembly may be positioned on the reusable hand-piece and/or the single-use shaft assembly.

The reusable hand-piece can include a hand-piece housing 210 supporting an articulation knob and brake assembly 300; control switches 202 for operating light(s), such as light emitting diodes, and/or flush and/or aspiration fluid paths; one or more navigation switches 203 for controlling a console communicating with the endoscope assembly; and/or an electrical connector 204 for electrically coupling the reusable hand-piece to the single-use shaft assembly.

The single-use shaft assembly can include a insertion tube 401 for insertion within the body of a patient, a tubing and electrical connector 402 connected to a console, an access port 403 for selectively accessing a lumen of the single-use shaft assembly (e.g., for taking a sample such as a biopsy) and/or for delivering a fluid through a lumen of the single-use shaft assembly, and/or an electrical connector 404 for electrically coupling the single-use shaft assembly to the reusable hand-piece.

A tubing and/or electrical connector 402, sometimes referred to as an “umbilical”, extends from the endoscope assembly to a console suitable for use with the endoscope assembly. The tubing and/or electrical connector can extend from the single-use shaft assembly and/or from the reusable hand-piece.

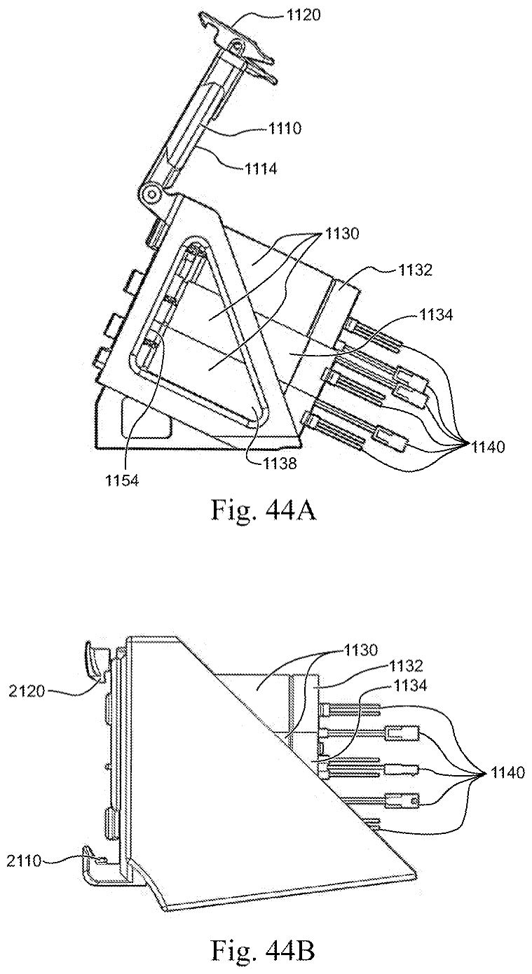

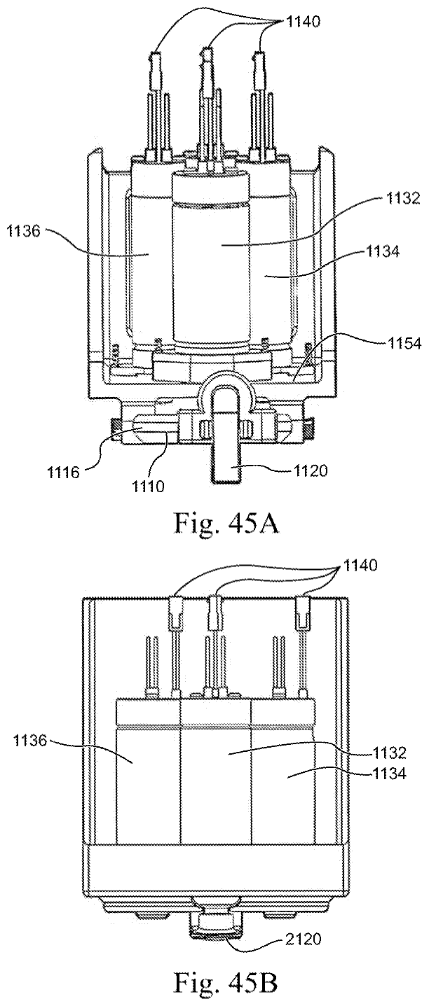

A console (shown, for example, in FIGS. 44-45) may provide electrical power, fluids and/or fluid pressure, vacuum pressure, and/or send and/or receive electrical signals with the endoscope assembly. The console may include a monitor for visualizing signals received from the endoscope assembly such as a video feed from a camera at the distal end of the single-use shaft assembly.

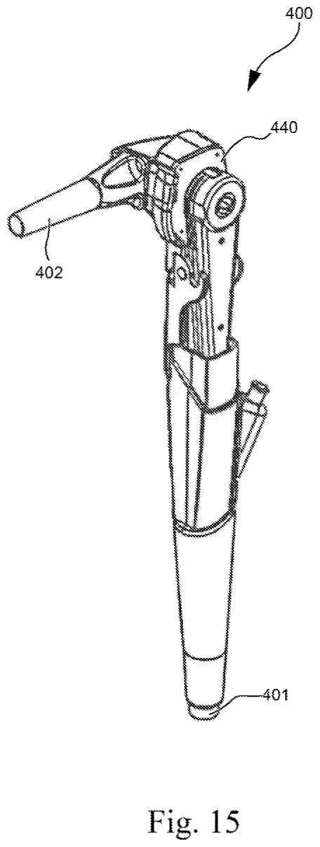

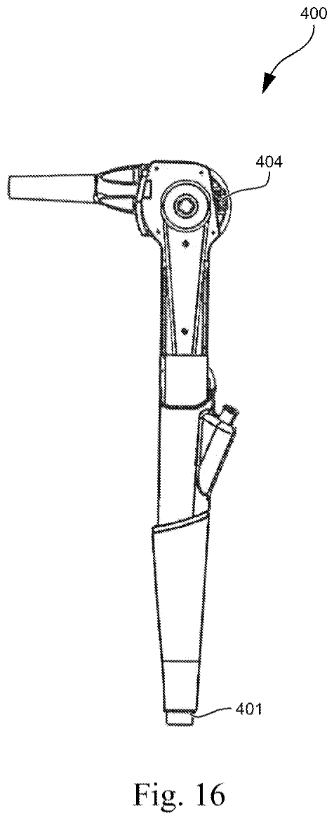

The single-use shaft assembly can include one or more pulley assemblies and/or rack and pinion assemblies, such as those shown in FIGS. 15-17G. Pulley assemblies can include at least one pulley 450 arranged to receive an end of an articulation shaft. Preferably, rotation of the articulation knob associated with the articulation shaft rotates the at least one pulley. Preferably, the at least one pulley and end of the articulation shaft have a geometry (e.g., a mating feature) for rotationally coupling the pulley and articulation shaft to one another. Preferably the mating feature does not rely solely on friction between the articulation shaft and pulley/pinion. Preferably the mating features include a geometric interference between the articulation shaft and pulley.

Associated with the at least one pulley is an articulation wire or a pair of articulation wires 507 (e.g., two separate wires or one continuous wire looped over the pulley and having wire segments extending from opposing sides of the pulley) configured to bend a distal shaft section of the single-use shaft assembly upon actuation. The articulation wires may be fixed (e.g., tied or fixed by anchors 456) to the pulley and/or extend around at least a portion of the pulley (e.g., loop around the pulley). Preferably, the one or more pulley assemblies include two pulleys (one for each articulation shaft) and articulation wires (at least one for each pulley). Preferably the articulation wires are configured to bend the distal shaft section in orthogonal planes.

The one or more pulleys and associated one or more articulation wires may be contained within a pulley housing comprising a first housing portion 460 and a second housing portion 464. Such housing portions may cooperate to enclose the pulley and associated articulation wire(s). Preferably the housing portions resist the articulation wire(s) from separating from the pulley(s). The pulley housing can defines openings 470 for receiving articulation shafts and/or a brake shaft of the articulation control assembly.

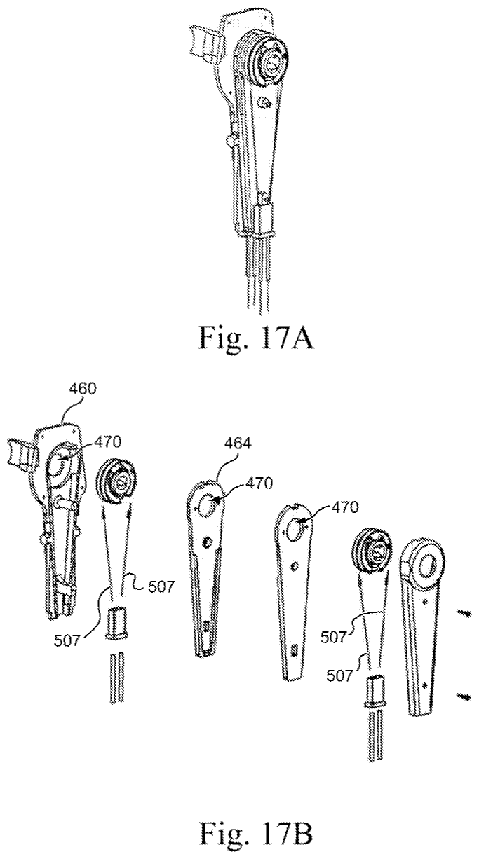

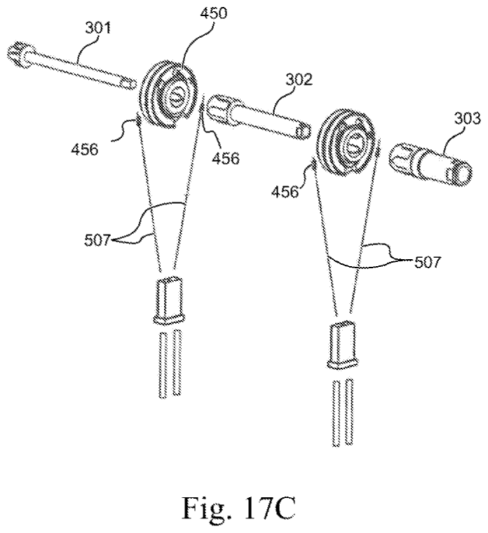

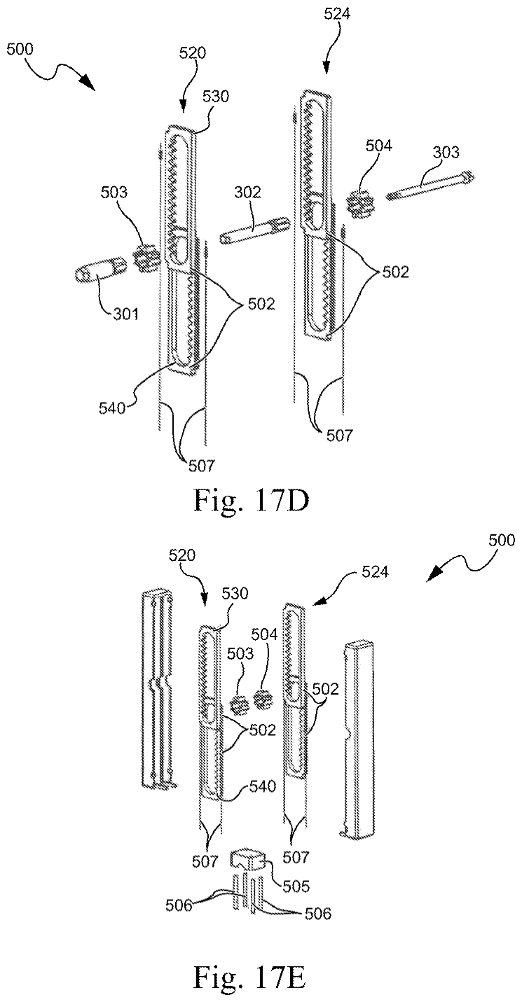

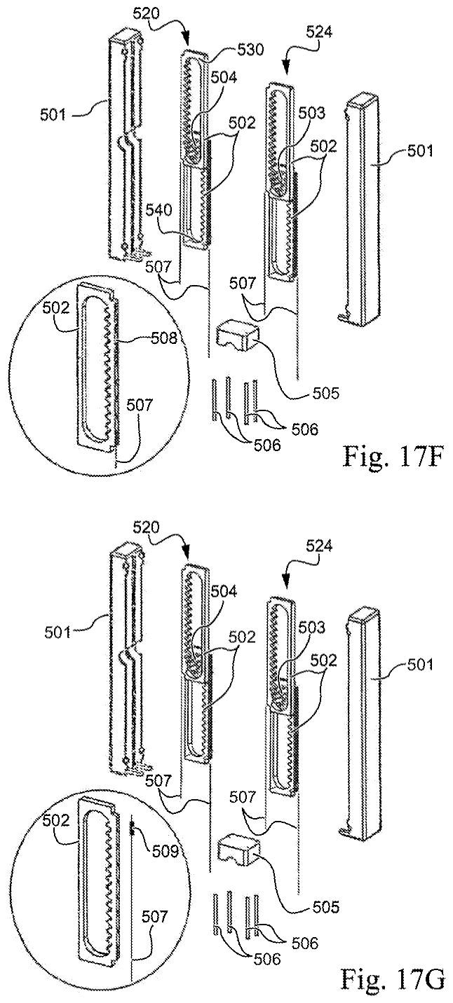

Turning to FIGS. 17D-G, the single-use shaft assembly may include a rack assembly 500 for actuating a distal, articulating end of the single-use shaft assembly. The rack assembly can comprise racks 502 forming a left-right articulation rack 520 and an up-down articulation rack 524. One or more of the articulation racks can include an articulation pinion 503, 504 and a first articulation rack portion 530, and/or a second articulation rack portion 540, associated with the articulation pinion. Upon rotation of the articulation pinion, one or more articulation rack portions articulate to actuate one or more articulation wires 507 to extending through a length of the shaft of the single-use shaft assembly. For example, rotation of an up-down articulation pinion may translate a first articulation rack portion in a first direction (e.g., upwards) and a second articulation rack portion in a second direction (e.g., downwards) so as to deflect a distal end region of the single-use shaft assembly either up or down.

In addition to the articulation racks and articulation pinions described above, the rack assembly may include an articulation coil termination block 505 that receives one or more articulation coils 506 wrapping around respective articulation pull wires 507 connected to the articulation racks 502. The articulation pull wires can be attached to the articulation racks by articulation pull wire adhesive 508 and/or an articulation pull wire crimp tube 509. The rack assembly/assemblies may include a housing 501 arranged to retain the racks and pinion and limit the racks to translational movement.

When the reusable hand-piece is attached to the single-use shaft assembly, the articulation knob and brake assembly of the reusable hand-piece engages the rack assembly of the single-use shaft assembly such that rotation of one or more knobs of the articulation knob and brake assembly actuates one or more components to the rack assembly. For example, the articulation knob and brake assembly may engage pinions of the rack assembly such that rotation of one or more knobs of the articulation knob and brake assembly rotates one or more articulation pinions of the rack assembly to actuate one or more articulation racks and articulation wires of the single-use shaft assembly.

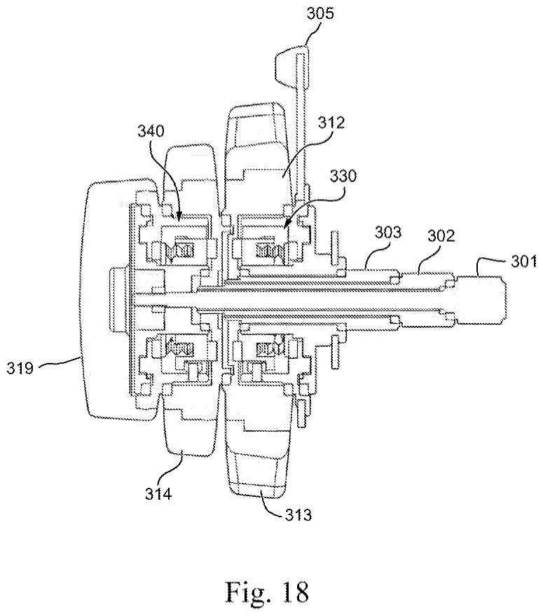

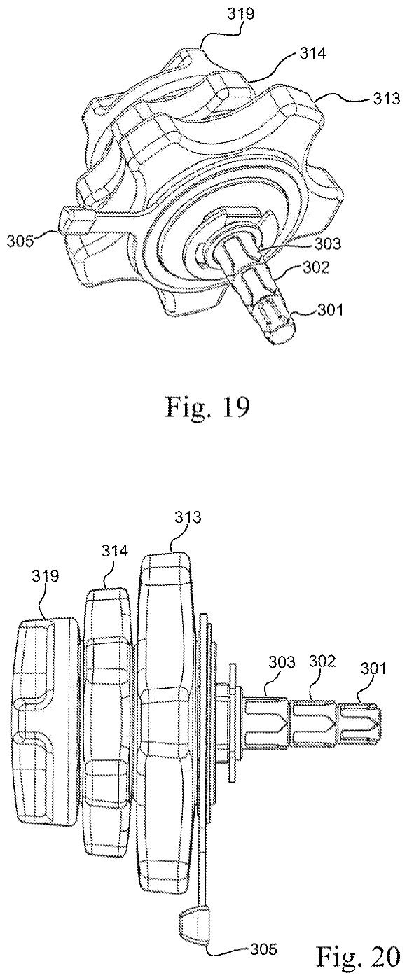

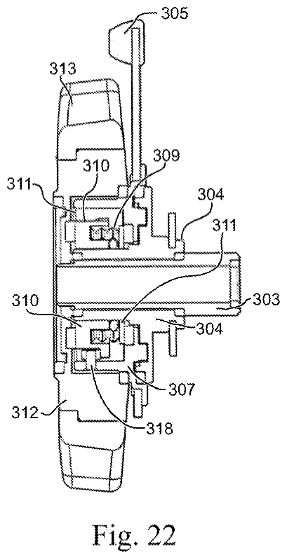

With exemplary reference now to FIGS. 18-32, the articulation knob and brake assembly of the reusable hand-piece can include an up-down articulation knob 313, a left-right articulation knob 314, an up-down articulation brake 330 and a left-right articulation brake 340. During operation, when the reusable hand-piece is attached to the single-use shaft assembly, articulation of the up-down articulation knob articulates a distal portion of the single-use shaft assembly in a first plane and actuation of the left-right articulation brake knob actuates the distal portion of the single-use shaft assembly in a second plane transverse to the first plane. Preferably, the second plane is orthogonal to the first plane.

When the reusable hand-piece is coupled to the single-use shaft assembly, actuation of the up-down articulation brake lever 305 engages the up-down articulation brake 330 that resists rotation of the up-down articulation knob relative to a portion of the endoscope assembly, such as the housing 210 of the reusable hand-piece. Actuation of the left-right articulation brake knob 319 engages the left-right articulation brake 340 that resists rotation of the left-right articulation knob relative to a portion of endoscope assembly, such as a housing 440 of the single-use shaft assembly.

The reusable hand-piece and the single-use shaft assembly may be configured to mate with one another. For example, the reusable hand-piece assembly may define a recess 220 arranged to receive a portion of the single-use shaft assembly, or vice versa. In many embodiments, the recess of the reusable hand-piece assembly arranged to receive a portion of the single-use shaft assembly will be on a side of the reusable hand-piece assembly opposing the side having the knobs of the articulation knob and brake assembly. One of the reusable hand-piece assembly or the single-use shaft assembly may surround at least a portion of the other, partially and/or completely, on two or more opposing sides.

A portion of the single-use shaft assembly may be receivable in the reusable hand-piece, or vice versa, along a direction parallel-to or transverse to a longitudinal axis of the reusable hand-piece or single-use shaft assembly. For example, the reusable hand-piece may receive the single-use shaft assembly along a direction orthogonal to a longitudinal axis 250 of the reusable hand-piece. In many instances, when joining the reusable hand-piece and single-use shaft assembly to one another, the single-use shaft assembly will move, relative to the reusable hand-piece, along a direction parallel to a rotation axis 350 of the articulation knob and brake assembly.

When the reusable hand-piece assembly is attached to the single-use shaft assembly, the articulation knob and brake assembly cooperates with the single-use shaft assembly to articulate a distal segment of the single-use shaft assembly and/or resist rotation of one or more articulation knobs relative to the endoscope assembly. For example, as discussed in more detail elsewhere herein, one or more articulation shafts 302, 303 and/or brake shafts 301 of the articulation knob and brake assembly may engage the rack assembly and/or housing of the single-use shaft assembly. As illustrated in FIG. 10, one or more shafts of the articulation knob and brake assembly extend through the rack assembly and engage the housing of the single-use shaft assembly on an opposing side of the rack assembly from the articulation knob and brake assembly.

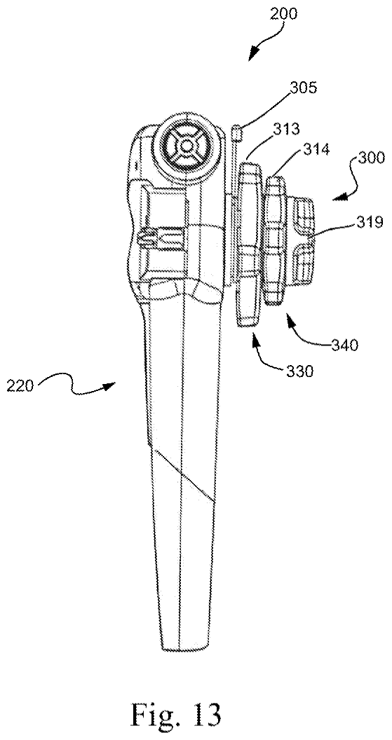

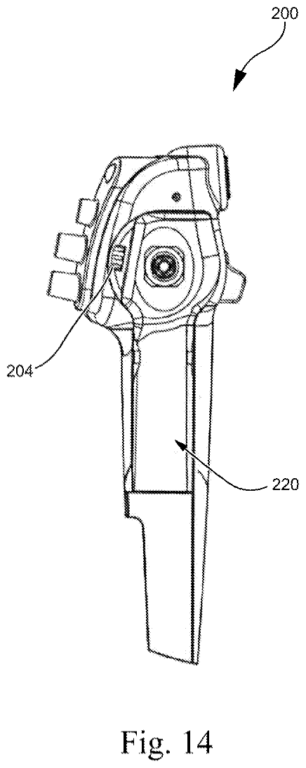

As shown in FIGS. 13 and 14, in many embodiments a portion of the articulation knob and brake assembly extends into the recess of the reusable hand-piece arranged to receive a portion of the single-use shaft assembly.

Turning back now to FIGS. 18-32, the articulation knob and brake assembly is shown in greater detail. Regarding the up-down articulation control by the articulation knob and brake assembly, the up-down articulation knob 313 is supported on an up-down articulation brake outer housing 312. Positioned within the up-down articulation brake outer housing 312 is an articulation brake inner housing 307, an articulation brake position detent 308, an articulation brake spring 309, an up-down articulation brake caliper 310, and an articulation brake friction disc 311.

Connected to the articulation brake inner housing 307 is the up-down articulation brake lever 305. Extending through the up-down articulation brake lever 305 is an up-down articulation hub 304 and an up-down articulation shaft 303. The up-down articulation hub 304, which is positioned within the articulation brake inner housing 307, supports the articulation brake position detent 308 and the up-down articulation brake caliper 310. Rotation of the up-down articulation hub 304 is fixed relative to the housing 210 of the reusable hand-piece 200 which, in turn, fixes rotation of the articulation brake position detent 308 and the up-down articulation brake caliper 310 relative to the housing of the reusable hand-piece as well.

Upon rotation of the up-down articulation brake lever 305 relative to the up-down articulation hub 304, the articulation brake inner housing 307 rotates relative to the articulation brake position detent 308 and the up-down articulation brake caliper 310. Upon rotation in a first direction, pins (318) extending from the up-down articulation brake inner housing 307 towards the up-down articulation brake caliper 310 engage angled surfaces 360 of the up-down articulation brake caliper to axially move the articulation brake caliper 310 towards the articulation brake spring 309 to compress the articulation brake spring 309. Upon rotation in a second direction, the pins (move angled surface 360 to allow the articulation brake spring 309 to force the up-down articulation brake caliper 310 towards the articulation brake friction disc 311 and the up-down articulation brake outer housing 312 to compresses the articulation brake friction disc 311 between the up-down articulation brake caliper 310 and the up-down articulation brake outer housing 312. This contact between the stationary (relative to the reusable hand-piece) up-down articulation brake caliper 310, the articulation brake friction disc 311, and the up-down articulation brake outer housing 312 results in resistance of the up-down articulation knob 313, which is supported by the up-down articulation brake outer housing 312, rotating relative to the reusable hand-piece 200.

As rotation of the up-down articulation brake position detent 308 is fixed relative to the housing of the reusable hand-piece via the up-down articulation brake hub 304, rotation of the articulation brake inner housing 307 relative to the housing also rotates the articulation brake inner housing 307 relative to the up-down articulation brake position detent 308. The up-down articulation brake position detent 308 may be arranged to retain the up-down articulation brake inner housing 307 in one or more positions. For example, the detent may retain the up-down articulation brake inner housing in a first position in which braking resistance is applied via up-down articulation brake friction disc interaction between the up-down articulation brake caliper and the up-down articulation brake outer housing and/or in a second position one in which braking resistance is not applied. The brake inner housing and/or detent may be arranged for retaining the up-down articulation brake inner housing in any of a plurality of positions to provide an adjustable level of rotational resistance to the articulation control (e.g., articulation control knobs).

Extending through the center of the up-down articulation brake hub is an up-down articulation shaft 303. The up-down articulation shaft 303 has a first end 370 rotationally fixed relative to the up-down articulation brake outer housing. A second end 372 of the up-down articulation shaft 303 communicates with the rack assembly of the single-use shaft assembly. For example, the second end of the up-down articulation shaft may be attached to the up-down articulation pulley/pinion such that rotation of the up-down articulation shaft rotates the up-down articulation pulley/pinion. In this way, rotation of the up-down articulation knob rotates the up-down articulation brake outer housing which rotates the up-down articulation shaft which rotates the up-down articulation pulley/pinion which actuates the up-down articulation wire(s).

Regarding left-right articulation, the structure and operation of the left-right articulation knob and brake assembly is the same as the up-down articulation knob and brake assembly with the exception that rotation of the left-right articulation brake hub 317 is fixed relative to the housing 440 of the single-use shaft assembly and that such rotation is fixed via a left-right articulation brake shaft 301 extending through the left-right articulation shaft 302. Similar to the up-down articulation knob and brake assembly, the left-right articulation knob 314 is supported by a left-right articulation brake outer housing 315 which contains an articulation brake friction disc 311, a left-right articulation brake caliper 316, an articulation brake spring 309, an articulation brake position detent 308 and an articulation brake inner housing 307.

Extending through the left-right articulation brake knob 314 and the left right articulation brake outer housing 315 is the left-right articulation shaft 302. The left-right articulation shaft 302 has a first end 380 rotationally fixed relative to the left-right to articulation brake outer housing 315 and a second end 382 engaging the pulley/pinion assembly of the single-use shaft assembly (e.g., the second end rotationally fixed to the left-right articulation pinion of the rack assembly) such that rotation of the left-right articulation brake knob rotates the left-right articulation brake outer housing which rotates the left-right articulation brake shaft which in turn rotates the left-right articulation pulley/pinion which in is turn translates the left-right articulation wire(s).

The left-right articulation brake shaft 301, which extends through the left-right articulation shaft 302, has a first end 390 rotationally fixed to left-right articulation brake hub and a second end 392 rotationally fixed relative to the housing of the single-use shaft assembly when the reusable hand-piece assembly is attached to the single-use shaft assembly. Similar to the operation of the up-down articulation knob and brake assembly, rotation of the articulation brake inner housing relative to the left-right articulation brake caliper moves the left-right articulation brake caliper towards or away from the left-right articulation brake outer housing and the brake friction disc positioned therebetween. The left-right articulation brake knob 319 is rotationally fixed to the left-right articulation brake inner housing 307 such that rotation of the left-right articulation brake knob rotates the left-right articulation brake inner housing relative to the left-right articulation brake hub 317, the left-right articulation brake caliper 316, and the left-right articulation brake position detent 308. Accordingly upon engagement of the left-right articulation brake, the left-right articulation brake caliper and brake friction disc resist rotation of the left-right articulation brake outer housing and left-right articulation knob relative to the left-right articulation brake shaft and the single-use shaft assembly.

The second end of the left-right articulation brake shaft may be arranged to slidably engage with the housing of the single-use shaft assembly and rotationally fix the left-right articulation brake shaft relative thereto. Positioned circumferentially around the left-right articulation brake shaft is the left-right articulation shaft with its second end arranged to engage the left-right articulation pulley/pinion cooperating with the left-right articulation wire(s). And, positioned circumferentially around the left-right articulation shaft is the up-down articulation shaft having its second end arranged to engage the up-down articulation pulley/pinion associated with the up-down articulation wire(s).

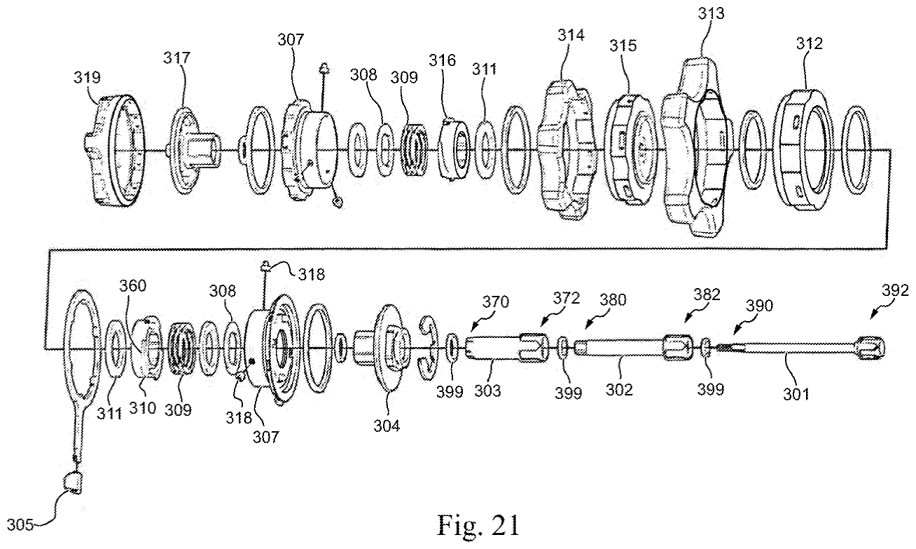

As shown in FIG. 21, the articulation knob and brake assembly may include a plurality of O-rings 399 between the various components when assembled.

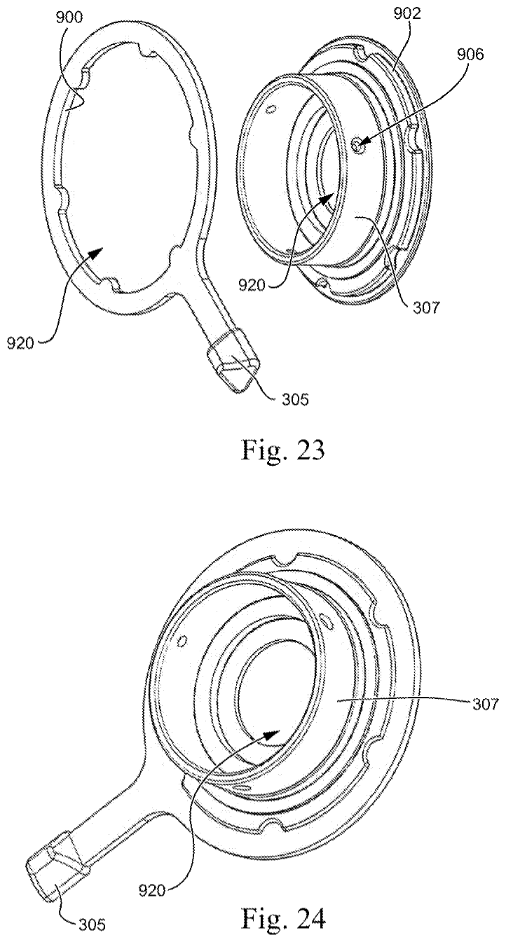

With reference now to FIGS. 23 and 24, the up-down articulation brake lever can have a surface 900 configured to mate with a surface 902 of the up-down articulation inner housing such that rotation of the up-down articulation brake lever rotates the up-down articulation brake inner housing relative to the housing of the reusable hand-piece assembly. Preferably, the geometry of surface 900 interferes with the geometry of surface 902 during rotation. The up-down articulation brake inner housing can define one or more apertures 906 arranged to receive transverse pins 318 that engage the angled surfaces 360 of the up-down articulation brake caliper 310. Additionally, an inner surface 910 of the up-down articulation brake inner housing can include recesses or protrusions arranged to engage detent spring (not shown). The up-down articulation brake lever and the up-down articulation brake inner housing define an aperture 920 for receiving the up-down articulation hub.

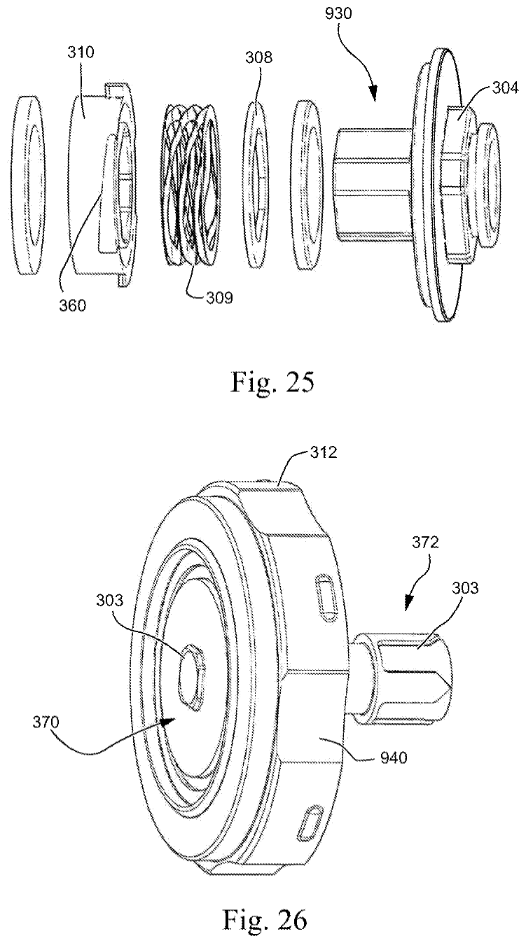

Turning now to FIG. 25, the up-down articulation hub 304 has a portion 930 that extends through the up-down articulation brake position detent 308 and the up-down articulation brake caliper 310. Preferably portion 930, the up-down articulation brake position detent 308, and/or the up-down articulation brake caliper have a cross-sectional geometry that fixes the up-down articulation brake detent, the up-down articulation brake caliper, and/or the up-down articulation brake hub from rotation relative to one another.

Turning now to FIG. 26, the up-down articulation brake outer housing 312 is shown engaged with and rotationally fixed relative to the up-down articulation shaft 303. The up-down articulation brake outer housing has a portion 940 arranged to rotationally fix the up-down articulation knob thereto (e.g., via recesses arranged to receive protrusions of the up-down articulation knob or vice versa).

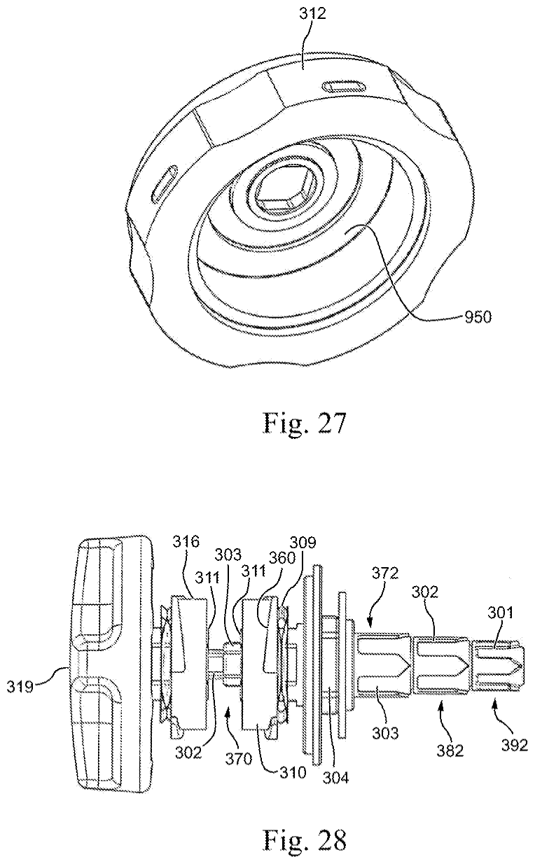

Turning now to FIG. 27, the inside of the up-down articulation brake outer housing is shown as having an inner friction surface 950 arranged to engage the up-down articulation brake friction disc when the up-down articulation brake is engaged.

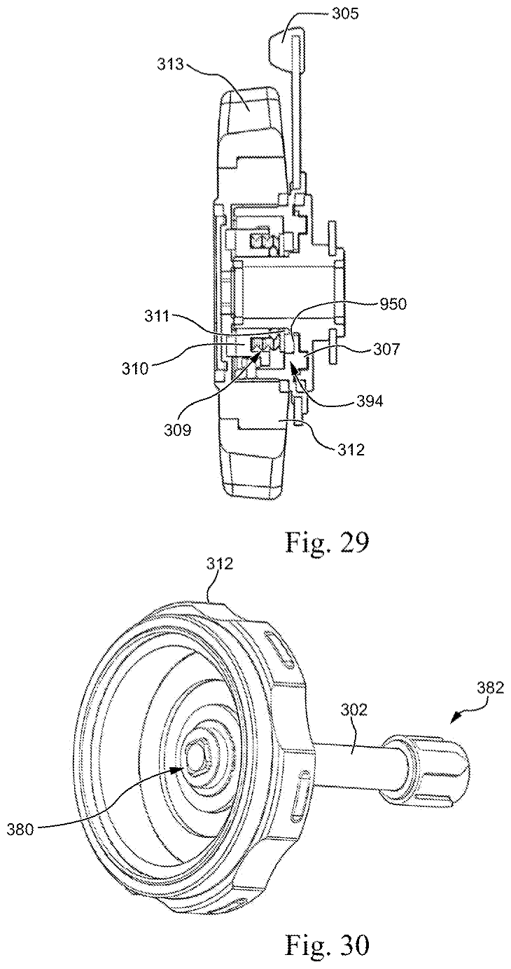

Turning now to FIG. 28, when the up-down articulation brake lever is rotated relative to the housing of the reusable hand-piece, the up-down articulation brake inner housing rotates with the up-down articulation brake lever and moves pins extending from the apertures of the up-down articulation brake inner housing relative to the up-down articulation brake caliper. The pins move along the sloped surfaces of the up-down articulation brake caliper to translate the up-down articulation brake caliper along the up-down articulation hub in cooperation with the up-down articulation brake spring. When configuring the up-down articulation brake from a disengaged configuration to an engaged configuration, the up-down articulation brake caliper forces the up-down articulation brake friction disc against the up-down articulation brake outer housing (compressing the up-down articulation brake friction disc). When configuring the up-down articulation brake from an engaged configuration to a disengaged configuration, the up-down articulation brake caliper moves away from the up-down articulation brake housing and the up-down articulation brake friction disc and compresses (e.g., further compresses) the up-down articulation brake spring. FIG. 29 shows the up-down articulation brake in the disengaged configuration with a gap 394 between the up-down articulation brake friction disc and the inner friction surface 950 of the up-down articulation brake outer housing.

The up-down articulation brake inner housing may have detent recesses or protrusions to engage one or more protrusions or recesses of the up-down articulation brake position detent. Such an arrangement may provide securement of the up-down articulation brake lever in one or more positions and/or provide a tactile and/or audible indication of the up-down articulation brake entering and/or leaving the one or more configurations or positions.

The left-right articulation knob and brake assembly work in a similar fashion to the up-down articulation knob and brake assembly with the exception of the left-right articulation brake hub being rotatably secured relative to the housing of the endoscope assembly by a left-right articulation brake shaft extending centrally through the left-right articulation shaft and the up-down articulation shaft.

With reference to FIG. 30, the left-right articulation brake outer housing and the left-right articulation shaft are shown assembled and rotationally fixed relative to one another.

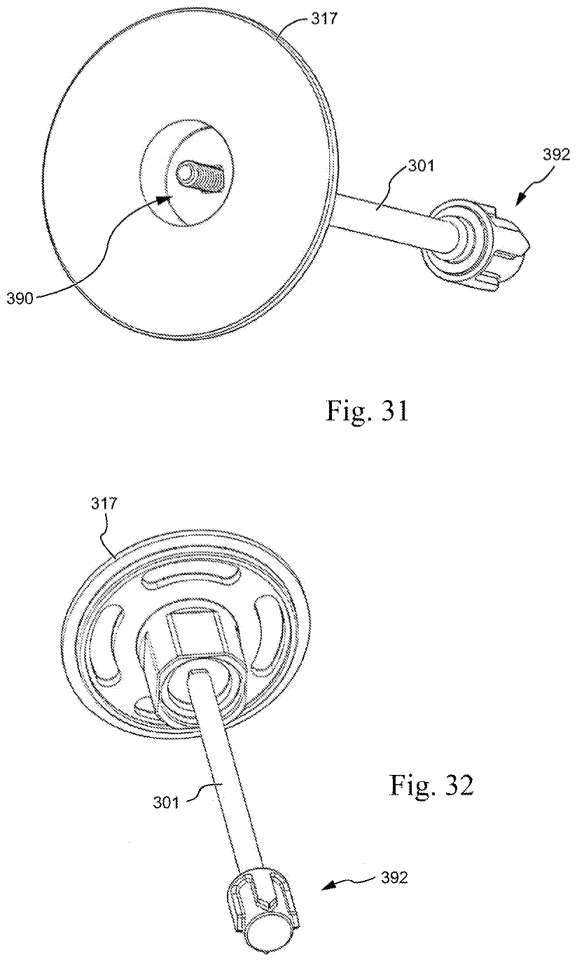

FIGS. 31 and 32 illustrate the left-right articulation brake shaft 301 in the left-right articulation brake hub 317. As can be seen along the left-right articulation brake shaft 301 has a first end 390 received within and rotationally fixed to the left-right articulation brake hub 317. A second end 392 of the left-right articulation brake shaft is arranged to engage the housing 240 of the single-use shaft assembly during attachment of the reusable hand-piece assembly and the single-use shaft assembly and rotationally fixe the left-right articulation brake shaft relative to the single-use shaft assembly. Accordingly, when the reusable hand-piece assembly is attached to the single-use shaft assembly, the left-right articulation brake hub is rotationally fixed relative to the housing of the single-use shaft assembly.

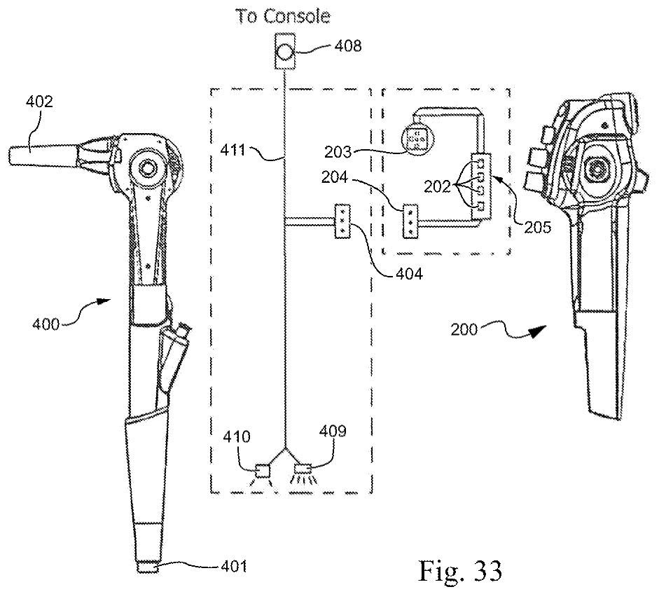

Turning now to FIGS. 33-35, exemplary wiring diagrams of the endoscope assembly are provided. The reusable hand-piece can include an electronics module 205, such as a circuit board, supporting and/or electrically connected to one or more of the control switches 202 and/or navigation switches 203 of the reusable hand-piece. Electrically connected to the electronics module is a connector 204 for electrically connecting the reusable hand-piece to the single-use shaft assembly when the reusable hand-piece and single-use shaft assembly are attached to one another.

Turning to the single-use shaft assembly, the single-use shaft assembly includes a corresponding electrical connector 404 configured to electrically connect the single-use shaft assembly with the reusable hand-piece when the two are attached. The electrical connector of the single-use shaft assembly can communicate with an optical sensor module 408, an optical sensor 409, and/or an LED 410 via conductors 411 of the single-use shaft assembly. The single-use shaft assembly may include conductors extending to the console (e.g., through the umbilical 402), such as to transmit images and/or control data to the console.

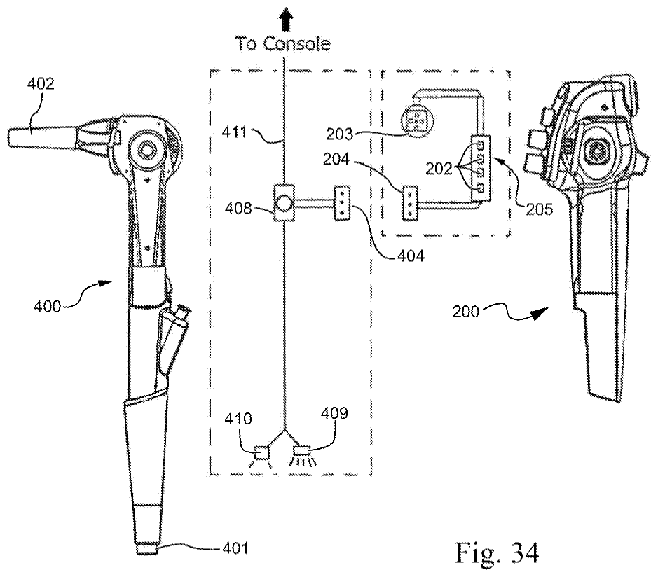

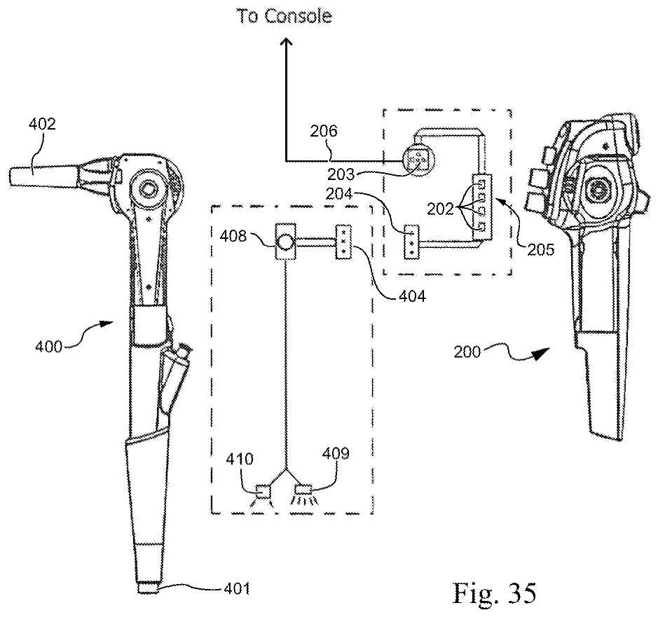

FIGS. 33 and 34 illustrate embodiments in which a tubing and/or electrical connector 402 (e.g., umbilical) extends from to the single-use shaft assembly to the console. While not limited to the arrangements of FIGS. 33 and 34, those figures also illustrate the optical sensor module 408 may be positioned outside of the housing of the single-use shaft assembly (e.g., FIG. 33) or inside of the housing of the single-use shaft assembly (e.g., FIG. 34). FIG. 35 illustrates an embodiment in which a tubing and/or electrical connector (e.g., umbilical) including electrical conductors 206 extends from the reusable hand-piece assembly to the console.

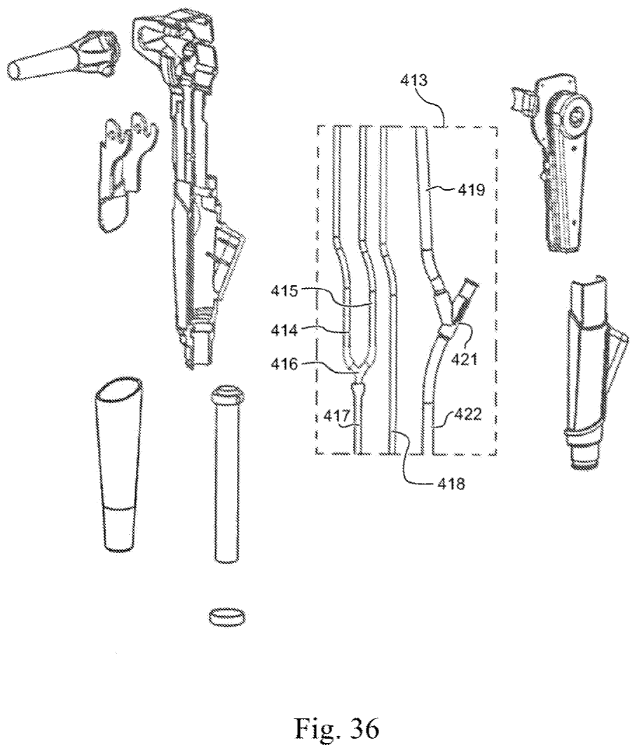

FIG. 36 illustrates an exploded view of the single-use shaft assembly and exemplary fluid circuits 413 thereof. As mentioned above, the single-use shaft assembly includes a shaft for insertion into the body of a patient and may include a tubing and/or electrical connector 402 (e.g., “umbilical”) extending to the console. Within the single-use shaft assembly may be a camera flush fluid channel 414, an insufflation fluid channel 415, a tissue irrigation fluid channel 418, a biopsy/section fluid channel 419, and/or a sample port fluid channel 422. One or more of the fluid channels may be combined (e.g., combined camera flush and insufflation channel 417) and/or connected such as by a T fitting 416 or Y fitting 421, so as to reduce the number of working lumens extending through the insertion shaft of the single-use shaft assembly.

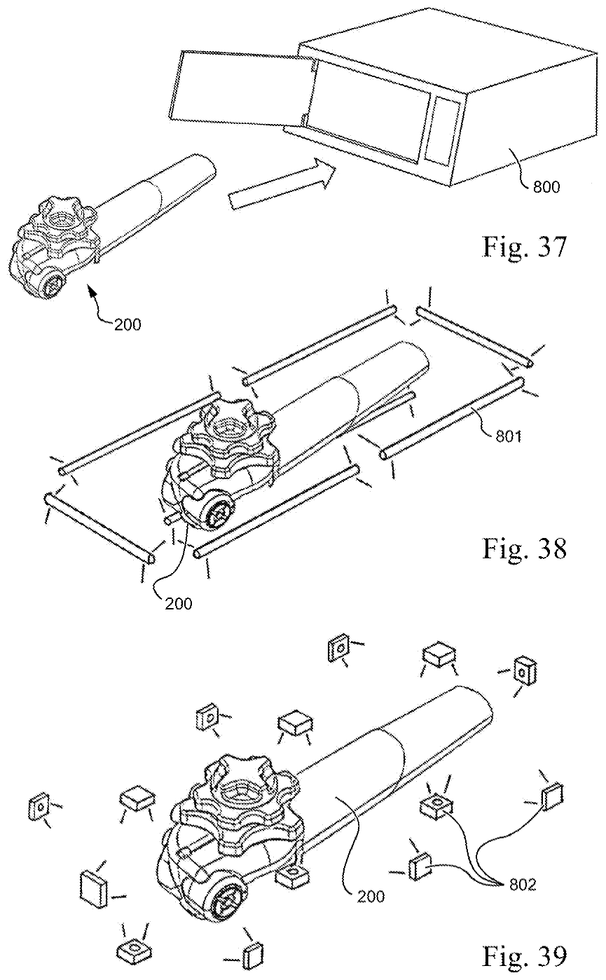

Turning now to FIGS. 37 through 39, FIG. 37 illustrates devices and methods useful for sterilizing the reusable hand-piece assembly. FIG. 37 illustrates an autoclave device 800 suitable for heating the reusable hand-piece assembly to a sufficient temperature to sterilize the reusable hand-piece assembly. FIG. 38 illustrates sterilization of the reusable hand-piece assembly using ultraviolet radiation. Such ultraviolet radiation may be generated by quartz lamps 801. Turning to FIG. 39, sterilization of the reusable hand-piece assembly may be accomplished by ultraviolet radiation via light emitting diodes (LEDs) 802.

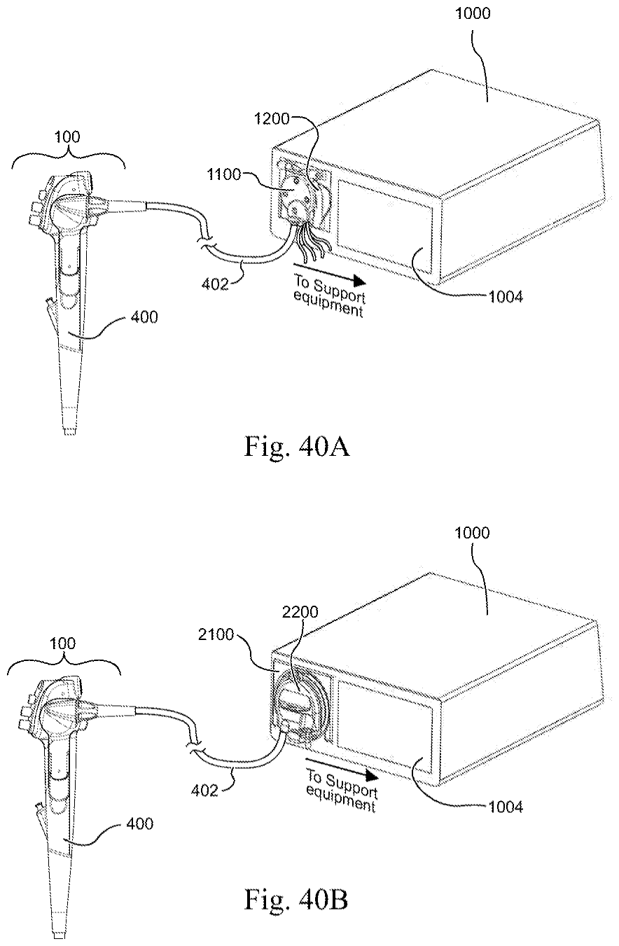

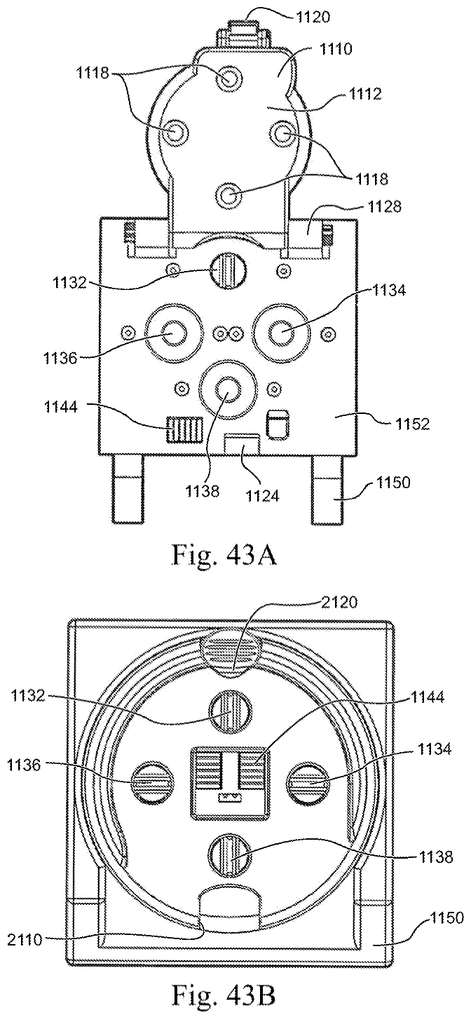

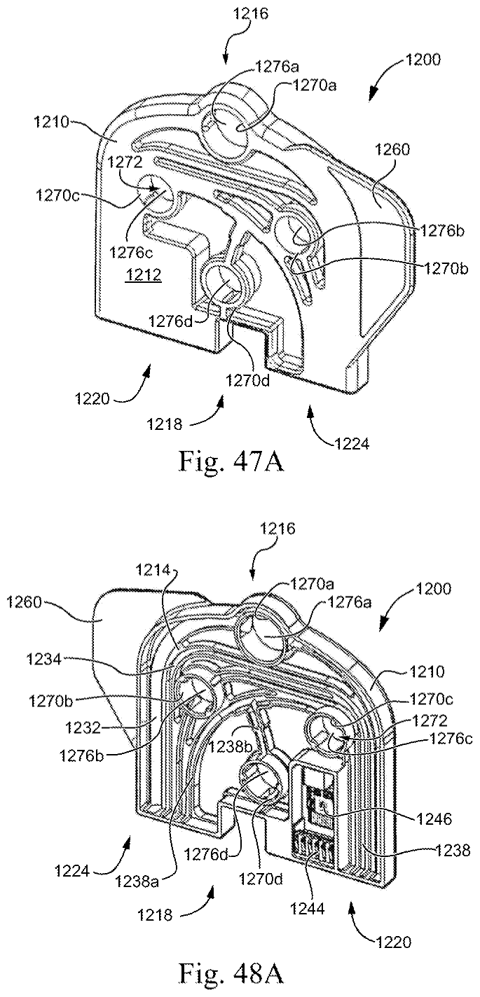

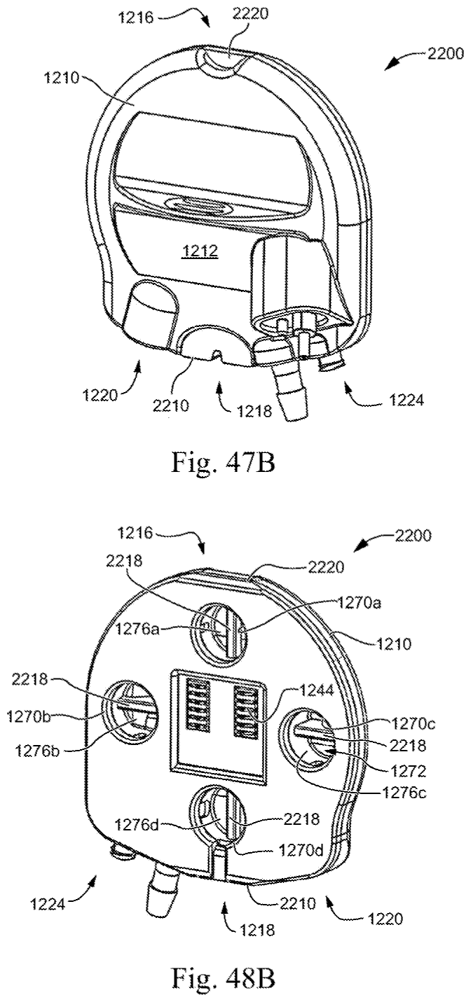

The present disclosure further provides a system comprising an endoscope assembly, a cartridge 1200 and a console 1000. The Figure A series for FIGS. 40-50 will be referenced first. FIG. 40 generally illustrates the system operationally connected for use, preferably by a medical professional. The system may include the cartridge 1200 inserted into a control valve assembly 1100 of the console 1000. The cartridge 1200 can be connected to the endoscope 100 by the umbilical 402. For instance, the cartridge 1200 may be connected to a single-use shaft assembly 400 of the endoscope by one or more fluid tubes and/or electrical conductors.

The cartridge 1200 may be connected to support equipment. The support equipment may include an irrigation pump, an insufflator, and/or a vacuum pump (e.g., stand-alone or central). The cartridge may be positioned between the endoscope and the support equipment.

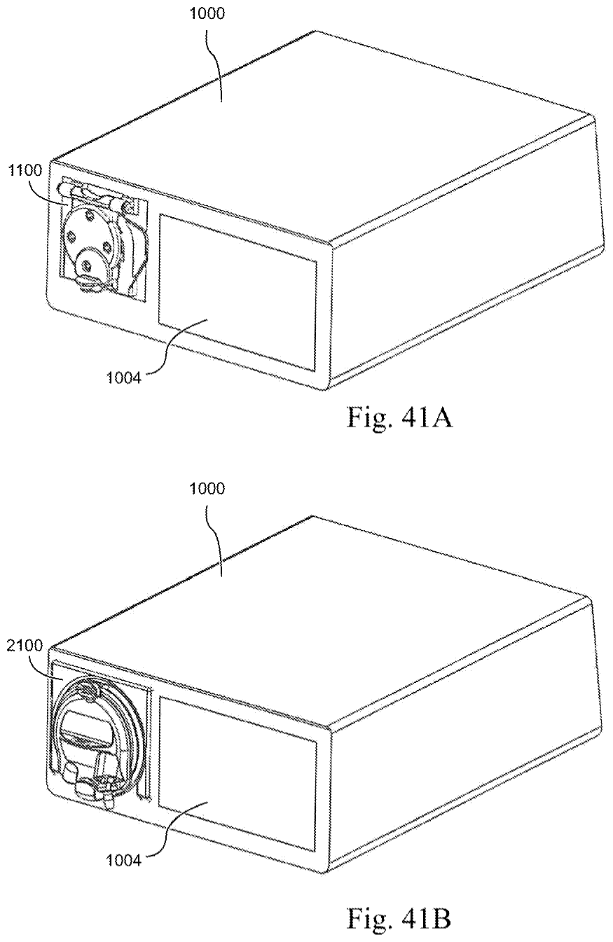

Turning to FIG. 41, the console 1000 is shown in greater detail. The console 1000 may include a user interface 1004 allowing the user to alter the various variables of the endoscope system. In one embodiment, the user interface 1004 may be a touchscreen integrated into an electric visual display. In another embodiment, the user interface could include a keyboard, mouse, trackball, and/or touch sensitive pointing device, etc.

The console 1000 may include a control valve assembly 1100 for receiving and actuating upon a cartridge. The console 1000 may provide electrical power, fluids and/or fluid pressure, vacuum pressure, and/or send and/or receive electrical signals with the endoscope assembly. The console 1000 may include a monitor (not shown) for visualizing signals received from the endoscope assembly such as a video feed from a camera at the distal end of the single-use shaft assembly.

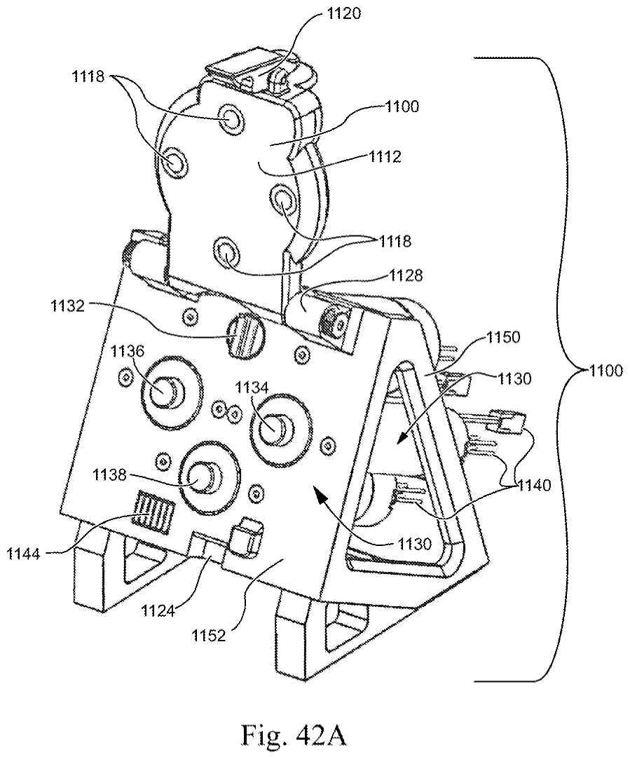

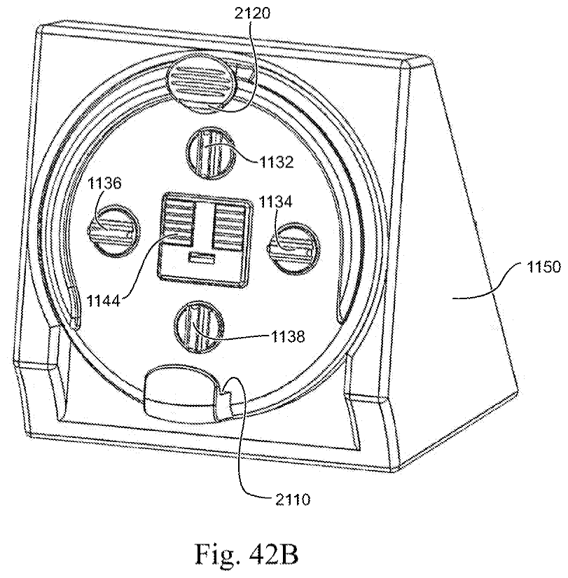

With reference to FIGS. 42-45, the control valve assembly 1100 is shown in greater detail. The control valve assembly 1100 may be used to selectively actuate an actuatable portion of fluid paths of the cartridge. The control valve assembly 1100 may include a door 1110, actuators 1130 and a support structure 1150 comprising a first surface 1152 and a second surface 1154.

The control valve assembly door 1110 may include a first surface 1112, a second surface 1114 and a side surface 1116. The first surface of the door can include a set of anvils 1118 for providing counter force to the actuators 1130 when selectively closing fluid paths of the cartridge 1200. In one embodiment, the first surface 1112 of the door includes four anvils 1118.

The door 1110 may include a latch 1120 capable of engaging a keeper 1124 of the control valve assembly. The latch may be a lever latch. When a cartridge 1200 is retained in position on the control valve assembly 1100 with the door 1110 and the latch 1120 is securing the door 1110 in a closed position, the door 1110 may provide compressive force on the cartridge. Advantageously, such compressive force may ensure electric spring fingers of an electric connector of the cartridge and/or electrical spring fingers of an electric connector 1144 of the control valve assembly engage with electrical contacts of the other to provide electrical communication between an electrical conductor of the endoscope and/or cartridge and electrical conductor of the console.

The door 1110 can be attached to the first surface of the support structure using a mechanical bearing 1128, such as a hinge capable of rotating the door between an open and closed configuration.

The actuator 1130 may be a linear actuator or rotational actuator. The actuator may be a solenoid. Although as few as one actuator is appreciated by the applicant, in preferred embodiments the control valve assembly includes four actuators. The actuators are arranged to actuate valve portions of fluid paths in the cartridge to selectively open and/or close the fluid paths.

The valve portions of the fluid paths may comprise any acceptable valve. For example, the valve portion may comprise a petcock. The valve portion may also comprise a portion of tubing arranged for compression (e.g., a pinch valve) between the anvil on the first surface of the door and the actuator (e.g., solenoid) of the control valve assembly. The valve portions may serve as a valve for any number of fluid functions of the endoscope, such as a suction valve associated with actuator 1132, a camera flush valve associated with actuator 1134, an insufflation valve associated with actuator 1136, and/or an insufflation vent valve associated with actuator 1138.

The actuators 1130 of the control valve assembly include electrical connectors 1140 to be received by the console and electrically connect the actuators to the console 1000. When connected to the console 1000, the console 1000 can provide electrical power and user inputted commands to the actuator 1130 to open and/or close the fluid pathways of the cartridge 1200.

The first surface 1152 of the support structure 1150 includes the keeper 1124 described above for receiving the latch 1120 and securing a cartridge 1200 to the control valve assembly 1100 with the door 1110. The latch assembly may be used to ensure that the cartridge is inserted in the correct configuration.

The cartridge 1200 may include an identifier readable and/or writable by the control valve assembly. The identifier may include information regarding the type of cartridge and/or usage information (e.g., whether the cartridge has been used before). Preferably, the control valve assembly is configured to read and process the identifier of the cartridge (e.g., to determine the type of cartridge and/or whether the cartridge has been used previously). The identifier may be readable and/or writable through an/the electrical connector of the cartridge and/or through other means (e.g., RFID, optics, and/or mechanical contact).

The first surface 1152 of the support structure 1150 may include the electrical connector 1144 for forming electrical connections to the electrical connector of the cartridge. The electrical connection between the control valve assembly of the console and the cartridge may allow the console to receive images and control data (valve/actuator and/or image data) passed from the endoscope through the cartridge to the console. The electrical connection may allow the console to supply power to the light-emitting diode (LED) of the endoscope assembly.

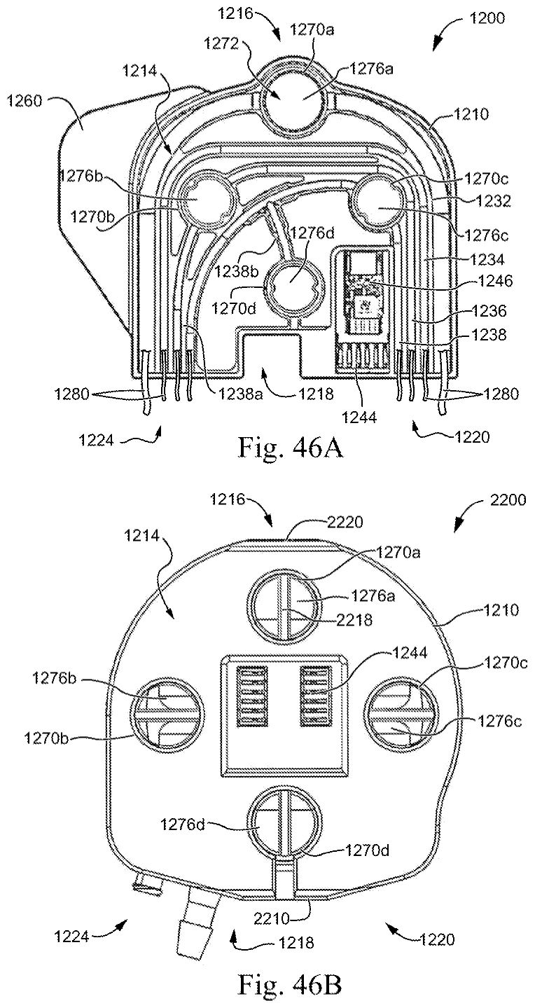

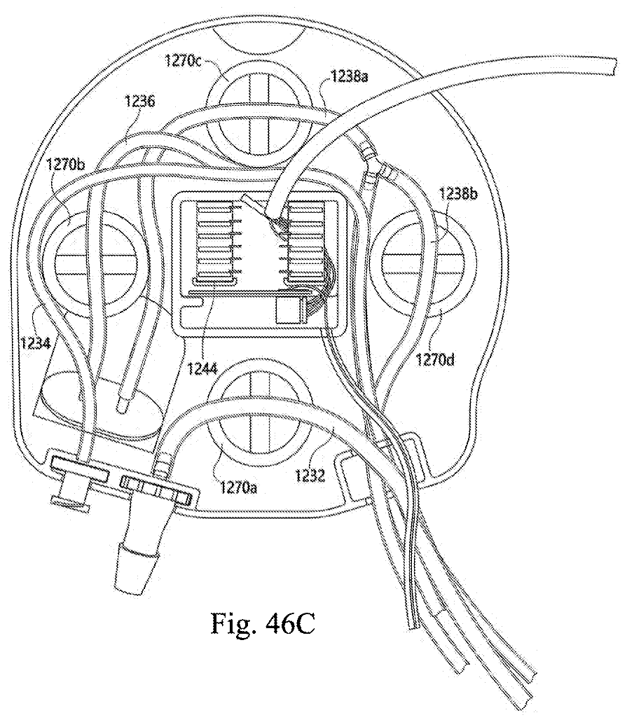

Turning to FIGS. 46-48, an exemplary cartridge 1200 is shown in greater detail. The cartridge can be a single-use cartridge. The single-use cartridge can negate the need for the use of permanent valve bodies contained within a reusable endoscope and/or the console. This advantageously eliminates the need to sterilize reusable valves after every use.

The cartridge 1200 has a housing 1210. The housing 1210 can include a first side 1212 and a second side 1214, as well as a top side 1216 and bottom side 1218. The housing 1210 further comprises a first fluid side 1220 and a second fluid side 1224 allowing for fluid to travel in and out of the cartridge housing. The housing 1210 may define one or more fluid pathways, fluid connector, and/or electrical connectors. For example, cartridge housing can include electrical connector 1244 in electrical communication with an electrical conductor of the endoscope and optionally an optical sensor module 1246 that contains optical sensor calibration data or a unique identifier for the endoscope. The housing 1210 may also include a removal tab 1260 for easy removal of the cartridge 1200 from the control valve assembly 1110 after a single use.

The housing may include a first fluid path 1232, a second fluid path 1234, a third fluid path 1236 and a fourth fluid path 1238. The fourth fluid path 1238 can comprise a main portion 1238a extending from the first fluid side 1220 to the second fluid side 1224 of the cartridge housing 1210 and a branch portion 1238b for venting out of the bottom side 1218 of the cartridge. A fluid path's cross-section may be larger than the other fluid path cross-sections. The fluid paths can generally form a U-shape in the cartridge housing. The first, second, third, and fourth fluid paths preferably are not be in fluid communication with each other within the cartridge.

The housing may define windows 1270a-d. In one embodiment, the housing includes four windows. The windows may be defined by inner surfaces of the housing. The inner surface may create a generally circular opening in the housing. The circumference of one window may be larger than the other windows to receive a larger fluid path cross section.

The windows 1270a-d can be configured to receive an actuator from the console into a chamber 1272 defined by the inner surface. As described above, the actuator may pass through the window and/or contact an anvil on the door of the control valve assembly of the console to create a pinch valve with the door. When the cartridge is positioned on the control valve assembly and the door is closed, the windows align with the anvils on the door and the actuators of the control valve assembly. The windows may be arranged to define a geometric shape. For instance, as illustrated, the windows may define a diamond.

The fluid paths of the housing are preferably defined by a flexible membrane. The flexible membrane can be flexible medical tubing, thermoplastic sheets, etc. The material of the housing is preferably more rigid than the tubing material. In an even more preferred embodiment, the flexible tubing is made of polyvinyl chloride.

The first fluid path 1232 can be a suction path, the second fluid path 1234 can be an irrigation path (optionally operated by a foot switch), the third fluid path 1236 can be a camera flush path, and/or the fourth fluid path 1238 can be an insufflation path. The insufflation path can include a main portion 1238a extending from the first fluid side to the second fluid side of the housing. It may further include a branch portion 1238b for venting gases out of the patient.

Fluid paths of the cartridge may include at least one valve portion 1276. However, the second fluid path may not include a valve portion. The fourth fluid path may include two valve portions 1276c, 1276d. As described above, the fluid path may be defined by a flexible membrane (e.g., tubing). The valve portion may comprise the flexible membrane described above.

The first fluid path 1232 may include a first valve portion 1276a that extends through a first window 1270a defined by the housing 1210. The first valve portion aligns with a first actuator of the console. The first actuator is actuatable to selectively close and/or open the first valve portion of the first fluid path by extending into the first window.

The second fluid path 1234 may be free of a valve portion. Preferably, the fluid path free of a valve is the irrigation pathway. The irrigation pathway may include a pump optionally operated by a foot switch.

The third fluid path 1236 may include a valve portion 1276b that extends through a window 1270b defined by the housing 1210. The valve portion aligns with an actuator of the console. The actuator is actuatable to selectively close and/or open the valve portion of the third fluid path by passing through the window.

The fourth fluid path 1238 may include a main portion 1238a and a branch portion 1238b as described above. The main portion of the fourth fluid path of the cartridge may include a valve portion 1276c that extends through a window 1270c defined by the housing. The valve portion aligns with an actuator of the console. The actuator is actuatable to selectively close and/or open the valve portion of the main portion of the fourth fluid path by passing through the window.

The branch portion of the fourth fluid path of the cartridge may include a valve portion 1276d that extends through a window 1270d defined by the housing. The valve portion aligns with an actuator of the console. The actuator is actuatable to selectively close and/or open the valve portion of the branch portion of the fourth fluid path by passing through the window.

As described above, one or more actuators may be solenoids configured to extend into the windows of the cartridge housing and/or contact the anvils on the door of the control valve assembly creating a pinch valve when closed.

The housing 1210 may further include an electrical connector 1244 for connecting to the electrical connector 1144 of the control valve assembly 1100. The electrical connector of the cartridge may be spring finger electrical contacts configured to contact a corresponding number of flat pad electrical contacts on the control valve assembly of the console or vice versa. The electrical connector of the cartridge housing may pass images and control data provided by the endoscope to the console. The electrical connector of the housing may pass power from the console to the LED used by the endoscope in the patient's body.

Data and/or power electrical conductors may extend from the cartridge to the endoscope through the umbilical.

The cartridge may include connectors 1280 on the first fluid side 1220 and/or the second fluid side 1224 for any and/or all of the fluid pathways defined in the cartridge housing. The connectors can extend beyond the cartridge (e.g., free ends of the continuous tubing extending beyond the cartridge with a fitting (e.g., a luer fitting) and/or be a portion of the cartridge.

The cartridge may optionally incorporate an optical sensor module 1246 that incorporates camera calibration data and a unique optical sensor module identifier for the single-use endoscope and cartridge. The unique optical sensor module identifier allows the console to recognize when the same single-use endoscope and single-use cartridge have been used, thus, eliminating the potentially dangerous use of an unsterilized endoscope. The unique identifier may further identify what type of endoscope is being connected to the console. The unique identifier may be incorporated into a printed circuitry board.

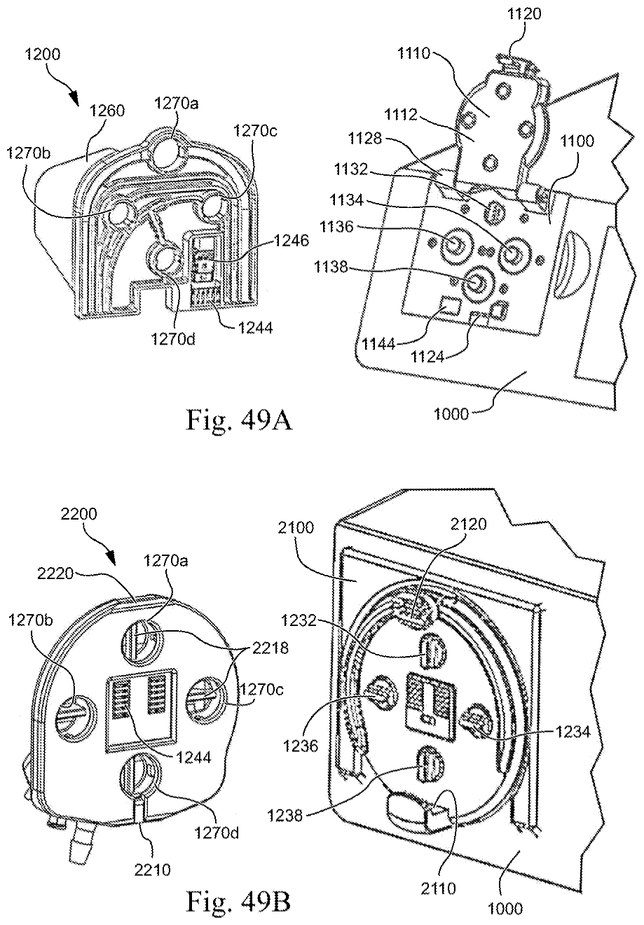

FIG. 49 further illustrates the cartridge separated from the control valve assembly. As described above, user aligns the windows of the cartridge with the actuators of the control valve assembly for insertion. Once inserted, the door may swing over the cartridge to secure it between the first surface of the support structure and first surface of the door. As described above, the latch is received by the latch keeper. The latch may be a lever latch, hasp latch, toggle latch, cam lever latch, etc. In one embodiment, the latch provides compression pressure against the cartridge securing it to the console. In preferred embodiments, this allows the spring fingers of the electrical connector of the cartridge to engage a corresponding flat pad electrical connector of the control valve assembly portion of the console, or vice versa, thus creating contact upon placement of the cartridge against the console and eliminating the need to connect one or more discrete electrical connectors.

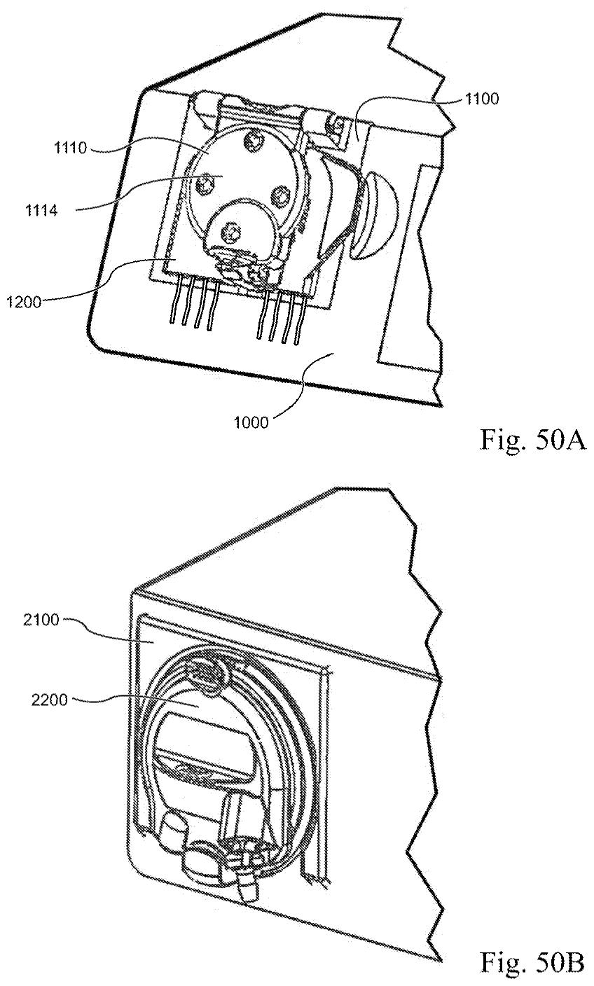

Turning to FIG. 50, a closer view of the cartridge inserted into the control valve assembly of the console is shown. As can be seen, the cartridge is inserted between the first surface of the support structure of the control valve assembly and the first surface of the door. The first fluid side 1220 of the cartridge is shown as loose tubing that continues into the umbilical and to the single-use shaft assembly of the endoscope (not shown)

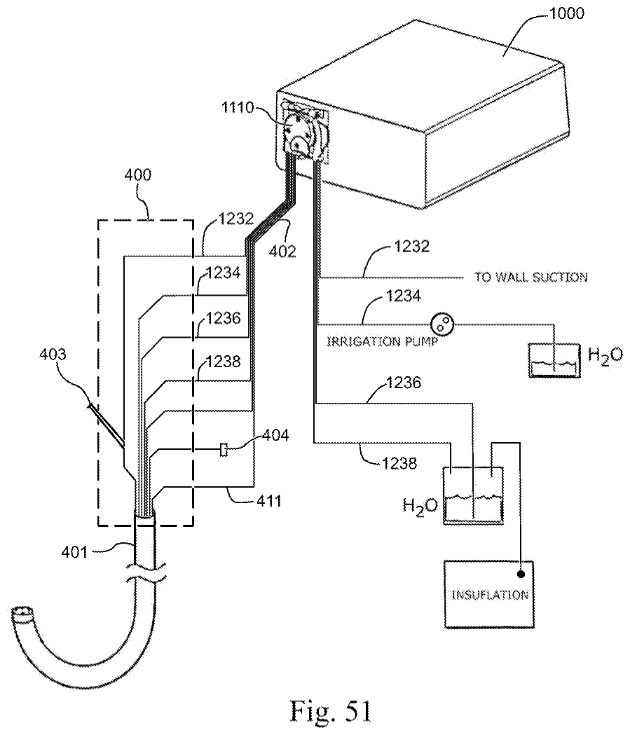

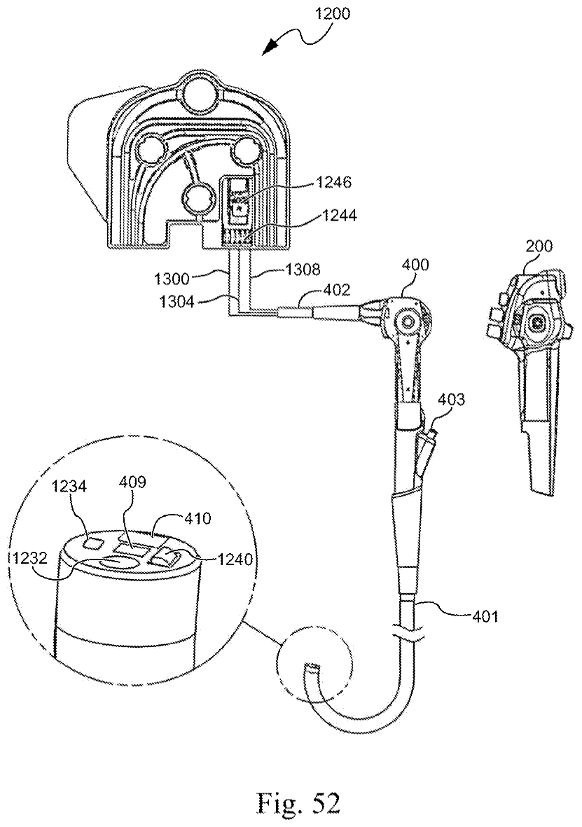

FIGS. 51-52 illustrate schematic views of an endoscopic system. The electrical connections between the cartridge and the single-use shaft assembly are schematically demonstrated. The electrical connectors 411 may include an image data conductor, control data conductor, and/or LED Power conductor. The image data conductor and/or control data conductor can allow for information to be passed from the endoscope to the cartridge and on to the console without any connections made by a user other than attaching the cartridge to the console. This layout advantageously decreases the number of direct connections and loose wires necessary to operate the endoscope system.

The insertion tube 401 may include an optical sensor module for providing readable information by the console and/or project an image on a monitor such as a video feed from the end of the insertion tube. This video feed can be infrared, thermal or visible light. The insertion tube 401 may include an LED wire for providing power to an LED at the end of the insertion tube.

The insertion tube may include fluid paths discussed herein defined by tubes, such as a suction tube 1232, tissue irrigation tube 1234, camera flush tube 1236, and/or insufflation tube 1238. The irrigation tube allows the user to clear the visual field of any debris during use of the instrument. Often, this involves the removal of excess blood so the place of interest may be visualized. The suction tubing allows for the aspiration of particulate matter or various secretions from inside the patient's body. The camera flush tube 1236 and insufflation tube 1238 may be combined by a Y fitting. Various combinations of the tubing are envisioned by the applicant to minimize the number of tubes required in the insertion tube.

The second fluid side of the cartridge may be connected to support equipment. The support equipment may include an irrigation pump, an insufflator, and/or a vacuum pump (stand-alone or central). The cartridge may provide for fluid communication between the endoscope and the support equipment.

The single-use shaft assembly can include an insertion tube, an umbilical 402 connected to the cartridge and therefore the console, an access port for selectively accessing a lumen of the single-use shaft assembly (e.g., for taking a sample such as a biopsy) and/or for delivering a fluid through a lumen of the single-use shaft assembly, and/or an electrical connector for electrically coupling the single-use shaft assembly to the reusable hand-piece.

The umbilical can extend from the cartridge to the single-use shaft assembly of the endoscope. Extending within the umbilical to the distal tip of the insertion tube are several fluid paths and/or electrical wires. The fluid paths may include paths for suction 1232, tissue irrigation 1234, camera flush 1236, and/or insufflation 1238. The fluid pathways can be free of valves from the distal tip of the endoscope to the end of the umbilical. The fluid pathways may include valves only within the boundary of the cartridge.

Preferably, one or more fluid paths are defined by monolithic tubing extending from the single use shaft assembly to the cartridge. More preferably, the monolithic tubing extends through the cartridge and out of the second fluid side of the cartridge.

The camera flush fluid pathway and insufflation fluid pathway may be combined by a T-fitting in the single-use shaft assembly into a combined camera flush and insufflation fluid pathway 1240. This allows the pressure from insufflation to be used to push water from a water source through the camera flush fluid pathway and out the distal end to clean the camera lens of debris collected during a procedure.

The umbilical may further include an image data conductor 1300, control data conductor 1304, and/or an LED power conductor 1308. The image data conductor and/or LED power conductor may extend through the single-use shaft assembly to the distal tip of the insertion tube. The image data conductor may allow the transmission of the image data taken from the camera back to the console. The LED power conductor may provide power from the console to the LED at the distal tip of the insertion tube, thus providing illumination for the procedure. The control data conductor allows the communication of control data from the controls of the reusable handpiece to the cartridge which, in turn, allows communication to the console.

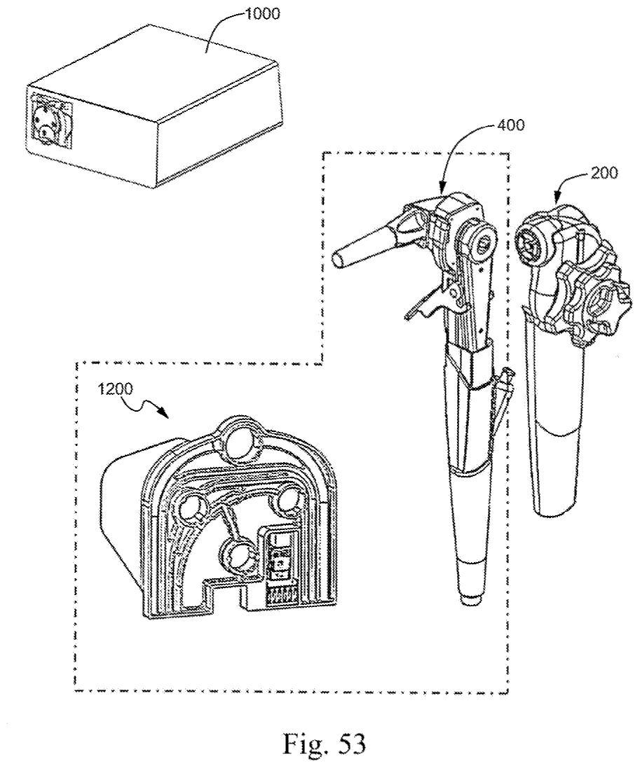

Turning to FIG. 53, the single-use shaft assembly, umbilical, and cartridge are sterilely sealed within medical packaging, such as a medical tray. The cartridge and single-use shaft assembly may be connected using the umbilical within the packaging but, in some instances, may be subsequently connected by a medical professional.

The cartridge 2200 and control valve assembly 2100 illustrated in the ‘B’ series of FIGS. 40-50 and FIG. 46C is similar to the cartridge 1200 and control valve assembly 1100 illustrated the ‘A’ series of FIGS. 40-50, discussed above. The ‘B’ series of FIGS. 40-50 and FIG. 46C illustrates a control valve assembly 2100 and cartridge 2200 that eliminates the need for a door by, for example, relocating the anvils from the door to the cartridge. The elimination of the door reduces the number of steps required to both load and unload a fluid control cartridge. The elimination of the door reduces the number of components and interfaces that could become soiled by dirt or fluids and require cleaning. Additionally, the elimination of the door reduces the risk of unintended contact with the console by a person moving around the unit—which could potentially dislodge the console from a cart and/or cause discomfort to the person.

A doorless cartridge 2200 and control valve assembly 2100 is illustrated in the ‘B’ series of FIGS. 40-50. The control valve assembly 2100 may include a ledge surface 2110 and a latch 2120. The cartridge 2200 may include a ledge surface 2210 and a latch receiving portion 2220. In alternate examples, the control valve assembly may include the latch receiving portion and the cartridge may include the latch. When the cartridge is received by the control valve assembly the latch contacts the latch receiving portion to secure a first end of the cartridge to the control valve assembly. Separately, and opposite of the latch and latch receiving portion, the ledge surface of the cartridge and the ledge surface of the control valve assembly contact each other to secure a second end of the cartridge to the control valve assembly.

The cartridge may include an anvil 2118 similar to the anvil discussed in the control valve assembly with a door example above. The anvil 2218 is configured to provide counter force to the actuators when selectively closing fluid paths of the cartridge. Preferably, the anvils 2218 are be located in windows defined in the cartridge.

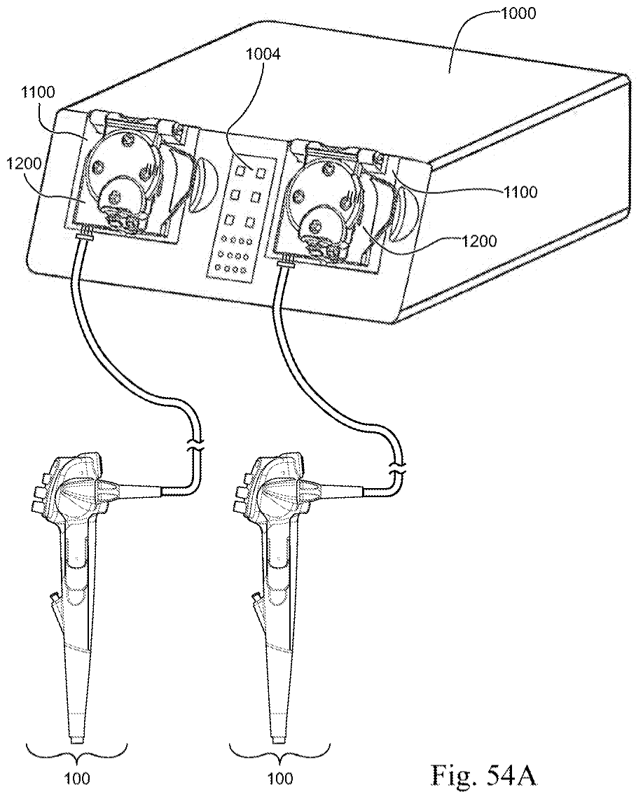

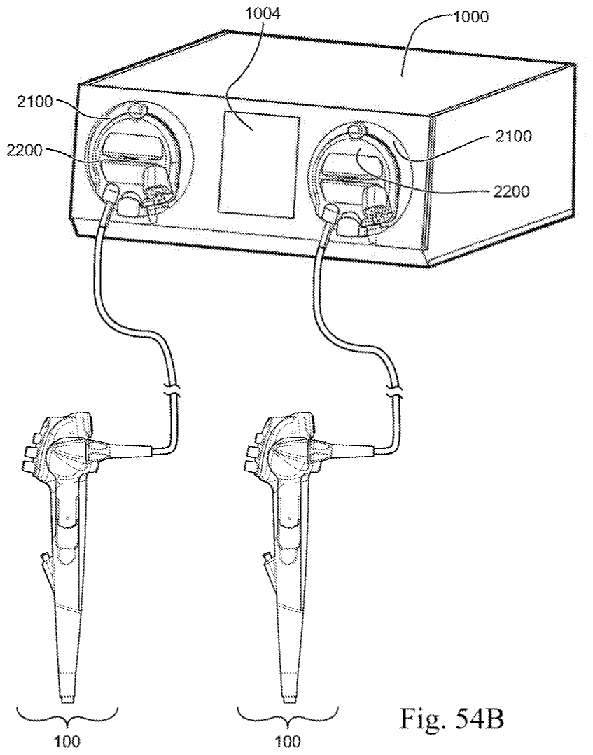

FIGS. 54A and 54B illustrate another example of the console with a first cartridge, a second cartridge, a first endoscope and a second endoscope. The first cartridge and the second cartridge may be securely inserted into a first control valve assembly and a second control valve assembly of the console, respectively. Although the console is illustrated with the first and second control valve assembly, the console may have any number control valve assemblies each capable of receiving an independent cartridge. The first control valve assembly may receive the first cartridge and the second control valve assembly may receive the second cartridge. It is not necessary for every control valve assembly to receive a cartridge during every medical procedure.

As illustrated, each cartridge may be connected to a separate set of support equipment and an independent medical device, such as an endoscope. Non-limiting examples of the types of endoscopes that may be used include, gastroscopes, colonoscopies, sigmoidoscopes, duodenoscopes and cholangiopancreatoscopes. Depending on the procedure being performed, the endoscopes may be the same type of endoscope or two or more different types of endoscopes, such as a duodenoscope and a cholangiopancreatoscope used during an Endoscopic Retrograde Cholangiopancreatography (ERCP) procedure. During use, the first endoscope and the second endoscope may be secured together by a strap, zip tie, etc. The handle of the first endoscope may be configured to mate with a handle of the second endoscope. A shaft of the second endoscope may inserted and extended through the length of a shaft of the first endoscope, such as a cholangiopancreatoscope inserted within a shaft of a duodenoscope. This advantageously allows a single user to control one or more endoscopes at a time during a procedure.

The duodenoscope may be known as the first endoscope or mother scope. The smaller cholangiopancreatoscope may be known as the second endoscope or daughter scope. During operation, the larger mother scope may be inserted through the patient's mouth and directed to the duodenum. The daughter endoscope is then able to operate with the patient's bile duct. The daughter endoscope may have its own fluid flow paths, LED and/or camera.

The first control valve assembly and the second control valve assembly are configured to operate independently and/or in a main/secondary communication configuration. The first or mother endoscope may be the main controller and the second or daughter endoscope may be the secondary controller. In this arrangement, the controls of the main endoscope may control the fluid functions (e.g., image controls and/or LED) of one or more secondary endoscopes. The main endoscopes may control the function of the fluid paths of the secondary endoscopes by providing an electrical signal to the second control valve assembly to selectively actuate an actuator as described herein (e.g., through one or more cartridges connected to the console). The first endoscope may further control a function of the second endoscope (e.g., operation of the LED and/or camera) by providing an electrical signal from the first endoscope to alter a condition of the second control valve assembly (e.g., alter an electrical signal provided by the second control valve assembly to the cartridge of the second endoscope). For instance, the control valve assembly may provide an electrical signal to the LED and/or the camera of the second endoscope based on the signal received from the first endoscope. This arrangement further allows for multiple endoscopes to be setup and utilized in a procedure at once, eliminating or reducing extensive setup time typically used to change one endoscope out for another endoscope and/or eliminating or reducing the need for additional equipment, such as multiple control consoles.

The control valve assemblies, endoscopes, cartridges, consoles, fluid paths, electrical conductors, functions, and operations described and illustrated herein apply equally to the embodiments of the ‘A’ series of FIGS. 40-50 and the embodiments of the ‘B’ series of FIGS. 40-50, with the exception the console in the ‘B’ series is doorless and the cartridge of the ‘B’ series includes a latch and/or latch receiving portion. Therefore, the discussion concerning the control valve assemblies, the endoscopes and cartridges can be any of the cartridge or endoscope embodiments herein and are applicable to both the ‘A’ series (with a door) and the ‘B’ series (doorless) arrangements.

Due to varying image sensors and the necessary fluid controls required by different types of endoscopes, the number and/or type of fluid paths and electrical conductors used in each cartridge may vary. Accommodations may be made for image sensor variability by the video processing unit that can be adapted to the specific type of optical sensor incorporated into the connected endoscope. The optical sensor type may be associated with a unique identifier communicated to the console through the cartridge by, for example, port control protocol or an electrical signal associated with the endoscope. In the case of fluid pathway control, the unique identifier may cause the console to turn off the functionality of certain actuators on the control valve assemblies. Further, depending on the type of endoscope connected, one or more fluid pathways can be eliminated from the cartridge entirely.

The console is illustrated with a touchscreen user interface. The user interface may be any suitable input/output device.

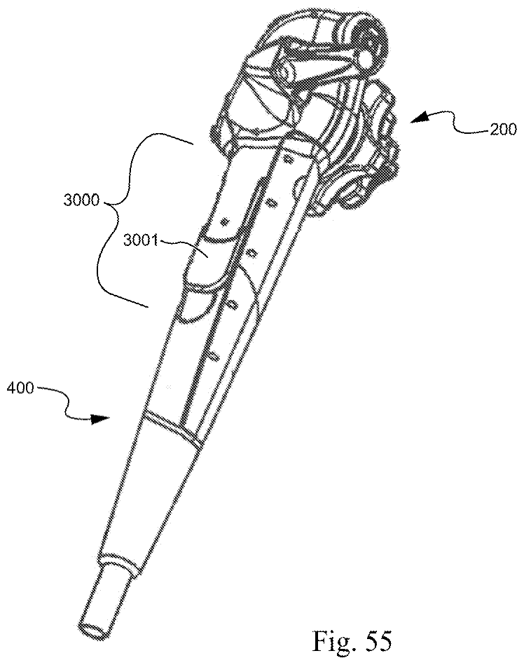

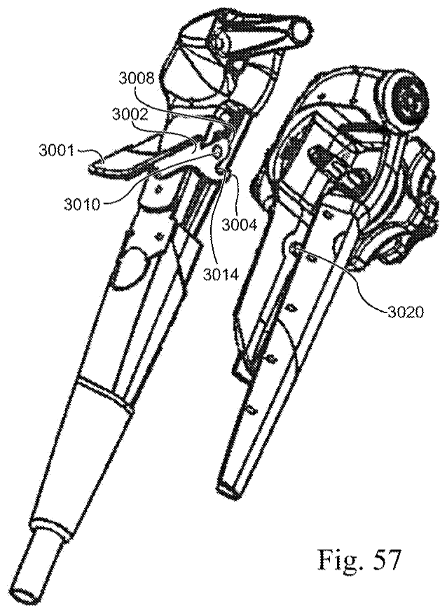

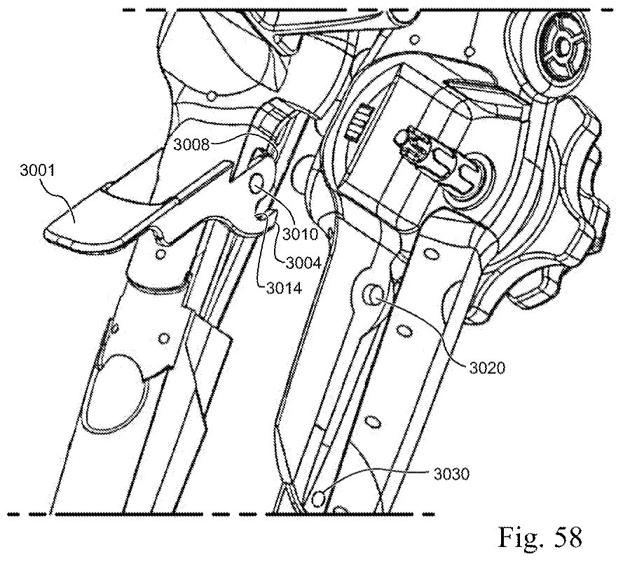

Turning to FIGS. 55-58, an endoscope assembly is illustrated with a coupler or selectively securing the reusable hand-piece assembly to the single-use shaft assembly. The coupler can include a first portion on the reusable hand-piece assembly and a second portion on the single-use shaft assembly, wherein the first and second portions of the coupler cooperate to hold the reusable hand-piece and the disposable shaft assembly together in the assembled configuration. The coupler can be arranged for actuation (e.g., to connect and/or to disconnect) without use of a hand tool (e.g., screwdriver). The coupler is actuatable to engage and/or disengage the reusable hand piece assembly from the single-use shaft assembly.

The current example of the endoscope assembly may be any of the examples including a reusable hand-piece assembly and a single-use shaft assembly discussed herein. Additionally, the endoscope assembly may be any endoscope including two or more separable pieces. As discussed in various places herein, the reusable hand-piece assembly and the single-use shaft assembly include a hand-piece housing and a single-use shaft assembly housing, respectively.

The coupler may be a latch 3000, such as a cam-style draw latch. The latch may be selectively configurable between a latched configuration and an unlatched configuration. In the latched configuration, the latch latches the housing of the hand-piece assembly to the housing of the shaft assembly. When configuring from the latched configuration to the unlatched configuration, the latch may apply a separating force to separate the housing of the hand-piece assembly and the housing of the shaft assembly.

The hand-piece assembly may be a reusable hand-piece assembly and the shaft assembly may be a single-use shaft assembly.

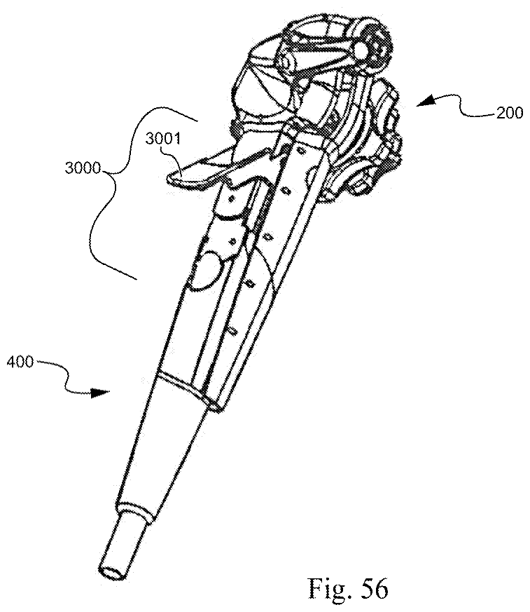

The latch may include a lever 3001. The lever may be pivotable between a first position in the latched configuration (demonstrated in FIG. 55) and a second position in the unlatched configuration (demonstrated in FIG. 56).

The latch may further include a latching surface 3004 and an unlatching surface 3008. In the latched configuration the latching surface retains the housing of the hand piece in proximity to the housing of the shaft assembly. When configuring to the unlatched configuration from the latched configuration the unlatching surface applies the separating force to separate the housings.

The at least two piece endoscope assembly may include a biasing member 3030 and a latch configurable between a latched configuration and an unlatched configuration, such as any latch disclosed herein. The biasing member applies a biasing force to bias the hand-piece assembly housing and the shaft assembly housing towards or away from one another. The latch may be configured to apply a force counter to the direction of force of the biasing member, and greater in magnitude than the force of the biasing member, to force the housings away from or towards one another. For example, the biasing member may be configured to bias the housings towards one another, and the latch may be configured to force the housings away from one another. In an alternative example, the biasing member may be configured to bias the housings away from one another, and the latch may be configured to force the housings toward one another. The biasing member may be a spring, a magnet and/or a pressure chamber, just to name a few non-limiting examples. The pressure chamber can be a piston cylinder (e.g., pneumatic), a compressible bladder, and/or a suction cup.

In greater detail, the latch may include a latch portion 3002 and a latch receiving portion 3020 (i.e., the first portion and the second portion). The housing of the reusable hand-piece assembly and/or the housing of the single-use shaft assembly may define the latch and latch receiving portion. For example, the latch may be part of the housing of the reusable hand-piece assembly while the latch receiving portion is part of the housing of the single-use shaft assembly. As another example, the latch may be part of the housing of the single-use shaft assembly while the receiving portion is part of the housing of the reusable hand-piece assembly.

The latch portion may include the latching surface, the unlatching surface and the lever. While in the latched configuration, the latching surface contacts the latch receiving portion to draw the single-use shaft assembly and the reusable hand-piece assembly together. While in the unlatched configuration, the latch receiving portion and the latching surface are not in contact.

The unlatching surface, advantageously, can aid in separating the single-use shaft assembly and reusable hand-piece assembly. While moving from the latched to the unlatched configuration, the unlatching surface contacts the latch receiving portion and applies a force to separate the reusable hand-piece assembly and the single-use shaft assembly.

The lever may control the movement of the latching surface and the unlatching surface. The lever may be freely movable between a first position in the latched configuration and a second position in the unlatched configuration. The lever may be pivotable around a latch pivot 3010 from the latched configuration to the unlatched configuration and/or vice versa. The latching surface and the unlatching surface may be located on opposing sides of the latch pivot. While in this structural configuration, during actuation, the latching surface and unlatching surface rotate around the latch pivot in opposing directions.

The latch portion may further include a seat portion 3014. The latching surface may be a curved surface sloping on one side into the u-shaped seat. The opposite leg of the u-shaped seat slopes upward transitioning to the unlatching surface. In this configuration, the latching surface and unlatching surface may be located on opposing sides of the seat portion.

When configuring the reusable hand-piece to the single-use shaft in the latched configuration, the lever may be pivoted around the latch pivot from a vertical unlatched first position angled away from the housing to the latched second position flush with the housing/handle. In these examples, the ability to grip the lever with the lever in the latched configuration indicates that the reusable hand-piece and the single-use shaft assembly are coupled to one another. The latch can be arranged to indicate when the reusable hand-piece and the single-use shaft are correctly couple to one another. In one example, the latch can provide an audible “click” when the reusable hand-piece and the single-use shaft assembly are correctly coupled to one another.

When the lever is manipulated into the latched position the latching surface is rotated toward and received by the latch receiving portion to connect the housing of the single-use shaft assembly to the housing of the reusable hand-piece assembly. While the latching surface is engaged with the latch receiving portion, the latching surface helps draw the reusable hand-piece and single-use shaft together. The latching surface is inserted into and through the latch receiving portion and rotated upward so that the latch receiving portion sits within the seat portion. At the same time, the unlocking surface may rotate away from the opposing housing piece, enabling the connection of the two housings.

To configure the reusable hand-piece and the single-use shaft assembly in the unlatched configuration, a user may pivot the lever away from the surface of the housing of the endoscope into the second unlatched configuration. Pivoting the lever vertically away from the housing may rotate the latching surface back through and out of the latch receiving portion. Simultaneously, the unlatching surface may counter-rotate in relation to the latching portion contacting the opposing housing piece. When pivoting the lever into the disconnected position, the unlatching surface assists in separating the housing of the reusable hand-piece assembly and the housing of the single-use shaft assembly. The unlatching surface is rotated to push away the opposing housing piece.

As described above, the latch can be biased with a biasing member, such as a spring, into the latching configuration that can hold the reusable hand-piece and single-use shaft together. The latching mechanism may further include one or more magnets that assist in holding and/or separating the reusable hand-piece to/from the single-use shaft assembly.

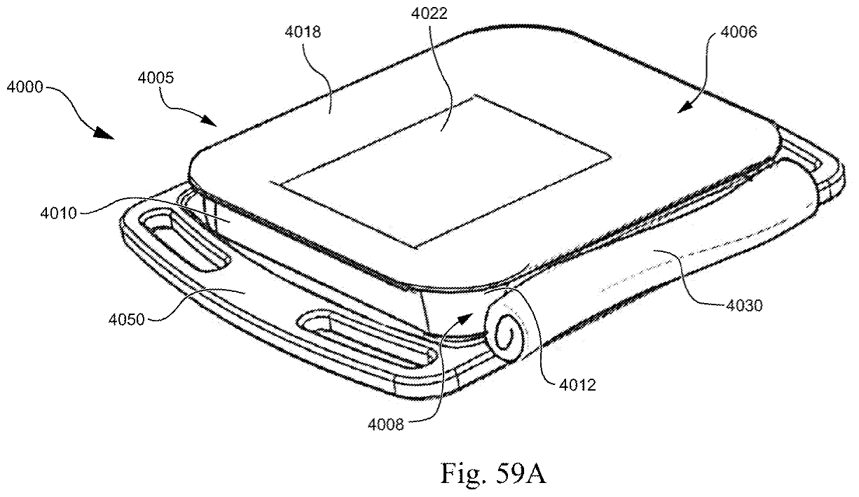

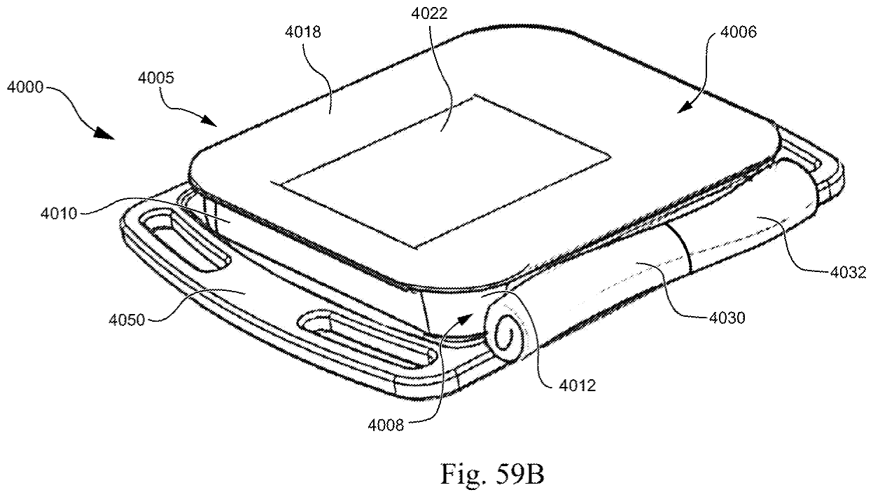

FIGS. 59A and 59B illustrates a packaging system 4000 for a medical product, such as an endoscope assembly or a portion of an endoscope assembly (e.g., a single-use endoscope assembly). The packaging system 4000 includes a container 4005 that includes a first portion 4006 for retaining the medical product and a second portion 4008 for supporting a bag 4030 for the biohazardous material that is created from use of the medical product and a bag 4032 for the reusable hand-piece retained within the first portion 4006. The first portion 4006 preferably includes a sterile portion for retaining the medical product in sterilely sealed condition. The second portion 4008 may be a sterile portion of packaging system 4000 or may be a non-sterile portion.

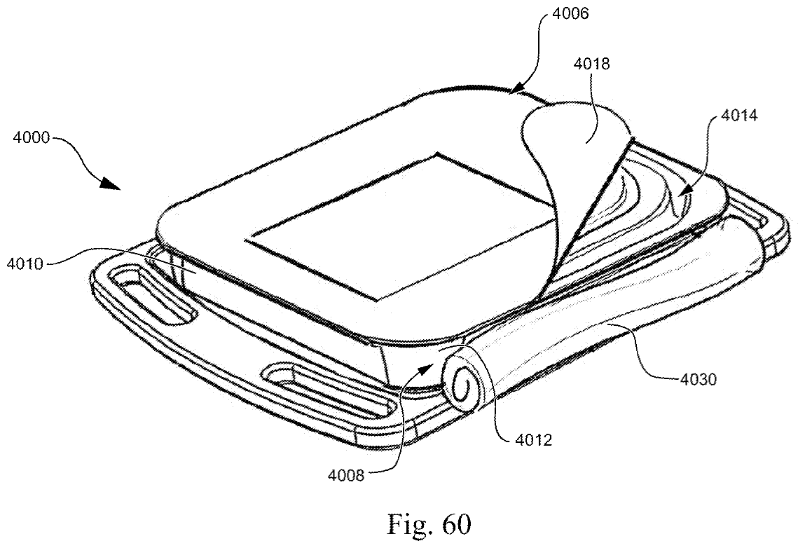

In the embodiment shown in FIG. 59, the container 4005 includes a tray 4010 that defines a recess 4014 (see FIG. 60) for receiving the medical product. In some embodiments, the tray 4010 may be manufactured by thermoforming and may be made of a recyclable material that allows the tray 4010 to be recycled after use. The recess 4014 is enclosed by a cover 4018, such as a peel-away cover that is formed by a Tyvek® sterile barrier, so that the medical product may be sterilely sealed within the recess 4014. A label 4022 may be placed on the exterior of cover 4018 to identify medical product within the recess 4014 of tray 4010 and to provide any other desired information to a user.

The second portion 4008 of the container 4005 may be positioned on the exterior of the first portion 4006 or may be contained within the first portion 4006. For example, the second portion 4008 may be an area within the first portion 4006 accessible after the cover 4018 is removed. As another example, the second portion 4008 may be located on an exterior surface of the first portion 4006 or tray 4010 (e.g., outside of the recess 4014). In the embodiment shown in FIG. 59, the second portion 4008 of the container 4005 includes an exterior side 4012 of tray 4010. The biohazard disposal bag 4030 is removably attachable to the container 4005 at this exterior side of tray 4010. As an example, the biohazard disposal bag 4030 may be removably attached to tray 4010 using adhesive, adhesive tape, hook and loop fasteners, and/or a perforated line (e.g., linear and/or curvilinear). Although shown in FIG. 59 as being attached to an exterior surface of tray 4010, in other embodiments, the biohazard disposal bag 4030 may be removably attached to an interior surface of tray 4010 within recess 4014.

In other embodiments, the biohazard disposal bag 4030 may include means for securing biohazard disposal bag 4030 to a surface in a medical examination room and/or operating suite, such as a cart, fixture, table, bed, and/or wall, just to name a few non-limiting examples. Such means may include adhesive, adhesive tape, hook and loop fastener, magnets, hooks, clamps, and clips. Such means may also include an aperture arranged to receive a protrusion (e.g., a hook) extending from the surface and/or vice versa. The means for securing the bag to a surface may be the same or different from the means for removably attaching the biohazard disposal bag to the second portion 4008 (e.g., the exterior of the tray 4010).

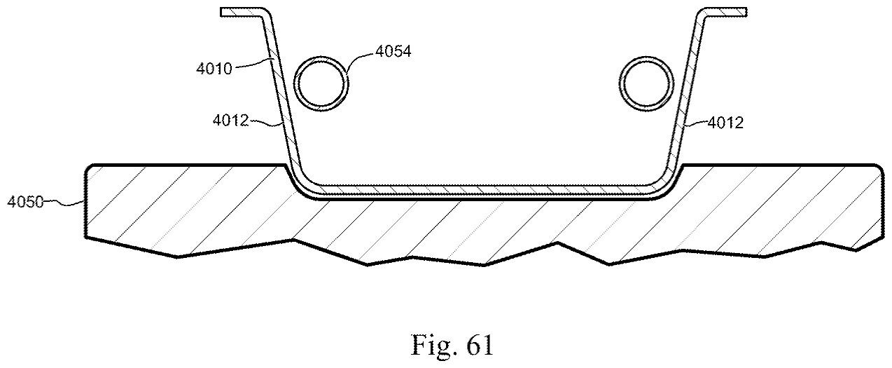

As shown in the cross-sectional view illustrated in FIG. 61, the packaging system 4000 may be supported by a surface 4050 of a cart or a roll stand that allows the packaging system 4000 to be easily moved to a desired location. This allows the packaging system 4000 to be moved within reaching distance of a doctor or a nurse that is performing a procedure on a patient so that the medical product within the packaging system 4000 can be easily accessed and easily disposed of after use. Additionally, FIG. 61 shows a possible curvature for the side walls of the tray 4010 to allow optimal space for the minimum bend radius of a insertion tube 4054 for a medical product inserted into the recess 4014 of tray 4010.

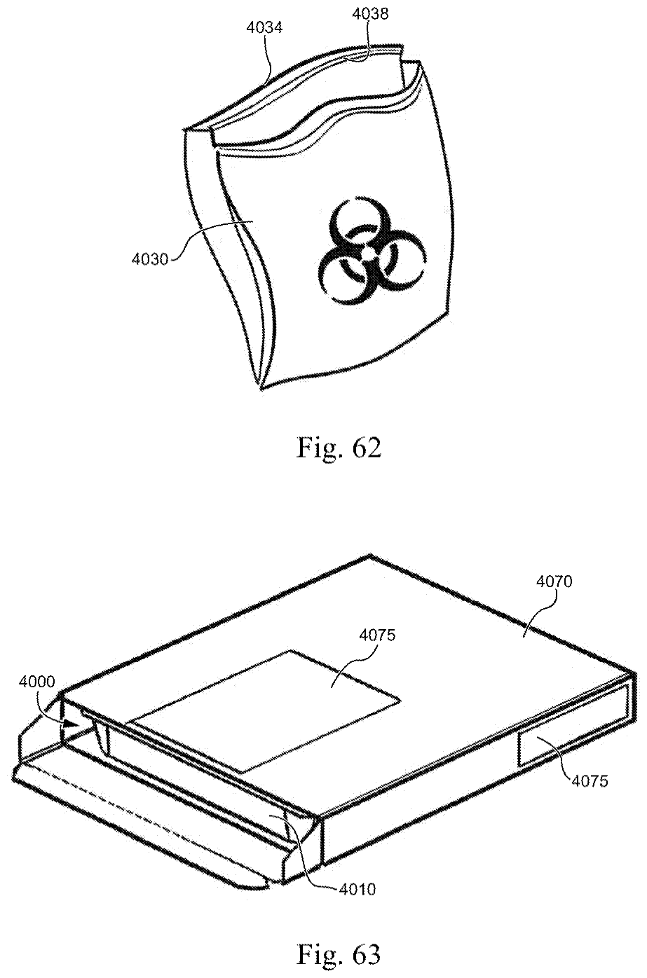

FIG. 62 illustrates an embodiment of the biohazard disposal bag 4030 when it is unrolled and ready to receive biohazardous waste. As shown, preferably the means for securing the biohazard disposal bag 4030 to a surface and/or the means 4038 for removably attaching the biohazard disposal bag 4030 at the second portion 4008 are spaced along at least 20% of a perimeter opening 4034 of the biohazard disposal bag. Advantageously, such an arrangement can aid in holding the perimeter opening 4034 of the biohazard disposal bag 4030 in an open configuration to allow easy insertion of the medical product within the biohazard disposal bag 4030 after the medical product has been used.

As shown in FIG. 63, the container 4005 and the biohazard disposal bag 4030 of the packaging system 4000 may be packaged within the same medical product box 4070 (e.g., a paper and/or cardboard box). Medical product box 4070 holds both the container 4005 and the biohazard disposal bag 4030 while packaging system 4000 is shipped to a medical professional for use and for storage by the medical professional before the medical product within the container 4005 is used. Although the term “box” is used, it should be recognized that the medical product box 4070 may be a cardboard box, paper packaging, a plastic wrap or plastic container, or any other suitable structure for holding the container 4005 and the biohazard disposal bag 4030.

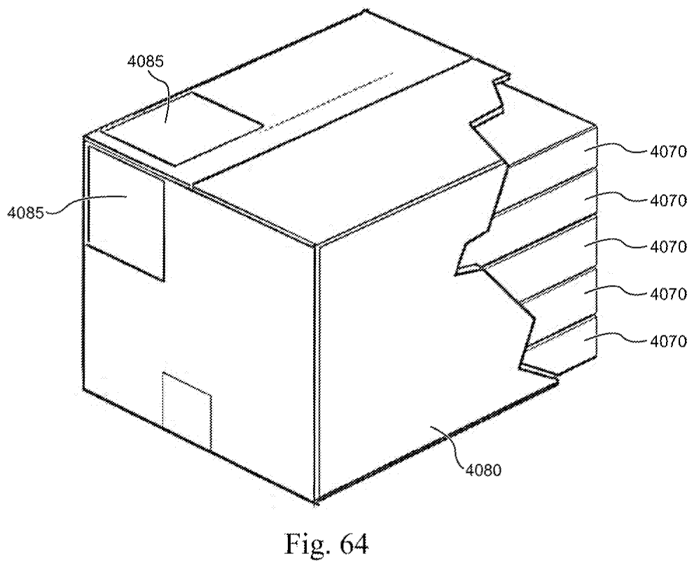

One or more medical product boxes 4070 may be positioned within a shipping box 4080 (e.g., a corrugated shipping box)(see FIG. 64). One or more labels 4075, 4085 having information pertaining to the medical product (e.g., product name, product dimensions, and/or instructions for use) may be positioned on the medical product box 4070 and/or the shipping box 4080.

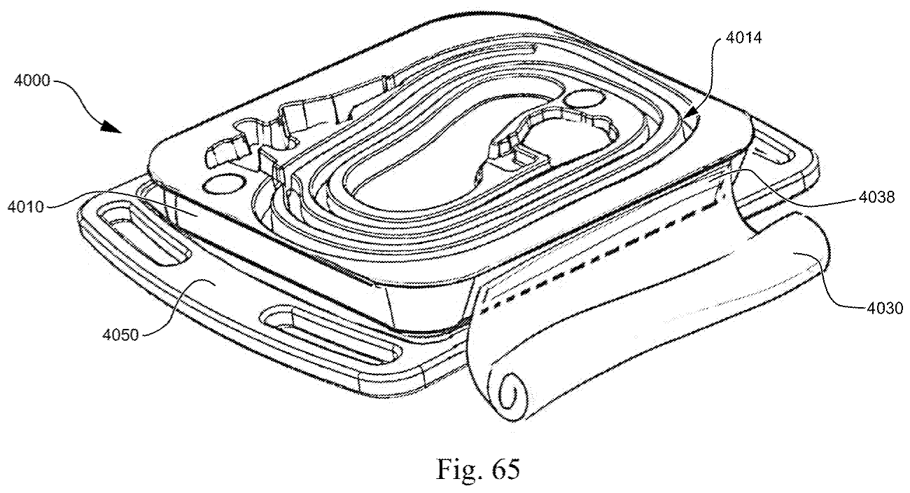

FIG. 65 shows a packaging system 4000 in which the medical product has been used so that the packaging system 4000 and the medical product is ready for disposal. The tray 4010 shown in this embodiment includes a cavity 4014 that is arranged to hold a one-time use endoscope. The endoscope is removed from the cavity 4014 of tray 4010 while being used for the desired procedure.

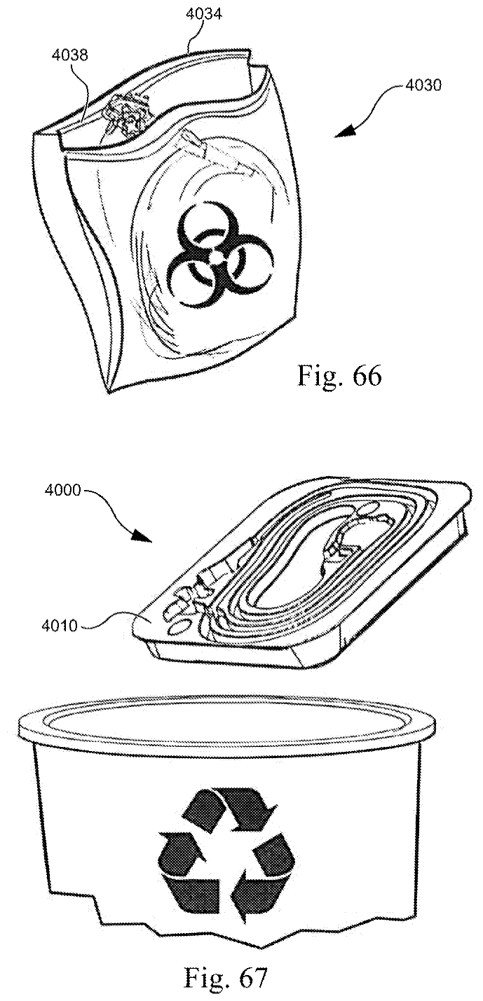

As shown in FIG. 66, after the procedure is completed, the used endoscope is disposed of by being placed within the biohazard disposal bag 4030. The biohazard disposal bag 4030 can be retained on the second portion 4008 of packaging assembly 4000 while the endoscope is placed within the biohazard disposal bag 4030. As described above, the means for securing the biohazard disposal bag 4030 to the second portion 4008 of the packaging system 4000 may be used to help keep the biohazard disposal bag 4030 while the endoscope is placed within the biohazard disposal bag 4030, so that no assistance is needed from an additional party.

In certain embodiments, one biohazard disposal bag 4030 may be used to dispose of the single-use shaft assembly. A second bag 4032 may be used to retain the reusable hand-piece.

After the medical product in its entirety or the single-use shaft assembly has been used and properly placed within the biohazard disposal bag 4030, the biohazard disposal bag 4030 may be detached from the second portion 4008 of the packaging system 4000 and disposed of in the proper method for biohazardous waste.

After the reusable hand-piece has been placed in the second bag 4032. The bag 4032 may be detached from the second portion 4008 of the packaging system 4000. The bag 4032 with the reusable hand-piece inside may be safely transferred for reprocessing. In some examples, the medical professional or someone associated with the medical professional may ship the bag 4032 including the reusable hand-piece back to the manufacturer for reprocessing/cleaning. Once the reprocessing operation is complete, the reusable hand-piece may be shipped back to the medical professional and/or the same practice. In other examples, the reusable hand-piece can be resold to a third party, depending on the agreement between the manufacturer and the original medical professional.

As long as the used medical product is properly placed within the biohazard disposal bag 4030 and does not come into contact with the packaging system 4000, the packaging system 4000 should not be contaminated and can be recycled after being separated from the biohazard disposal bag 4030 (see FIG. 67).

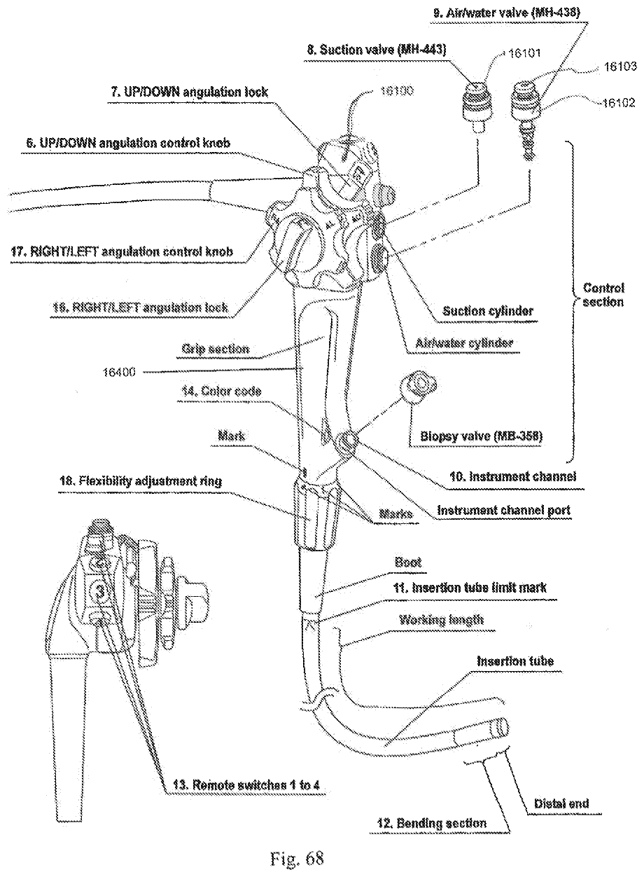

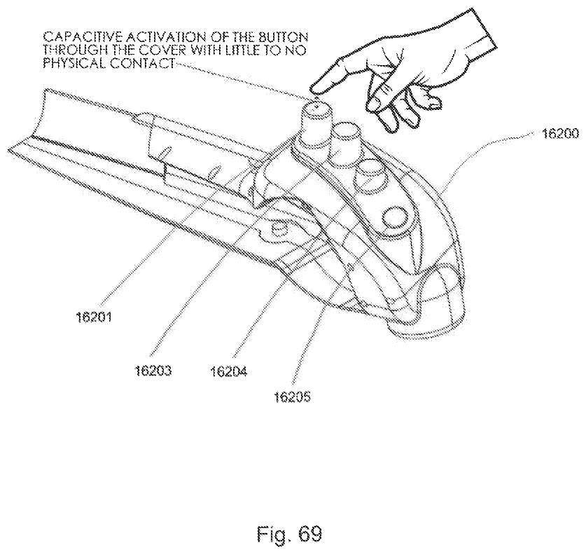

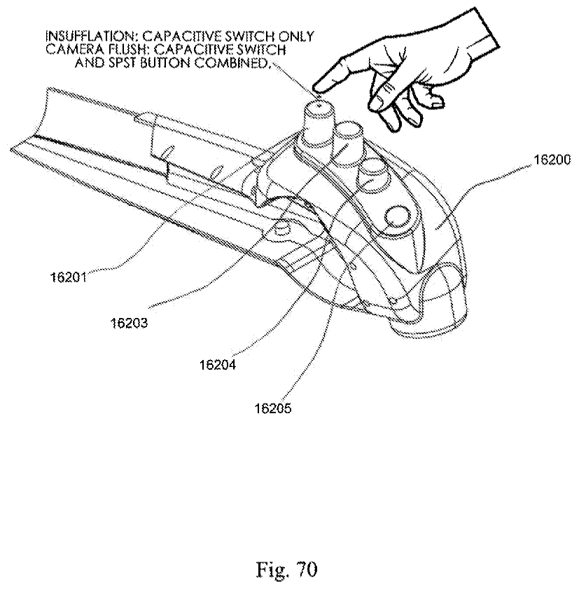

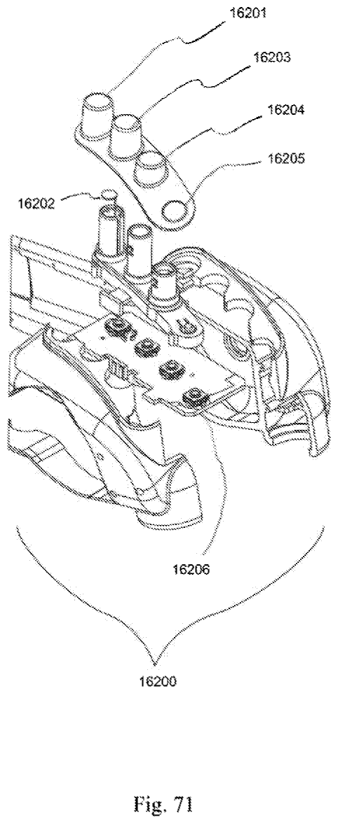

Referring to FIGS. 68-71, the articulation controls and an electronics module that incorporates control switches and navigation switches are mounted to the housing of the reusable hand-piece 16200, and the articulation wire actuating assembly and connector of the single-use shaft assembly 16400 are mounted to the housing of the single-use shaft assembly. Accordingly, separation of the housing of the reusable hand-piece from the housing of the single-use shaft assembly separates the articulation controls from the articulation wire actuating assembly and the electronic control switches and navigation switches from the connector of the single-use shaft assembly.

Some embodiments may include an electronics module that includes a plurality of switches mounted to the housing of the reusable hand-piece that is in electrical communication with the wire harness associated with the single-use shaft assembly via an electrical connector. The plurality of switches mounted to the housing of the reusable hand-piece that is in electrical communication with the wire harness associated with the single-use shaft assembly via an electrical connector. The electronics module of this embodiment may further incorporate a microprocessor to minimize the number of connectors required to communicate the specific switch or combination of switches that are depressed by the user.

In another embodiment of the endoscope, the reusable hand-piece incorporates an electronics module, switches, and electrical connector while the single-use shaft assembly incorporates an optical sensor, optical sensor module, and electrical connector; wherein, a set of conductors transmits both optical and control data back to a console from the assembled endoscope.

Advantageously, the incorporation of an electronics module and switches facilitates transmission of control data while simplifying the components associated with the separable single-use shaft.

The reusable hand-piece can include a circuit board (electronics module) arranged to control at least a portion of support equipment (e.g., one or more pumps and/or valves in support equipment). One or more switches of the re-usable hand-piece can be associated with the electronics module to control the support equipment.

Advantageously, such an arrangement can eliminate the need for valves for the irrigation and aspiration lumens to be physically associated with the reusable hand-piece and/or the separable distal shaft assembly.

The switches associated with the reusable hand-piece control board/circuit board 1206 (electronics module) incorporates two electrical switches actuated by discreet buttons 16204, 16205 for programmable functions at the top of the reusable hand-piece. The 2^{nd} switch from the bottom is actuated by a discreet button 16203 for activation of a remote suction valve 16101. The bottom button 16201 incorporates two electrical switches. The first switch 16202 associated with the bottom button is a capacitive switch that initiates the insufflation function via remote valve 16102 operation when the operator places a finger on the exterior molded gasket covering the button. Vent hole 16103 allows for insufflation gas to pass out of the endoscope assembly. The second switch associated with the bottom button is a discreet switch mounted on a control board that actuates that initiates the camera flush function via remote valve operation when the operator fully depresses the bottom button.

Advantageously, the use of remote valves associated with the single-use shaft cartridge eliminates the cleaning requirements and potential contamination associated with the mechanical valves associated with the state of the art for reusable scopes.

Advantageously, the capacitive switch associated with the bottom button provides the same user experience associated with the current state of the art reusable endoscopes with both simply requiring a finger placed on the top of the button to initiate insufflation with the current state of the art reusable endoscope simply having a vent hole located on top of the valve button. However, the capacitive insufflation switch eliminates the cleaning requirements and potential contamination associated with the mechanical valves associated with the state of the art for reusable scopes.

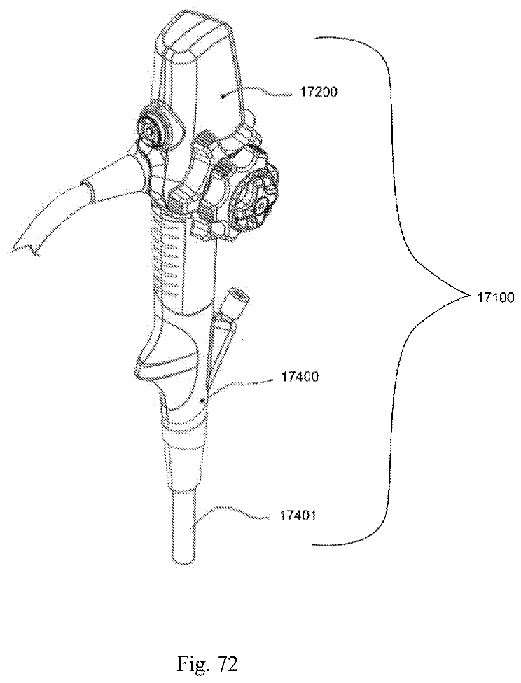

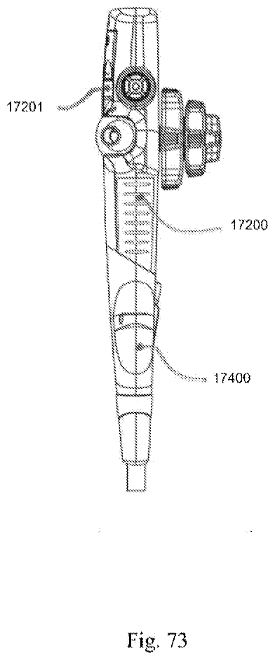

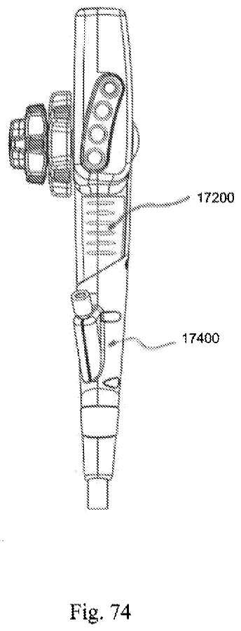

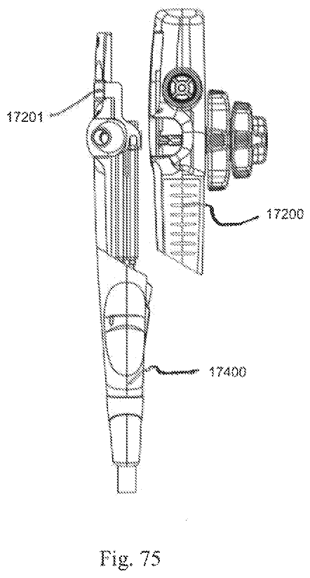

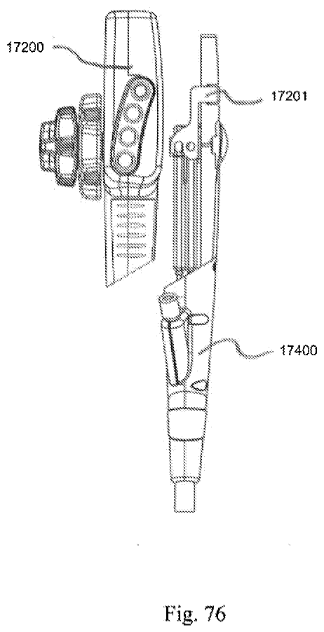

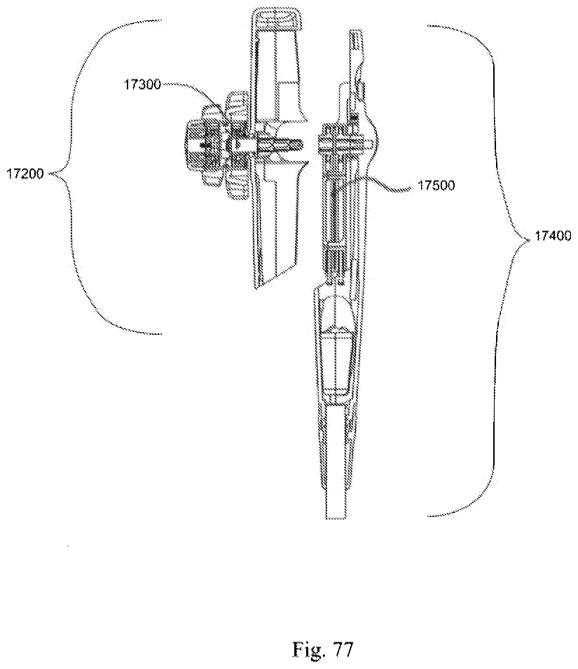

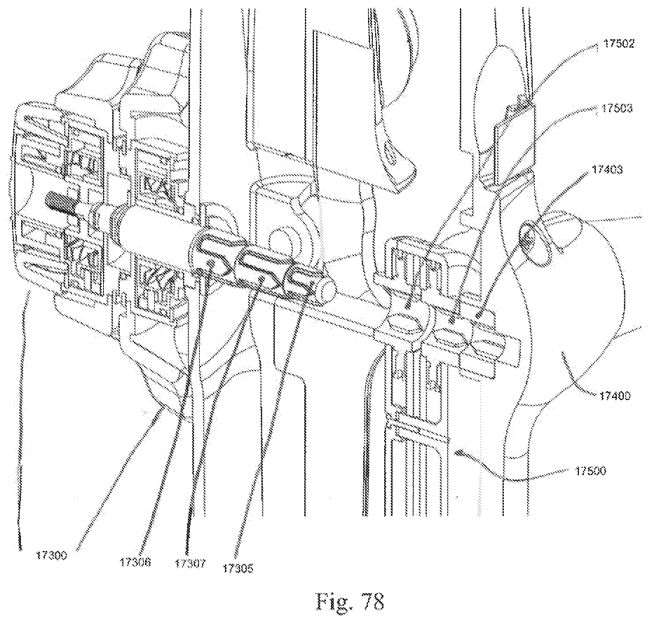

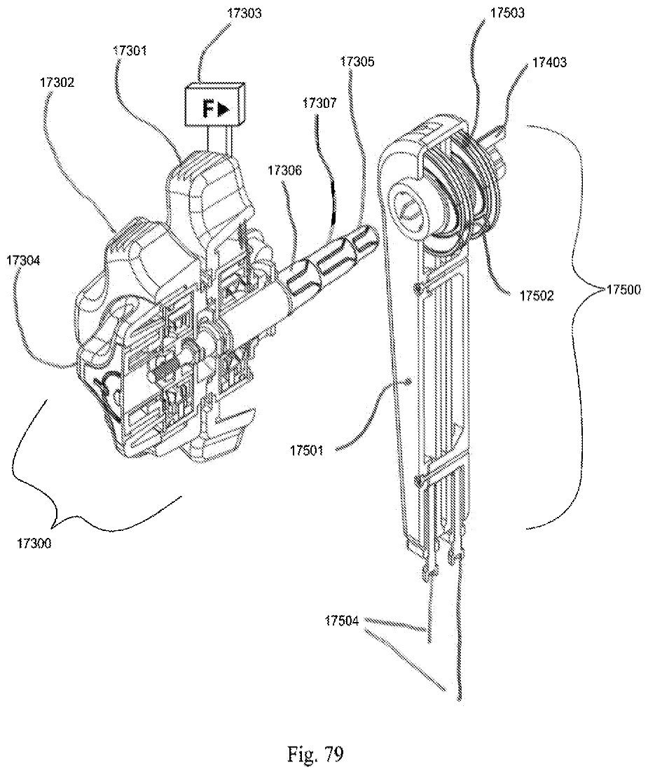

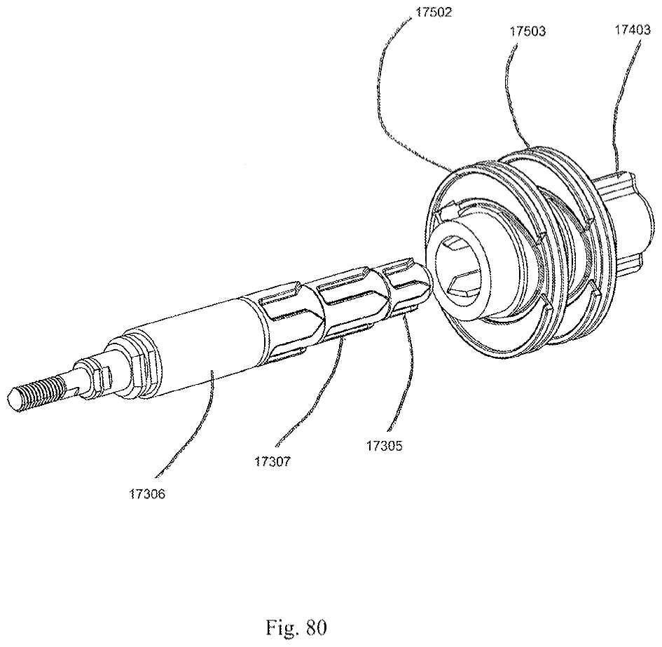

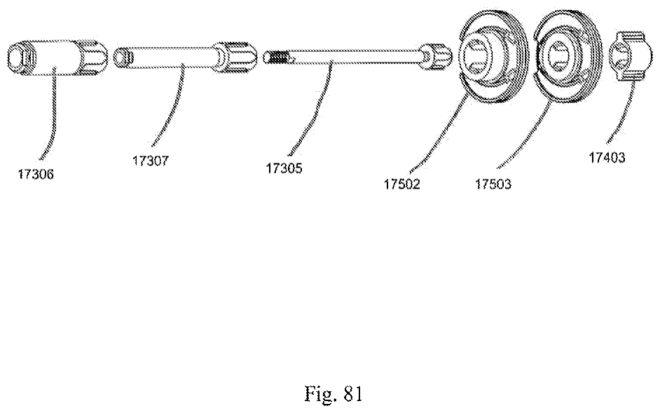

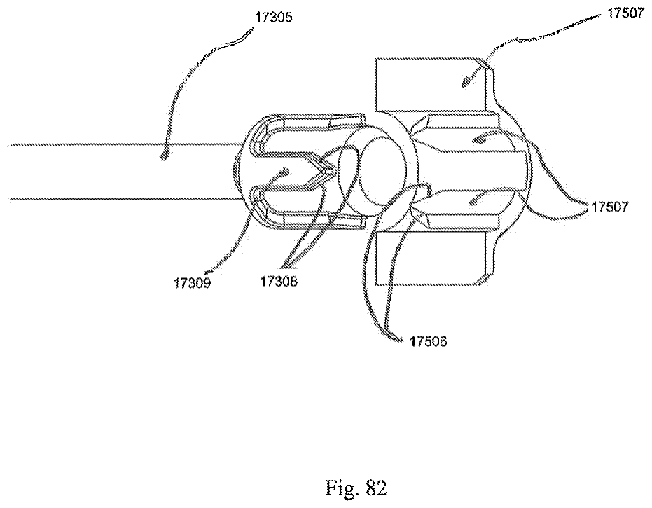

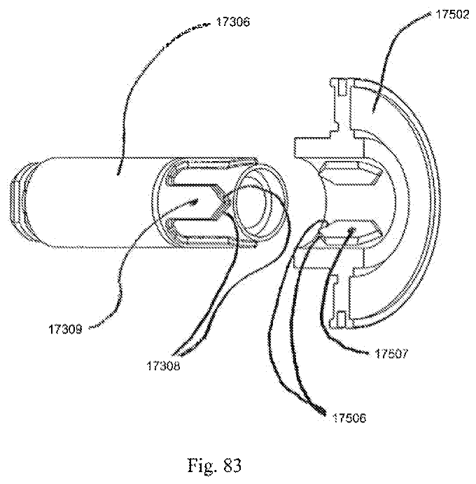

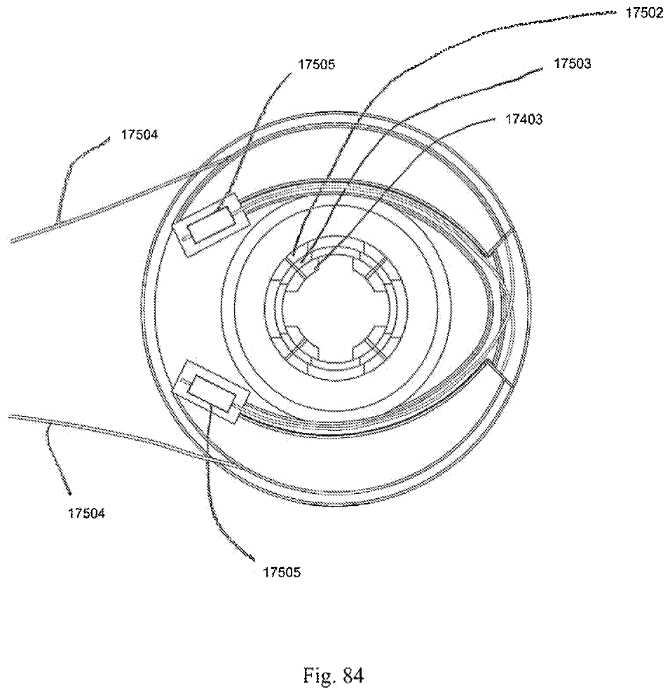

Referring to FIGS. 72-84, the endoscope assembly 17100 can include a coupler that holds the reusable hand-piece 17200 and the single-use shaft assembly 17400 together in the assembled configuration. The coupler can include a first portion on the reusable hand-piece and a second portion on the single-use shaft assembly, wherein the first and second portions of the coupler cooperate to hold the reusable hand-piece and the single-use shaft assembly together in the assembled configuration. The coupler may include a lever 17201. Discussion of lever 3001 is applicable to lever 17201 and is incorporated here.

The articulation controls of the reusable hand-piece can include an articulation knob and brake assembly 17300. The articulation knob and brake assembly of the reusable hand-piece can include an up-down articulation knob 17301, a left-right articulation knob 17302, an up-down articulation brake lever 17303 and a left-right articulation brake knob 17304. When the reusable hand-piece assembly is attached to the single-use shaft assembly, the articulation knob and brake assembly cooperates with the single-use shaft assembly to articulate a distal segment of the single-use shaft assembly and/or resist rotation of one or more articulation knobs relative to the endoscope assembly. For example, as discussed in more detail elsewhere herein, an up-down articulation shaft 17306 and a left-right articulation shaft 17307 and/or left-right articulation brake shaft 17305 of the articulation knob and brake assembly may engage the pulley assembly 17500 and/or housing of the single-use shaft assembly. The articulation knobs can be arranged to receive articulation input from the user (e.g., in the form of rotational movement). The articulation knobs can communication with pulley assembly 17500 of the separable single-use shaft to actuate control wires of the single-use shaft assembly. The user controlled articulation knobs can include two knobs having and/or rigidly attached to concentric drive shafts.

In another example, the first articulation control knob may control the left/right motion of the endoscope and the first articulation brake incorporates a left/right articulation to brake shaft concentrically located within the first articulation shaft that provides a fixed reference relative to the single-use shaft housing by engaging a socket 17403 of the single-use shaft housing. The first articulation shaft is concentrically aligned to the socket with a series of torque transmission bosses alternatingly engaged between the second shaft and the second pulley.

The concentric drive shafts each have a pulley engaging portion. This portion may have a non-circular cross-sectional geometric shape such that it is capable of transmitting torque. This geometric shape can include, but is not limited to, oval, spline, square, or star, just to name a few non-limiting examples.

Advantageously, the concentric shaft drive configuration permits attachment of the single-use shaft assembly to the reusable hand-piece without the need to orient the articulation knobs or the distal articulating tip to obtain a neutral reference position. Regardless of the orientation of the single-use shaft assembly (coiled or straight) at the time of attachment, the articulation knobs will freely rotate while the distal shaft 17401 (insertion tube) is manipulated prior to use by the clinician.

The single-use shaft assembly can include a one or more pulleys contained within a rigid housing 17501. For example, the single-use shaft assembly may have a proximal rigid housing having a surface that supports and positions the pulleys of the single-use shaft assembly to receive the pulleys engaging portions of the drive shafts.

Each pulley contained within the proximal rigid housing associated with the separable single-use shaft possess a pair of opposing articulation wires 17504. Each articulation wire can be rigidly fixed to the pulley/cam and on the other end to the distal tip of the articulating section of the shaft. Rotation of the pulley/cam, such as by means of a user input torque applied to the corresponding knob, results in a tensile force applied to the articulating distal section.

A preferred embodiment utilizes two pulleys 17502 and 17503, each associated with a pair of articulation wires. Each pair of articulation wires provides means for articulating the distal shaft section in a single plane, for example up-down and left-right. Two pairs of articulation wires provide distal shaft articulation in two different plans that are perpendicular to one another, with a longitudinal axis of the proximal shaft portion passing through the intersection of the planes. The articulation pull wires can be attached to the pulleys by articulation pull wire adhesive and/or an articulation pull wire crimp tube 17505.

The mating features of the concentric drive shafts associated with the reusable hand-piece and the mating features of the pulleys associated with the separable, single-use shaft assembly are suitable for transmitting force in the form of torque applied to the articulation knobs to tensile force in the articulation wire(s) and corresponding movement of the distal shaft articulating section.

A portion of the drive shafts, such as the pulley engaging portions, may be tapered along a length of the drive shafts. For example, the drive shaft may have a portion taper from small to large in a direction towards the articulation knob. Advantageously, such an arrangement can aid in mating the drive shafts with the pinions of the single-use shaft assembly when the reusable hand-piece is coupled to the single-use shaft assembly.

The reusable hand-piece can include a brake that applies an adjustable level of rotational resistance to the articulation control (e.g., articulation control knobs). A separate brake mechanism exists for each articulation control knob and its associated pair of articulation wires for applications where two plane articulation is required. Furthermore, the brake mechanism(s) have been consolidated within the envelope defined by the knobs, thereby creating space within the reusable hand-piece housing for the single-use shaft assembly. Furthermore, the brake mechanism(s) do not utilize a fixed method for applying force against the friction surfaces and/or brake material, such as rotation of a threaded shaft.

Advantageously, the reusable hand-piece of the endoscope assembly can remain entirely outside of the body of a patient during an endoscopic procedure while the single-use shaft assembly has a portion positioned within the body of the patient during the endoscopic procedure. Accordingly, the reusable hand-piece and single-use shaft assembly can be separated from one another after the procedure and the single-use hand-piece discarded (or reprocessed). As there are no fluid lumens of the reusable hand-piece that must be cleaned and sterilized, the cleaning (aka “reprocessing”) effort between procedures is dramatically reduced. Additionally, as no portion of the reusable hand-piece, which is used for multiple patients, is inserted into the patient, the risk of infectious disease transmission can be dramatically reduced.

Single-use shaft assemblies can be arranged and/or provided in a variety of configurations to support upper and lower endoscopies. For example, single-use shaft assemblies may be arranged for colonoscope, gastroscope, sigmoidoscope, and/or duodenoscope procedures, just to name a few non-limiting examples. Additionally or alternatively, single-use shaft assemblies can be provided in various specialty configurations, e.g. pediatric insertion tube diameters. Advantageously, the ability to use the same reusable hand-piece for a variety of single-use shaft assemblies and/or procedures can substantially reduce capital investments by clinicians, clinician groups, and/or medical centers by eliminating the need to stock a plurality of dedicated scopes for each type of procedure, e.g. colonoscope, gastroscope, sigmoidoscope, duodenoscope, etc.

Methods of assembling an endoscope assembly, disassembling an endoscope assembly, and/or using an endoscope assembly are envisioned. Such methods can comprise connecting a housing of a reusable hand-piece to a housing of a single-use shaft assembly, wherein said reusable hand-piece has articulation controls and an electronics module and said single-use shaft assembly has an articulation wire actuating assembly and a connector; and wherein said connecting connects the articulation controls to the articulation wire actuating assembly and the control module to the connector of the single-use shaft assembly. Additionally, or alternatively, methods can comprise separating the housing of the reusable hand-piece from the housing of the single-use shaft assembly so as to separate the articulation controls from the articulation wire actuating assembly and the control module from the connector of the single-use shaft assembly. The methods can include providing a portion, or all, of any endoscope assembly described herein.

Advantageously, the systems, assemblies, devices, and methods disclosed herein can increase the quantity of endoscopy procedures that can be performed by a clinician and/or facility in a day by reducing and/or eliminating the time delays associated with existing reusable scopes that must undergo extensive reprocessing procedures (i.e., cleaning) between use. By using a single-use shaft, it is no longer necessary for the clinician and/or facility to reprocess (i.e., clean) the shaft and lumens of the shaft. Now the clinician and/or facility may simply wipe down the reusable hand-piece and connect a new, sterilized, single-use shaft assembly to the reusable hand-piece to prepare the endoscope assembly for another procedure.

Advantageously, the systems, assemblies, devices, and methods disclosed herein can allow a clinician to perform multiple and/or various procedures even without dedicated, reusable scopes and associated reprocessing equipment, supplies, and clean water. This can be particularly advantageous in battlefield settings or remote clinics with limited resources. In these cases, the reprocessing equipment, reprocessing supplies, trained reprocessing personnel, and reprocessing laboratory setting may not be available. Advantageously, the endoscopes disclosed herein can be prepared for a new procedure by simply using a wipe and antiseptic solution to clean the exterior surfaces of the reusable hand-piece and connecting a new single-use shaft assembly.

Advantageously, the reusable hand-piece can provide user-familiar-features using higher precision reliable components associated with the articulation knobs and the brake.

Advantageously, incorporating the articulation handles and clutch into the reusable hand-piece reduces the components of the separable single-use shaft assembly thus resulting in a lower cost single-use portion of the endoscope.

The single-use shaft assemblies disclosed herein can be intended for one-time-use. Advantageously, a single-use medical device can reduce transmission of infectious diseases.

Advantageously, the preferred embodiment illustrated incorporates a series of three or more torque transmission bosses 17309 and 17506 with symmetric alignment ramps 17308 and 17507 that facilitate alignment of the articulation shafts with the articulation pulleys with the single-use shaft assembly is attached to the reusable hand-piece. These symmetric alignment ramps may be generated by a helical sweeping cut or by a linear angle cut.

Applicant has also observed that expertise associated with the assessment of particular diseases and the performance of novel therapeutic procedures has become increasingly concentrated at clinical research institutions or larger healthcare facilities. Advantageously, the endoscopes disclosed herein can facilitate the dissemination of patient and image data.

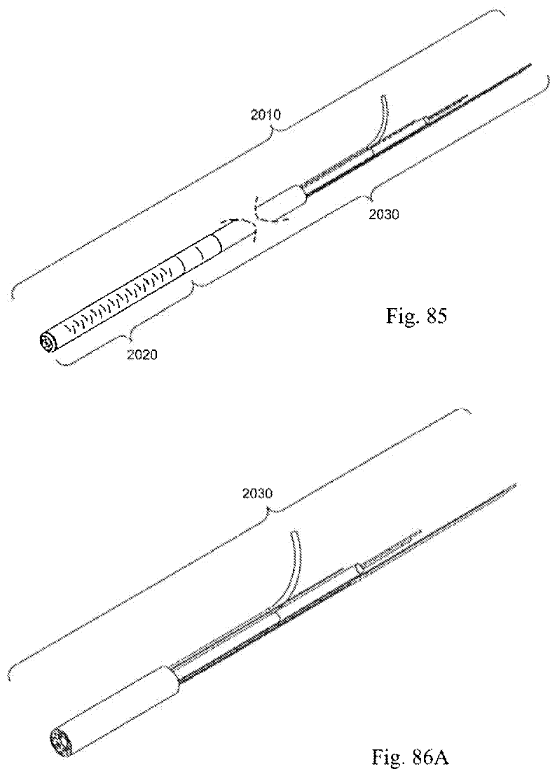

FIG. 85 illustrates a disposable flexible endoscope shaft assembly 2010 having a distal portion comprising an articulating section assembly 2020 and a proximal portion comprising an insertion tube assembly 2030.

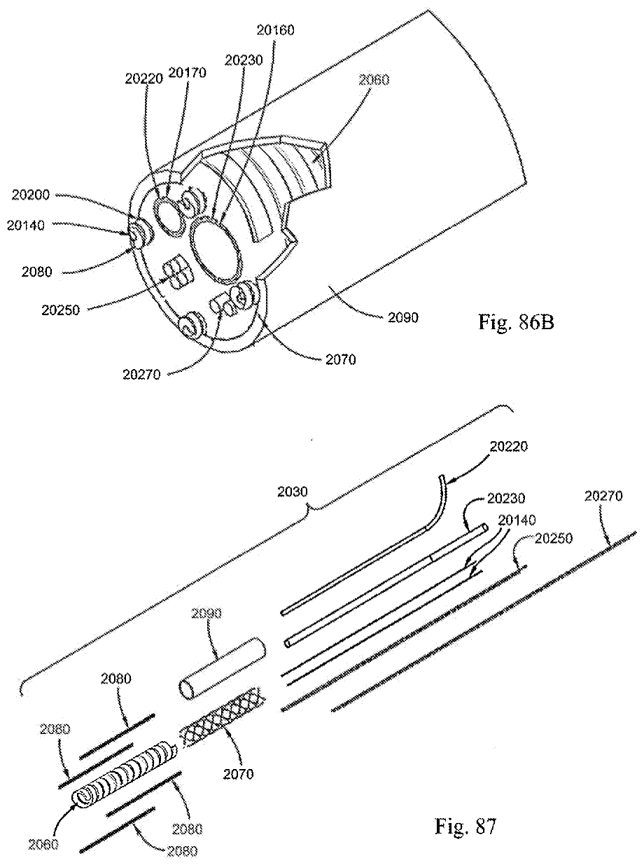

Turning now to FIGS. 86A, 86B and 87, the insertion tube assembly 2030 includes an outer coil 2060. An articulation wire 20140 and a compression coil 2080 extend along a length of outer coil 2060 through its interior.

A braided sleeve 2070 surrounds the outer coil 2060 and is positioned between the outer coil and an outer sheath 2090. The outer sheath 2090 can be applied over the braided sleeve 2070 and outer coil 2060 as a reflowed tube (e.g., thermal lamination) or by extrusion.

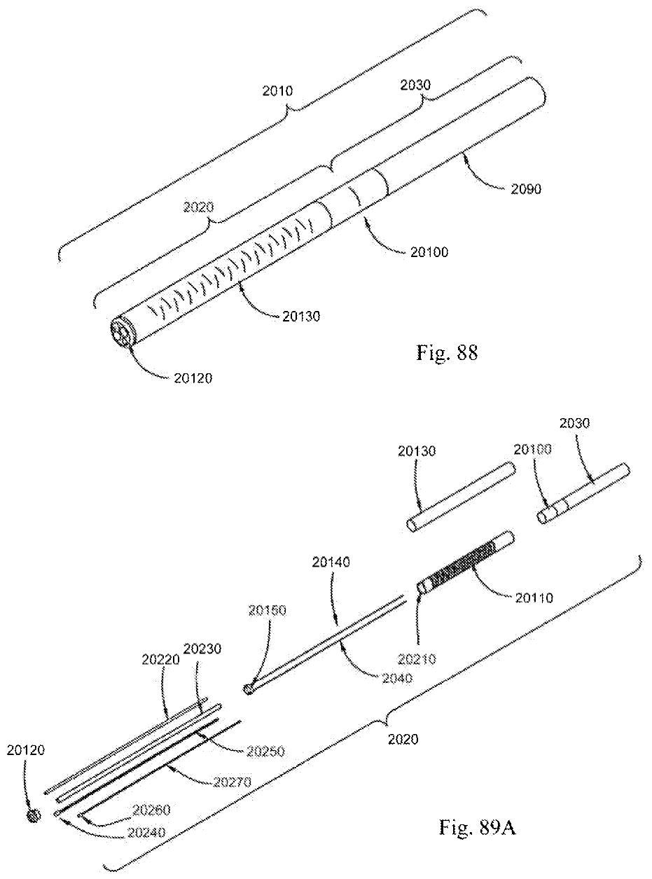

FIG. 88 illustrates a cap 20120 at the distal end of the articulating section 2020 and an articulating section outer sheath 20130. The articulating section assembly 2020 is connected to the insertion tube assembly 2030 with a transition tube 20100. The transition tube 20100 may be mechanically secured to both the articulating section assembly 2020 and the insertion tube assembly 2030 through a process such as swaging.

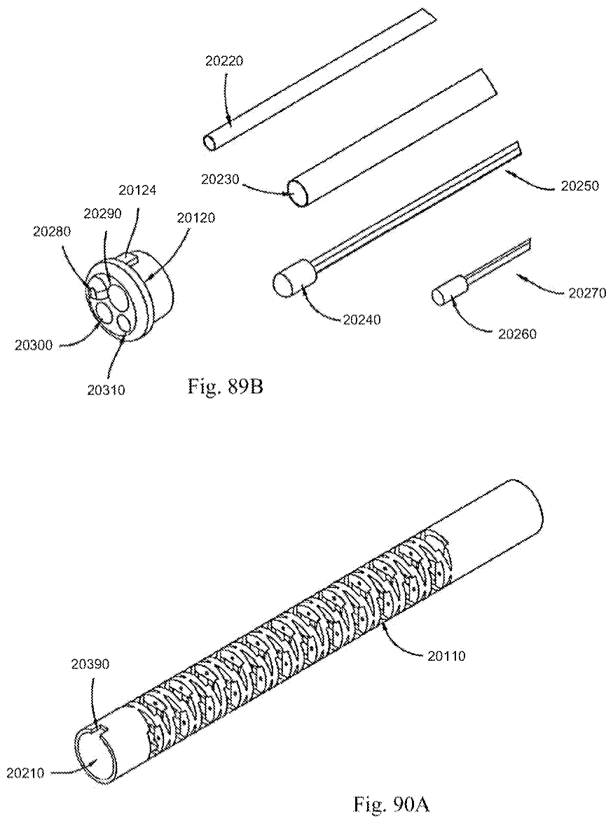

FIGS. 89A and 89B depict the articulating section assembly 2020. The articulating section assembly 2020 comprises an articulation wire and termination ring assembly 2040 having a termination ring 20150 positioned at a distal end of articulation wires 20140, a unitary articulating section 20110, an articulating section outer sheath 20130, cap 20120, an instrument tube 20230, an air/water tube 20220, a camera 20240 and camera wiring harness 20250, a light emitter 20260 (e.g., an LED) and light emitter wiring harness 20270. When disposable flexible endoscope shaft assembly 2010 is assembled, air/water tube 20220 extends through a lumen 20210 defined by the articulating section 20110, instrument tube 20230 extends through lumen 20210, camera wiring harness 20242 extends through lumen 20210, and light emitter wire harness 20270 extends through lumen 20210.

Cap 20120 defines an air/water nozzle 20280, an instrument tube outlet 20290, a camera outlet 20300, and a light emitter outlet 20310. Cap 20120 includes a cap alignment tab 20124 arranged to engage a cap alignment notch of an articulating section (e.g., unitary articulating section 20110).

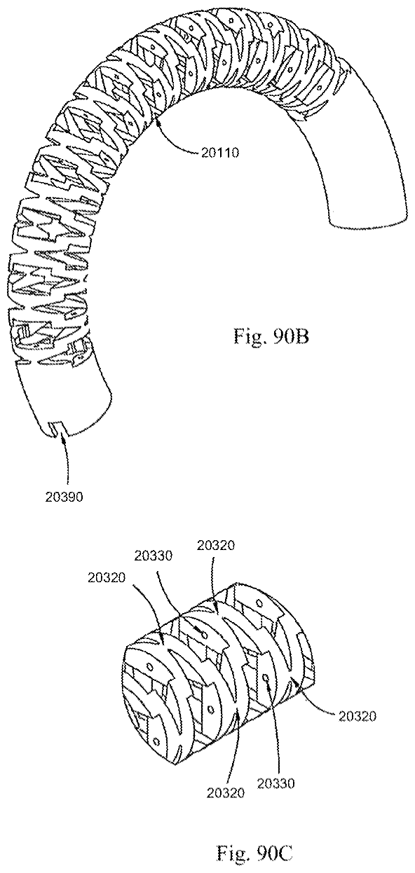

FIGS. 90A-90C depict the unitary articulating section 20110 comprising a plurality of living hinges 20320 being alternatingly located in two perpendicular planes when unitary articulating section is in a straight configuration. Each deformable living hinge element 20320 provides means for rotation around a pivot axis in a single plane. An articulation wire lumen 20330 traverses each living hinge element 20320 and is arranged to receive an articulation wire 20140.

The lumen 20210 located within the unitary articulating section 20110 can receive an air/water tube, an instrument tube, and/or wiring 20210. A cap alignment notch 20390 is located at the distal end of the unitary articulating section 20110 and arranged to receive the cap alignment tab 20124.

FIG. 90B illustrates the unitary articulating section 20110 in a deformed configuration consistent with an articulation of 180° for retrograde viewing using the distally mounted camera 20240.

FIG. 91A illustrates the proximal end of the unitary articulating section 20110, and FIG. 91B illustrates the distal end of the unitary articulating section 20110. As seen in FIG. 91B, a distally facing surface 20334 extends inwardly from the inner surface 20336 of the unitary articulating section 20110. Distally facing surface 20334 is arranged to contact termination ring 20150 and transfer tensile force from the articulation wire and termination ring assembly 2040 to the unitary articulating section 20110.

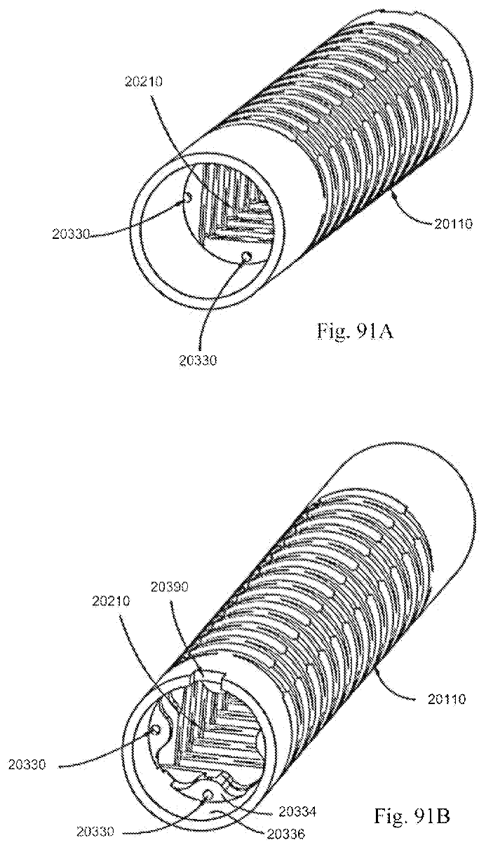

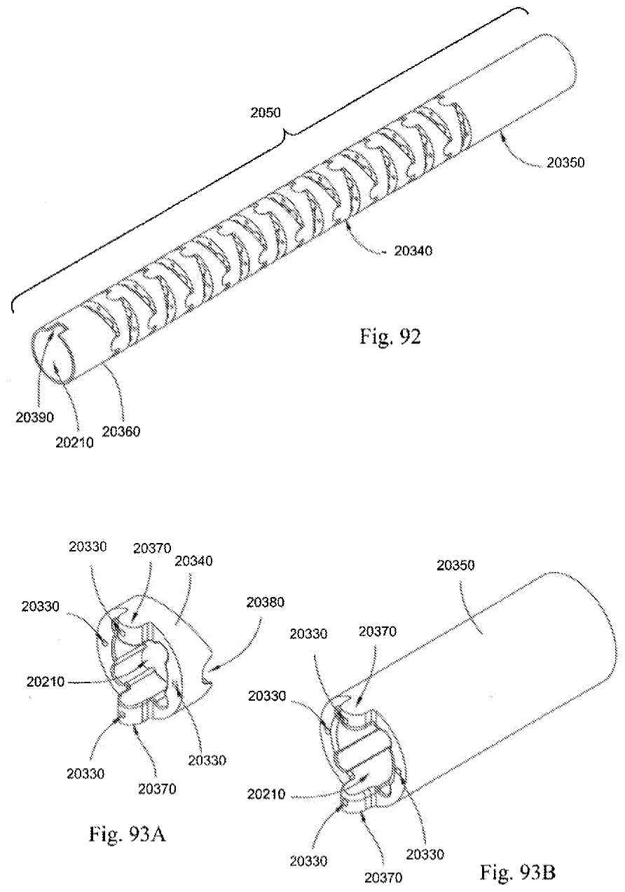

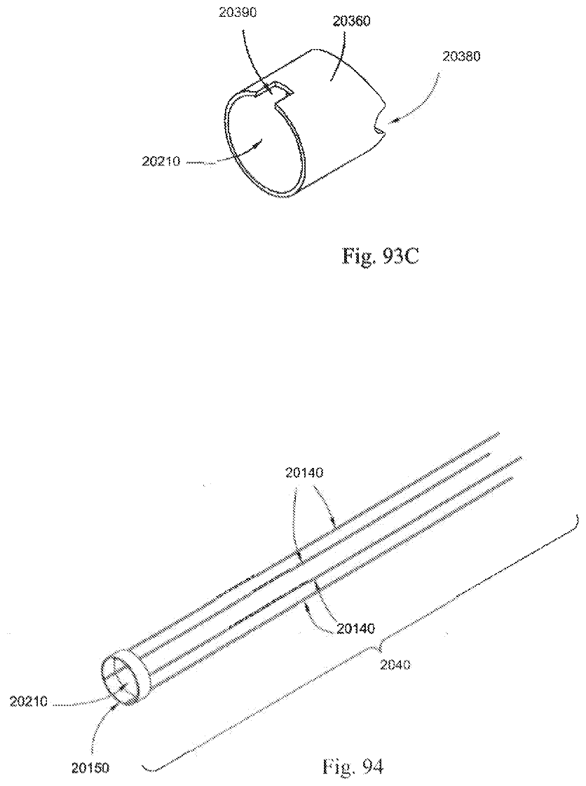

FIGS. 92-93C illustrate an articulating link assembly 2050 comprising a proximal articulating link 20350, an intermediate articulating link 20340, and a distal articulating link 20360. The proximal articulating link 20350, intermediate articulating link 20340, and distal articulating link 20360 each define a lumen 20210 arranged to receive an air/water tube, an instrument tube, and/or wiring.

The proximal articulating link 20350 includes pivot tabs 20370 located in a first (e.g., vertical) plane. The intermediate articulating link 20340 comprises articulation pull wire lumens 20330, pivot tabs 20370 located in a first plane, and pivot sockets 20380 located in a second plane. The distal articulating link 20360 includes a cap alignment notch 20390 to control alignment of the camera 20240 relative to each of the four articulation pull wires 20140 and two pivot sockets 20380 located in the first or second plane. When assembled, the pivot tabs 20370 are received within and pivotable relative to the pivot sockets 20380.

FIG. 94 illustrates the articulation pull wire and termination ring assembly 2040 comprising four articulation pull wires 20140 and an articulation pull wire termination ring 20150. The interior of the articulation pull wire termination ring 20150 defines lumen 20210 for passage of air/water tube, instrument tube, and wiring.

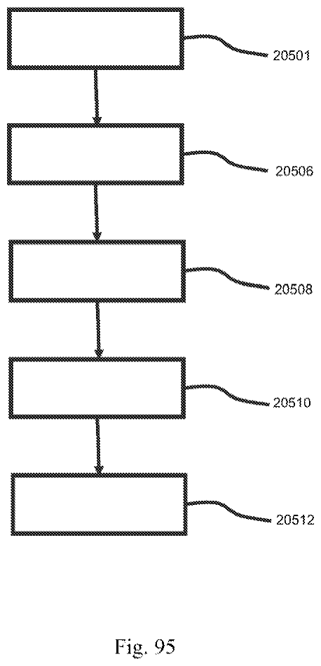

Turning now to FIG. 95, a process for manufacturing insertion tube assemblies disclosed herein is described. The insertion tube assemblies can be manufactured using a continuous manufacturing process (e.g.., reel-to-reel). The process can begin in stage 20501 with a continuous coiling process wherein the coil is manufactured in a continuous way (e.g., a continuous wire coiler).

In stage 20506, the braided sleeve can be applied around the outer coil. This can also occur during a continuous manufacturing process.

In stage 20508, the outer sheath can be applied. This, again, can occur during a continuous manufacturing process. For example, the outer coil and braided sleeve assembly can pass through one or more extrusion heads that extrude the outer sheath around the assembly. Such a process can create a smooth outer sheath that is integrally bonded to the outer coil and/or braided sleeve. The outer sheath may have a varying durometer along its length such that some areas (e.g., lengths) of the outer sheath have a greater durometer than other areas. This may be accomplished by extruding resins of different durometers (e.g., different resins) through separate extrusion heads of the one or more extrusion heads or by extruding resins of different durometers through at least one extrusion head of the one or more extrusion heads.In stage 20510, the desired length of insertion tube assembly, or a portion thereof, can be cut to length. This can occur immediately after the outer sheath extrusion process or, for example, from a reel of finished goods. After cutting the assembly to length, articulation wires and/or compression coils may be inserted into the lumen of the outer coil. The process concludes in stage 20512.

As will be appreciated by those skilled in the art, this continuous technique for fabricating the insertion tube avoids labor and time intensive batch processes currently used to form discrete sections of endoscope shafts. Applicant believes this can reduce the costs of manufacturing endoscope shaft assemblies and/or increase production speed.

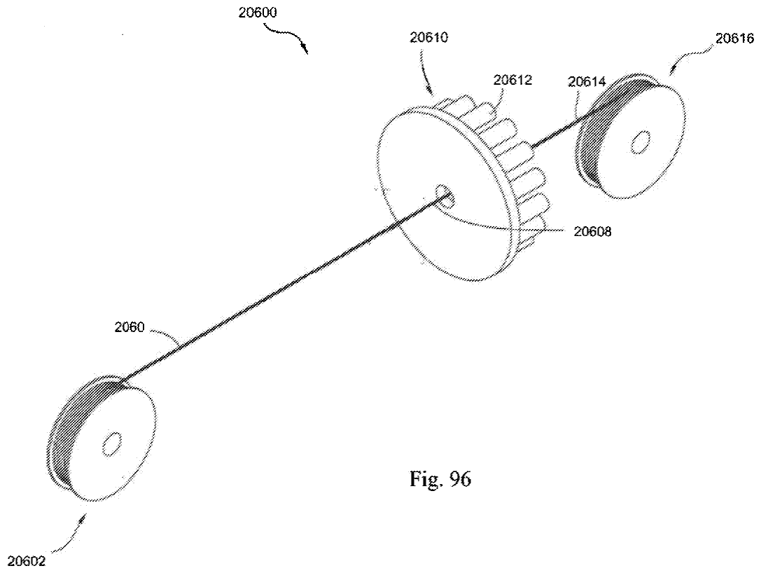

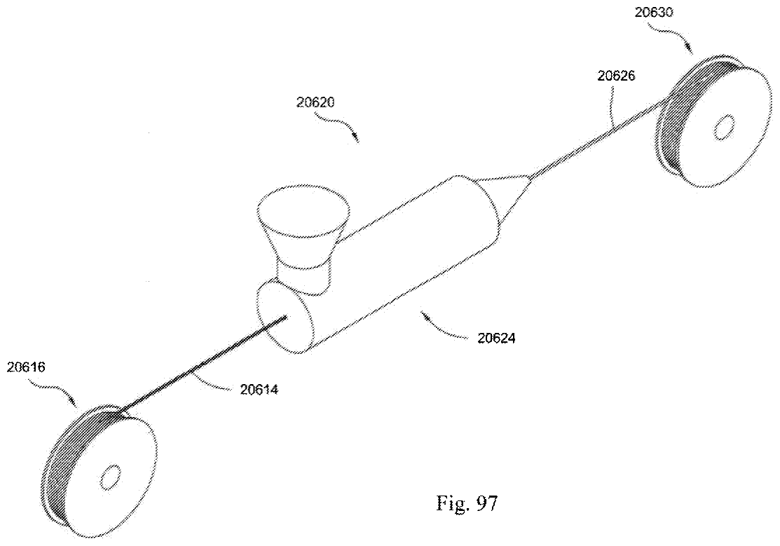

FIGS. 96 and 97 illustrate a manufacturing arrangement suitable for practicing the process described above. In FIG. 96, a first portion 20600 of the manufacturing arrangement is shown. First portion 20600 includes an outer coil 2060 extending from an outer coil reel 20602 to fixture 20610. Fixture 20610 is arranged to form a braided sleeve around the outer coil as the outer coil and compression coil continually advances through fixture 20610. For example, fixture 20610 may include a braiding machine having a plurality of bobbins 20612 that weave relative to one another to form a braided (e.g., woven) sleeve 20608 around the outer coil. The outer coil and braided sleeve assembly 20614 can then extend to a reel 20616.

FIG. 97 illustrates a second portion 20620 of the manufacturing arrangement. In second portion 20620, the outer coil and braided sleeve assembly 20614 extends from reel 20616 to extrusion mold 20624. Extrusion mold 20624 is arranged to continuously extrude an outer sheath around the assembly 20614 as the assembly is advanced therethrough. The outer sheath assembly 20626 extends from the extrusion mold 20624 to a finished goods reel 20630.

While one manufacturing arrangement has been illustrated and described, the present disclosure is not limited to such. For example, reel 20616 may be omitted and the outer coil and braided sleeve assembly 20614 may extend directly from fixture 20610 to the extrusion mold 20624.

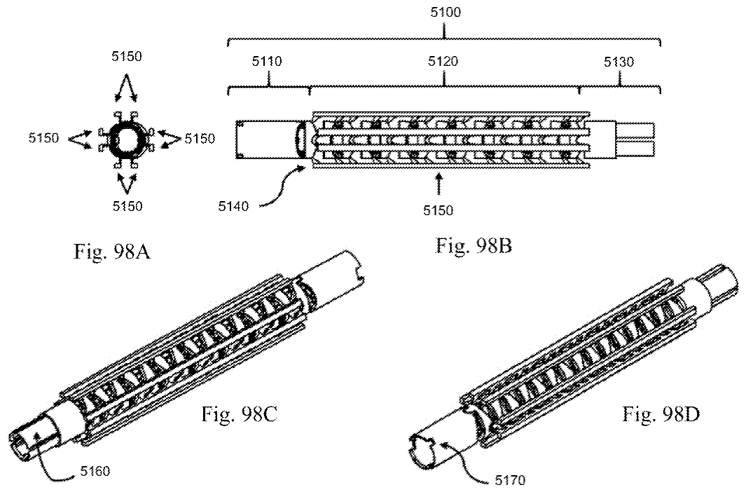

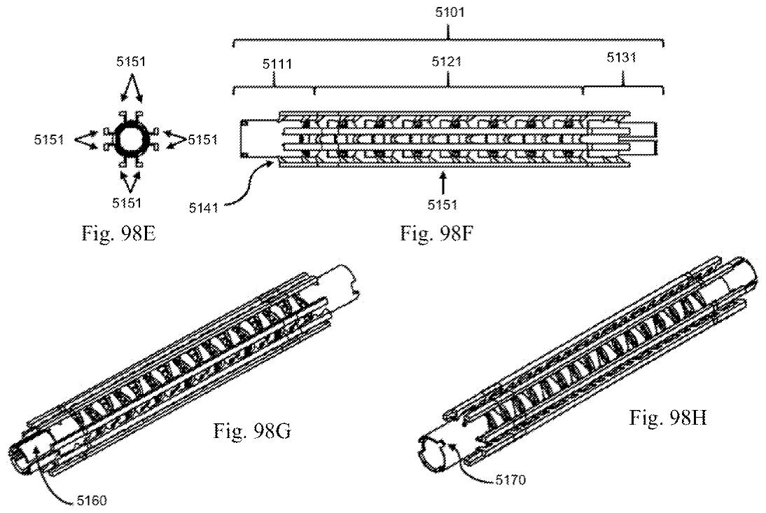

FIGS. 98A and 98B depict embodiments of an articulating joint assembly 5100 and 5101. The articulating joint assembly 5100 and 5101 may compromise a proximal link 5130, an articulating section assembly 5120, a distal link 5110, and one or more breakaway struts 5150 and 5151. Each breakaway strut 5150 may be attached to individual articulating links of an articulating section assembly 5120 with one or more individual connection tabs 5140 for alignment of the individual articulating links.

Additionally, one or more breakaway struts 5150 and 5151 may be attached to a proximal link 5130 or 5131 , a distal link 5110 or 5111, or both by one or more individual connection tabs 5140 or 5141 for the alignment of a proximal link 5130 and a distal link 5110 with the individual articulating links 5120 or 5121.

The proximal link 5130 may comprise one or more insertion tube engagement fingers 5160. The distal link 5110 may comprise one or more distal link keyed features 5170 for specific connection with an interfacing distal cap.

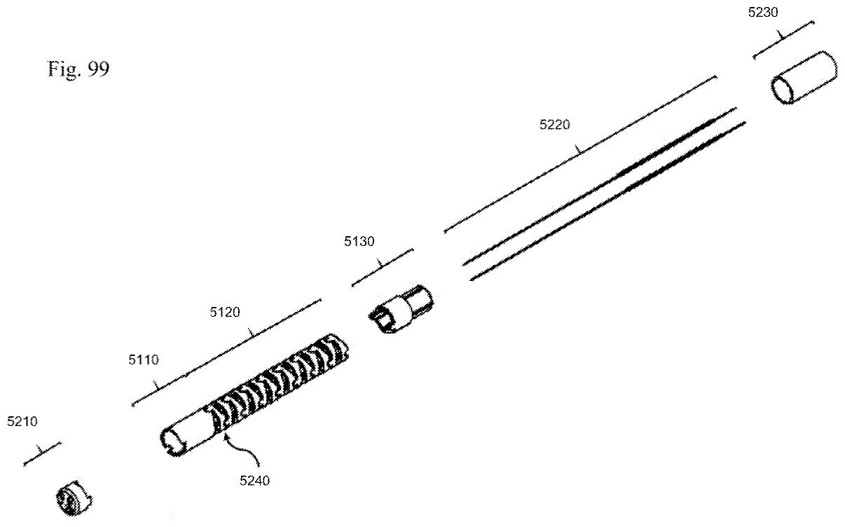

FIG. 99 depicts an exploded view of an articulating joint assembly 5100 and 5101 in association with a distal cap 5210, articulation steering wires 5220, and a proximal insertion tube 5230. The articulating joint assembly 5100 and 5101 includes individual articulating links 5240 and may control the alignment of the distal link 5110, distal cap 5210, or both by selective tensioning of one or more articulation steering wires 5220 of the articulating joint.

One or more breakaway struts 5150 and 5151 align the articulation joint assembly 5100 and 5101 to insert wires through articulation steering cable channels.

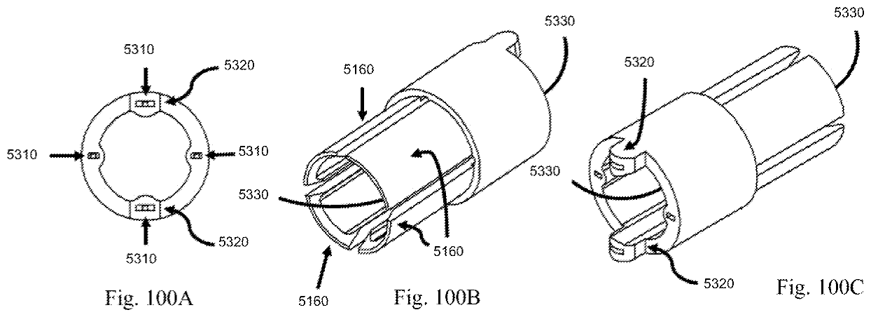

FIG. 100 depicts a proximal articulating link 5130 comprising pivot tabs 5320 located at the distal face, articulating steering cable channels 5310 traversing the length of the proximal articulating link, and insertion tube engagement fingers 5160 located on the proximal face.

A utility lumen 5330 traverses through the proximal articulating link 5130. The utility lumen 5330 provides space for instrument tubes, air tubes, water tubes, camera wire harness, LED wire harness, compression coils, medical tools, or any combination thereof.

Additionally, an embodiment may include a proximal articulating link 5130 comprising pivot sockets at the distal face rather than pivot tabs 5320.

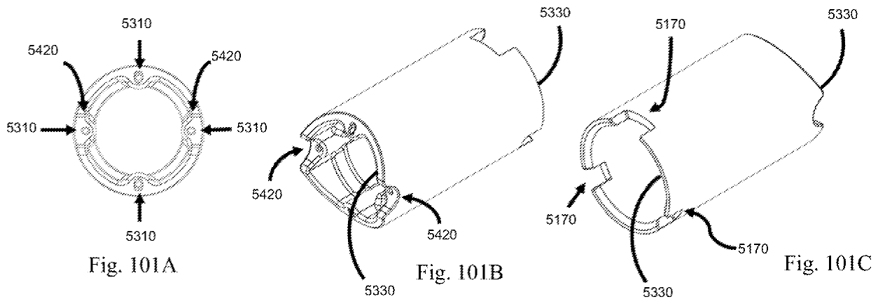

FIG. 101 depicts a distal articulating link 5110 comprising pivot sockets 5420 located at the proximal face, articulating steering cable channels 5310 traversing the length of the distal articulating link, and distal link keyed features 5170.

A utility lumen 5330 traverses through the distal articulating link 5110. The utility lumen 5330 provides space for instrument tubes, air tubes, water tubes, camera wire harness, LED wire harness, compression coils, medical tools, or any combination thereof.

Additionally, an embodiment may include a distal articulating link 5110 comprising pivot tabs at the proximal face rather than pivot sockets 5420.

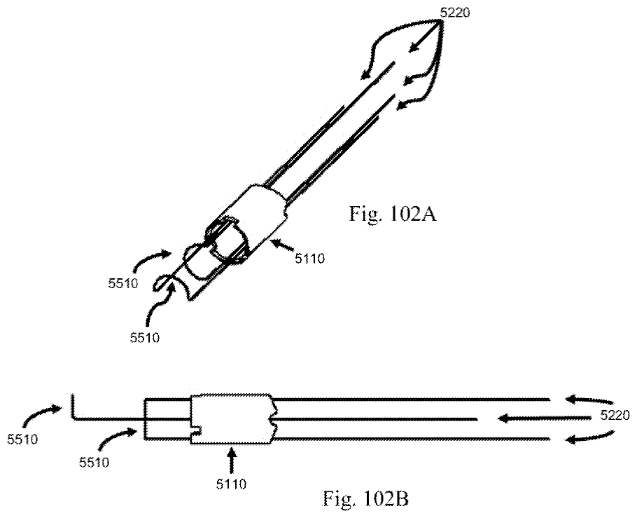

FIG. 102 depicts a distal articulating link 5110 assembly with articulation steering wires 5220 and wire termination 5510. Wire termination 5510 may include individual articulation wire termination at a termination ring. A wire termination 5510 embodiment may include a continuation of wire between articulation steering wires 5220 traversing adjacent articulating steering cable channels 5310. Additionally, wire termination 5510 embodiment may include a continuation of wire between articulation steering wires 5220 along the periphery of an internal circumference of the utility lumen 5330 to opposing articulating steering cable channels 5310. The wire termination 5510 may be recessed within the distal face or along the internal perimeter of the distal region of the distal link 5110.

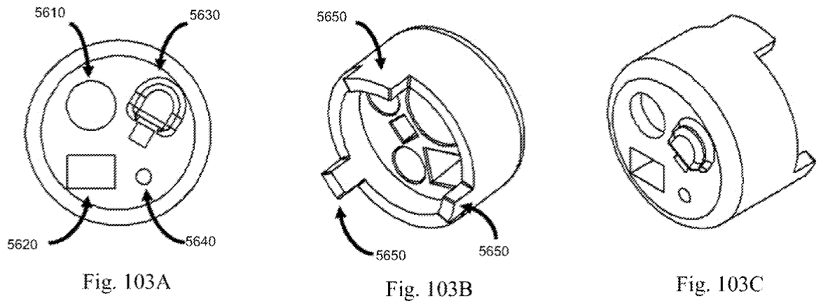

FIG. 103 depicts a perspective view of a distal end cap 5210. A distal end cap 5210 comprises a camera outlet 5610, a light emitter outlet 5620, an outlet for water, air, or both 5640, an instrument tube outlet, and cap keyed features 5650 that allow for the attachment to the distal link 5110 by mating with distal link keyed features 5170.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, “left”, “right”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

The following numbered clauses set out specific embodiments that may be useful in understanding the present invention:
1. An endoscope, comprising:
   a reusable hand-piece assembly and a single-use shaft assembly;
   the reusable hand-piece assembly selectively attachable to and detachable from the single-use shaft assembly;
   the reusable hand-piece assembly including a housing, a first articulation control knob, a second articulation control knob, a first articulation brake, and a second articulation brake; and
   the single-use shaft assembly including a housing and an endoscope shaft;
   wherein the first articulation brake is configurable from a disengaged position that permits rotation of the first articulation control knob relative to the housing of the reusable hand-piece to an engaged position that resists rotation of the first articulation control knob relative to the housing of the reusable hand-piece; and
   wherein the second articulation brake is configurable from a disengaged position that permits rotation of the second articulation control knob relative to the housing of the single-use shaft assembly to an engaged position that resists rotation of the second articulation control knob relative to the housing of the single-use shaft assembly when the reusable hand-piece assembly is attached to the single-use shaft assembly.
2. The endoscope of clause 1, wherein the second articulation brake includes a brake articulation shaft extending through the first articulation control knob.
3. The endoscope of clause 2, wherein when the reusable hand-piece assembly is engaged to the single-use assembly an end of the brake articulation shaft engages the housing of the single-use shaft assembly so as to rotationally fix the brake articulation shaft relative to the single-use shaft assembly.
4. The endoscope of clause 2 or 3, wherein the brake articulation shaft extends through a first articulation control shaft associated with the first articulation control knob; and
   wherein the first articulation control shaft rotates relative to the housing of the reusable hand-piece assembly upon rotation of the first articulation control knob relative to the housing of the reusable hand-piece assembly.
5. The endoscope of clause 4, wherein a second articulation control shaft associated with the second articulation control knob extends through the first articulation control shaft; and
   wherein the second articulation control shaft rotates relative to the housing of the reusable hand-piece assembly upon rotation of the second articulation control knob relative to the housing of the reusable hand-piece assembly.
6. The endoscope of clause 5, wherein the brake articulation shaft extends through the second articulation control shaft.
7. The endoscope of any one of clauses 4-6, wherein the first articulation brake includes an outer brake housing and a caliper;
   wherein the outer brake housing is associated with the first articulation control knob and first articulation control shaft such that rotation of the first articulation control knob rotates the outer brake housing and the first articulation control shaft;
   wherein the caliper is rotationally fixed relative to the reusable hand-piece; and
   wherein the caliper moves towards the outer brake housing when the first articulation brake is configured from the disengaged configuration to the engaged configuration.
8. The endoscope of clause 7, wherein the first articulation brake includes an inner brake housing positioned within the outer brake housing; and
   wherein the inner brake housing rotates relative to the caliper so as to translate the caliper towards the outer brake housing when the first articulation brake is configured from the disengaged configuration to the engaged configuration.
9. The endoscope of clause 8, wherein the inner brake housing is rotationally fixed to a brake control lever.
10. The endoscope of clause 8 or 9, wherein the outer brake housing, the inner brake housing, and the caliper are all positioned within the first articulation control knob.
11. The endoscope of any one of clauses 5-10, wherein the second articulation brake includes an outer brake housing and a caliper;
   wherein the outer brake housing is associated with the second articulation control knob and second articulation control shaft such that rotation of the second articulation control knob rotates the outer brake housing and the second articulation control shaft;
   wherein the caliper is rotationally fixed relative to the brake articulation shaft; and
   wherein the caliper moves towards the outer brake housing when the second articulation brake is configured from the disengaged configuration to the engaged configuration.
12. The endoscope of clause 11, wherein the second articulation brake includes an inner brake housing positioned within the outer brake housing; and
   wherein the inner brake housing rotates relative to the caliper so as to translate the caliper towards the outer brake housing when the second articulation brake is configured from the disengaged configuration to the engaged configuration.
13. The endoscope of clause 12, wherein the inner brake housing is rotationally fixed to a brake control knob.
14. The endoscope of clause 12 or 13, wherein the outer brake housing, the inner brake housing, and the caliper are all positioned within the second articulation control knob.
15. The endoscope of any preceding clause, wherein the first articulation control knob is positioned between the housing of the reusable hand-piece and the second articulation control knob.
16. An endoscope, comprising:
   a hand-piece assembly including a housing, a first articulation control knob, a second articulation control knob, a first articulation control shaft, a second articulation control shaft, a first articulation brake, and a second articulation brake;
   a first articulation control shaft associated with the first articulation control knob;
   wherein the first articulation control shaft rotates relative to the housing upon rotation of the first articulation control knob relative to the housing;
   wherein the second articulation control shaft rotates relative to the housing upon rotation of the second articulation control knob relative to the housing;
   wherein the first articulation brake is configurable from a disengaged position that permits rotation of the first articulation control shaft relative to the housing to an engaged position that resists rotation of the first articulation control shaft relative to the housing;
   wherein the second articulation brake is configurable from a disengaged position that permits rotation of the second articulation control shaft relative to the housing to an engaged position that resists rotation of the second articulation control shaft relative to the housing; and
   wherein the second articulation brake includes a brake articulation shaft extending through the second articulation control shaft.
17. The endoscope of clause 16, wherein the brake articulation shaft is rotationally fixed relative to the housing.
18. The endoscope of clause 16 or 17, wherein the second articulation control shaft extends through the first articulation control shaft.
19. The endoscope of any one of clauses 16-18, wherein the first articulation control shaft extends through a steering knob hub that is rotationally fixed relative to the housing.
20. The endoscope of clause 19, wherein the first articulation control knob is rotationally fixed relative to the steering knob hub when the first articulation brake is configured in the engaged position.
21. An endoscope, comprising:
   a hand-piece assembly including a housing, a first articulation control shaft, a second articulation control shaft, a first articulation brake, and a second articulation brake;
   wherein the first articulation control shaft has a portion positioned inside of the housing and a portion positioned outside of the housing;
   wherein the second articulation control shaft has a portion positioned inside of the housing and a portion positioned outside of the housing;
   wherein the first articulation brake is configurable from a disengaged position that permits rotation of the first articulation control shaft relative to the housing to an engaged position that resists rotation of the first articulation control shaft relative to the housing;
   wherein the second articulation brake is configurable from a disengaged position that permits rotation of the second articulation control shaft relative to the housing to an engaged position that resists rotation of the second articulation control shaft relative to the housing; and
   wherein the first articulation brake is positioned at least partially around the first articulation control shaft portion outside of the housing.
22. The endoscope of clause 21, wherein the second articulation brake is positioned at least partially around the second articulation control shaft portion outside of the housing.
23. The endoscope of clause 21 or 22, comprising a first articulation control knob positioned at least partially around the first articulation control shaft portion outside of the housing.
24. The endoscope of any one of clauses 21-23, comprising a second articulation control knob positioned at least partially around the second articulation control shaft portion outside of the housing.
25. The endoscope of clause 23 or clause 24 as dependent from clause 23, wherein the first articulation brake is positioned within the first articulation control knob and between the first articulation control knob and the first articulation control shaft.
26. The endoscope of clause 24 or clause 25 as dependent from clause 24, wherein the second articulation brake is positioned within the second articulation control knob and between the second articulation control knob and the second articulation control shaft.
27. The endoscope of any preceding clause comprising:
   a spring element configurable between a first configuration and a second configuration;
   wherein in the first configuration the spring element forces friction surfaces together so as to resist rotation of an articulation control knob relative to the endoscope housing; and
   wherein in the second configuration the spring is compressed relative to the first configuration and exerts no force or a reduced force upon the friction surfaces such that the brake is disengaged.
28. An endoscope, comprising:
   an articulation control knob and an articulation brake, wherein the articulation brake is configurable from a disengaged configuration that permits rotation of the articulation control knob relative to a housing of the endoscope to an engaged configuration that resists rotation of the articulation control knob relative to the housing;
   wherein the articulation brake includes a spring element and wherein configuring the articulation brake from the engaged configuration to the disengaged configuration compresses the spring element.
29. An endoscope, comprising:
   a reusable hand-piece assembly and a first single-use shaft assembly;
   wherein the reusable hand-piece incorporates electronics for transmitting valve control signals and image data to an external storage device.
30. The endoscope of clause 29, wherein the housing of the reusable hand-piece assembly is arranged for connection to and disconnection from the housing of the first single-use shaft assembly without hand tools.
31. The endoscope of clause 29 or 30, wherein the housing of the reusable hand-piece assembly is removably connected to the housing of the first single-use shaft assembly by one or more latches.
32. The endoscope of clause 29 or 30, wherein the housing of the reusable hand-piece assembly is removably connected to the housing of the first single-use shaft assembly by one or more snap elements.
33. The endoscope of any one of clauses 29-32, further including an optical sensor module.
34. The endoscope of any one of clauses 29-33, wherein both control data and image data are transmitted to a console and an external storage device via a set of electrical conductors associated with the single-use shaft assembly; and
   wherein the control data is transmitted to single-use shaft assembly from the reusable hand-piece via an electrical connector.
35. The endoscope of any one of clauses 29-33, wherein both control data and image data are transmitted to the console and external storage device via a set of electrical conductors associated with the reusable hand-piece; and
   wherein the image data is transmitted to the to the reusable hand-piece via an electrical connector.
36. The endoscope of clauses 34 or 35, wherein three or more segregated switches are associated with the electronics and useful to navigate and select a series of options on a user interface of the console.
37. The endoscope of any one of clauses 34-36, wherein one or more switches are associated with the electronics and useful to initiate capture of images or video or to enhance imaging.
38. The endoscope of any one of clauses 34-37, wherein switches associated with the electronics activate remote valves associated with a console to control fluid flow through the various lumens of the endoscope.
39. The endoscope of clause 33 or any one of clauses 34-38 as dependent from clause 33, wherein the optical sensor module is associated with the single-use shaft assembly and the optical sensor module stores a unique identifier associated with the single-use shaft assembly;
   wherein the unique identifier is communicated to a console that prevents the re-use of a single-use shaft assembly.
40. The system of clause 39, wherein the determining factor that prevents the re-use of a single-use shaft assembly is the repeat electrical connection to a console, exceeded total time of electrical connection to a console, or connection to console during more than one day (24 hour period).
41. The system of clause 39 or 40, wherein the console is connected with a database that shares connectivity data between consoles; and
   wherein the shared data prevents the attempted re-use of a single-use shaft assembly both on a single console and between multiple consoles.
42. The system of any one of clauses 39-41, wherein the re-use of a single-use shaft assembly is prevented by notifying user of issue and not displaying image data on screen or providing any options for storing image data.
43. A system comprising:
   an endoscope and a cartridge;
   the endoscope having a first fluid path, a second fluid path, a third fluid path and an electrical conductor; and
   the first fluid path, second fluid path, third fluid path, and electrical conductor extending from the endoscope to the cartridge;
   the cartridge having a housing and an electrical connector configured to electrically connect the electrical conductor of the endoscope to an electrical conductor of a console;
   wherein the first fluid path includes a first valve portion defined by the cartridge housing, the valve portion aligning with a first actuator of the console when the cartridge is attached to the console, the first actuator actuatable to selectively close the first valve portion of the first fluid path.
44. The system of clause 43, wherein the first valve portion includes a first flexible membrane positioned within a first window defined by the housing, the first window configured to receive the first actuator from the console configured to compress the first flexible membrane to close the first fluid path.
45. The system of clauses 43 or 44 further comprising:

the console.
46. The system of any one of clauses 43-45 wherein the endoscope and cartridge are sterilely sealed within medical packaging.
47. The system of any one of clauses 43-46, comprising a first tube defining the first fluid path, the first tube extending from the endoscope through the cartridge;
   a second tube defining the second fluid, the second tube extending from the endoscope through the cartridge; and
   a third tube defining the third fluid path, the third tube extending from the endoscope through the cartridge.
48. The system of any one of clauses 43-47, wherein the cartridge defines a second valve portion of the second fluid path;
   wherein the second valve portion aligns with a second actuator of the console when the cartridge is attached to the console, the second actuator actuatable to selectively close the second valve portion of the second fluid path.
49. The system of any one of clauses 43-48, wherein the third fluid path has a cartridge portion defined by the cartridge, the cartridge portion having a main portion and a branch portion;
   wherein the main portion of the third fluid path includes a third valve portion;
   wherein the third valve portion aligns with a third actuator of the console, the third actuator actuatable to selectively close the third valve portion;
   wherein the branch portion of the third fluid path includes a fourth valve portion; and
   wherein the fourth valve portion aligns with a fourth actuator of the console, the fourth actuator actuatable to selectively close the fourth valve portion.
50. The system of any one of clauses 43-49, wherein when the cartridge is connected to the console a length of a fourth fluid path extends between the endoscope and the console and is free of a valve.
51. The system of any one of clauses 43-50, wherein when the cartridge is connected to the console a length of the second fluid path extending between the endoscope and the console is free of a valve.
52. The system of any one of clauses 43-51, wherein the endoscope further comprises:
   a reusable hand-piece assembly and a single-use shaft assembly; and
   the reusable hand-piece assembly is selectively attachable to and detachable from the single-use shaft assembly; and
   wherein the reusable hand-piece assembly supports an articulation control and when the
   reusable hand-piece assembly is attached to the single-use shaft assembly the articulation control engages a portion of the single-use shaft assembly for manipulation of a distal part of the single-use shaft assembly.
53. The system of any one of clauses 43-52, wherein the first fluid path extends from a distal tip of the endoscope through the cartridge; and
   wherein a length of the first fluid path from the distal tip of the endoscope to the cartridge is free of a flow controlling valve.
54. The system of clause 45 or any one of clauses 45-53 as dependent from claim 45, wherein the console comprises,
   a door with a latch.
55. The system of clause 54, wherein when the cartridge is attached to the console and the door is closed and latch secured, the door and console compress the cartridge.
56. A method of using the system of any one of clauses 43-55, comprising:
   attaching the cartridge to a console arranged to selectively open and close at least the first
fluid path when the cartridge is attached to the console;
   wherein attaching the cartridge to the console connects the electrical connector of the cartridge to an electrical conductor of the console; and
   wherein the connection of the electrical connector of the cartridge to the electrical conduct of the console allows electrical power to pass between the electrical conductor of the console and the electrical conductor of the endoscope.
57. A cartridge comprising:
   a housing, a first fluid path, a second fluid path and a third fluid path;
   a first electrical connector configured to electrically connect to an electrical conductor of
an endoscope; and
   a second electrical connector configured to electrically connect to an electrical conductor
of a console;
   wherein the first fluid path includes a first valve portion positioned within a first window
defined by an inner surface of the housing;
   wherein the window is configured to receive a first actuator from the console configured
to close the first fluid path.
58. An assembly comprising:
   the cartridge of clause 57, wherein the cartridge is connected to an endoscope comprising:
      a reusable hand-piece assembly and a single-use shaft assembly; and
      the reusable hand-piece assembly selectively attachable to and detachable from
the single-use shaft assembly; and
   wherein the means for articulation is transferred from the reusable hand-piece assembly to the single-use shaft assembly by means of concentric articulation shafts.
59. The system of any previous clause, wherein the cartridge includes an anvil.
60. The system of any previous clause, wherein the cartridge includes a ledge surface and the console includes an opposing ledge surface to receive the ledge surface of the cartridge.
61. The system of any previous clause, wherein the cartridge includes a latch receiving portion and the control valve assembly includes a latch;
   wherein when the cartridge is received by the control valve assembly the latch of the control valve assembly contacts the latch receiving portion of the cartridge.
62. A system comprising:
   a console, a first endoscope, a second endoscope, a first cartridge and a second cartridge;
   the console having a first control valve assembly and a second control valve assembly;
   the first endoscope and the first cartridge defining a first fluid path; and
   the second endoscope and the second cartridge defining a second fluid path; and
   wherein the first control valve assembly receives the first cartridge and the second control valve assembly receives the second cartridge.
63. The system of clause 62, wherein the first endoscope has a control that controls a function of the second control valve assembly when the first cartridge is received in the first control valve assembly.
64. The system of clause 63, wherein:
   the first endoscope comprises a first electrical conductor; and
   the cartridge having a first electrical connector configured to electrically connect the first electrical conductor of the first endoscope to a first electrical conductor of the console.
65. The system of clause 63, wherein the second endoscope and the second cartridge define a suction fluid path, a tissue irrigation fluid path, a camera flush fluid path and an insufflation fluid path, and wherein the second control valve assembly selectively closes one or more of the fluid paths.
66. The system of claim of claim 65, wherein operation of a control of the first endoscope controls the function of one or more of the fluid paths of the second endoscope.
67. The system of clause 62, wherein the second endoscope comprises a light-emitting diode and/or a camera.
68. The system of clause 67, wherein operation of a control of the first endoscope controls the function of the light-emitting diode and/or camera of the second endoscope.
69. The system of clause 62, wherein the first endoscope and the second endoscope are different types of endoscopes.
70. The system of clause 69, wherein the first endoscope is a duodenoscope and the second endoscope is a cholangiopancreatoscope.
71. The system of clause 70, wherein a shaft of the cholangiopancreatoscope extends through a shaft of the duodenoscope.
72. The system of clause 62, wherein a handle of the first endoscope is configured to mate with a handle of the second endoscope.
73. The system of clause 62, wherein a shaft of the second endoscope extends through a shaft of the first endoscope.
74. The system of clause 62, wherein the first endoscope comprises:
   a reusable hand-piece assembly and a single-use shaft assembly; and
   the reusable hand-piece assembly selectively attachable to and detachable from the single-use shaft assembly; and
   wherein the reusable hand-piece assembly includes an articulation control configured to control a distal shaft of the single-use shaft assembly configured for insertion into a patient.
75. The system of clause 62, wherein the first cartridge and the second cartridge are defined by separate cartridge housings.
76. A console comprising:
   a first control valve assembly and a second control valve assembly;
   the first control valve assembly configured to receive a first cartridge; and
   the second control valve assembly configured to receive a second cartridge; and
   wherein the console is configured to receive an electrical signal from the first cartridge in the first control valve assembly and operate an actuator of the second control valve assembly or provide an electrical signal in response thereto.
77. An endoscope comprising:
   a hand-piece assembly and a shaft assembly;
   the hand-piece assembly having a housing;
   the shaft assembly having a housing; and
   a latch configurable between a latched configuration and an unlatched configuration;
   wherein in the latched configuration the latch latches the housing of the hand-piece assembly to the housing of the shaft assembly; and
   wherein when configuring from the latched configuration to the unlatched configuration, the latch applies a separating force to separate the housing of the hand-piece assembly and the housing of the shaft assembly.
78. The endoscope of clause 77 wherein the hand-piece assembly is a reusable hand-piece assembly and the shaft assembly is a single-use shaft assembly.
79. The endoscope of clause 77, wherein the latch comprises a lever and wherein the lever pivots from a first position in the latched configuration to a second position in the unlatched configuration.
80. The endoscope of any preceding clause, wherein the latch has a latching surface and an unlatching surface;
   wherein in the latched configuration the latching surface retains the housing of the hand piece in proximity to the housing of the shaft assembly; and
   wherein when configuring to the unlatched configuration from the latch configuration the unlatching surface applies the separating force to separate the housings.
81. The endoscope of clause 77, wherein the latch further comprises:
   a seat portion.
82. The endoscope of clause 79, wherein the latching portion and unlatching portion are located on opposing sides of the seat portion.
83. The endoscope of clause 77, wherein the latch further comprises:
   a latch pivot.
84. The endoscope of clause 77, wherein the latching portion and the unlatching portion are located on opposing sides of the latch pivot.
85. The endoscope of clause 77, wherein the latch is located on the reusable hand piece.
86. The endoscope of clause, wherein the latch is located on the single-use shaft assembly.
87. An endoscope comprising:
   a hand-piece assembly and a shaft assembly;
   the hand-piece assembly having a housing;
   the shaft assembly having a housing;
   a biasing member; and
   a latch configurable between a latched configuration and an unlatched configuration;
   wherein the biasing member applies a biasing force to bias the housings towards or away from one another; and
   wherein the latch is configured to apply a force counter to the biasing force to force the housings away from or towards one another.
88. The endoscope of clause 87 wherein the biasing member biases the housings towards one another and the latch is configured to force the housings away from one another.
89. The endoscope of clause 87 wherein the biasing member biases the housings away from one another and the latch is configured to force the housings towards one another.
90. The endoscope of any one of clauses 87-89 wherein the biasing member is a spring, a magnet, or a pressure chamber.
91. A latching mechanism for an endoscope having a reusable hand-piece assembly and a single-use shaft assembly comprising:
   a latch and a latch receiving portion;
   the latch having a latching portion, an unlatching portion, and a latch handle; and
   wherein the latching mechanism is movable between a latched configuration and an unlatched configuration;
   wherein the latch handle is movable between a first position and a second position;
   wherein while in the latched configuration the latch receiving portion contacts the latching portion;
   wherein while in the unlatched configuration the latch receiving portion and the latching portion are not in contact; and
   wherein when configuring from the latched configuration to the unlatched configuration, the unlatching portion contacts the receiving portion and applies a force.
92. A packaging system for a medical product comprising:
   a medical product box;
   a disposal bag and a container both removably positioned within said medical product box prior to use of the medical product;
   said container including:
      a first portion including a recess retaining the medical productin a sterile environment; and
   wherein said disposal bag is sized to retain at least a disposable portion and/or reusable portion of said medical product after said medical product has been used.
93. The packaging system of clause 92, wherein said container includes a tray and wherein said recess retaining said medical product is defined within said tray.
94. The packaging system of clause 93, wherein said tray is fabricated from thermoformed plastic.
95. The packaging system of any one of clauses 92-94, wherein said container includes a is cover sterilely sealing the medical product in said recess.
96. The packaging system of clause 95, wherein said cover is made from Tyvek®.
97. The packaging system of any one of clauses 95-96, further comprising:
   a label adhered to said cover, wherein said label includes information pertaining to said medical product retained within said recess.
98. The packaging system of any one of clauses 92-97, wherein said disposal bag is secured to a second portion of said container when said container and said disposal bag are within said medical product box.
99. The packaging system of clause 98, wherein said second portion is an exterior surface of said container.
100. The packaging system of clause 98, wherein said second portion is an interior surface of said container.
101. The packaging system of any one of clauses 98-100, wherein said disposal bag is detachable from said second portion of said container.
102. The packaging system of any one of clauses 92-101, wherein said container is made from a recyclable material.
103. The packaging system of any one of clauses 92-102, further comprising:
   a label adhered to said medical product box, wherein said label includes information pertaining to said medical product.
104. The packaging system of any one of clauses 92-103, further comprising:
   a shipping box; and
   wherein multiple of said medical product boxes are packed within said shipping box.
105. The packaging system of any one of clauses 92-104, wherein said medical product is an endoscope.
106. The packaging system of any one of clauses 98-105, where said disposal bag includes a perimeter opening, and wherein the disposal bag is secured to said second portion of said container along at least 20% of said perimeter opening.
107. The packaging system of any one of clauses 98-106, wherein said disposal bag is adhered to said second portion of said container.
108. The packaging system of clause 107, wherein adhesive for adhering the disposal bag to said second portion of said container is spaced along at least 20% of said perimeter opening.
109. An endoscope, comprising:
   a reusable hand-piece assembly and a first single-use shaft assembly releasably coupled to said reusable hand-piece assembly;
   wherein the reusable hand-piece assembly includes one or more buttons for controlling operation of the endoscope, the operation including at least one of aspiration and insufflation;
   wherein one or more of the buttons include a capacitive sensor.
110. A method, comprising:
   the endoscope of clause 109 wherein a capacitive sensor is utilized to actuate a valve external to the reusable hand-piece assembly and first single-use shaft assembly of the endoscope.
111. A separable medical device, comprising:
   a reusable hand-piece assembly and a single-use shaft assembly;
   the reusable hand-piece assembly selectively attachable to and detachable from the to single-use shaft assembly;
   the reusable hand-piece assembly including a housing, a first articulation control knob, and a first shaft having a first end coupled the first articulation knob such that rotation of the first articulation knob rotates the first shaft; and
   the single-use shaft assembly including a housing and an endoscope insertion tube;
   wherein the housing of the single-use shaft assembly contains a first pulley and associated set of articulation wire segments that are fixed at the distal tip of the articulating section of the endoscope insertion tube;
   wherein the first shaft has a second end having a plurality of torque transmission bosses spaced around the periphery thereof and that engage with torque transmission bosses of the first pulley when the reusable hand-piece assembly is coupled to the single-use shaft assembly so that the first articulation control knob controls the rotation of the first pulley via the first shaft; and
   wherein the torque transmission bosses of the reusable hand-piece assembly and/or single-use shaft assembly include alignment ramps on ends of the torque transmission bosses facing the opposing housing when the reusable hand-piece assembly and single-use shaft assembly are coupled.
112. The separable medical device of clause 111, wherein said torque transmission bosses have symmetrical alignment ramps on said ends.
113. The separable medical device of any one of clauses 111 or 112, wherein the hand-piece assembly includes a first articulation brake and an articulation brake shaft concentrically located within the first articulation shaft and that engages a socket of the single-use shaft housing;; and
   wherein the articulation brake shaft is rotationally fixed relative to the socket via a series of torque transmission bosses having alignment ramps on at least one end thereof.
114. The endoscope of any one of clauses 1-42 having torque transmission bosses associated to with any or all of the articulation controls and/or articulation brakes.
115. A method of fabricating at least a portion of a flexible endoscope shaft, comprising:
   forming a continuous first length, second length, and third length of outer coil using a continuous wire coiler, wherein forming the first length defines a first period of time, forming the second length defines a second period of time, and forming the third length defines a third period of time; and
   forming an outer braid around said outer coil along the first length during said second period of time to make a braided assembly.
116. The method of clause 115, comprising:
   positioning within said outer coil two or more pull wires, each pull wire located within a concentric compression coil.
117. A method of any one of clauses 115-116, wherein the flexible endoscope shaft comprises a distal articulating section extending distally of the proximal insertion tube.
118. The method of any one of clauses 115-117, wherein the outer braid is formed with discrete wires.
119. The method of any one of clauses 115-118, comprising advancing the braided assembly through an opening in a die of an extrusion mold during said third period of time.
120. The method of clause 119, comprising forming an outer braid around said outer coil along the second length during said third period of time.
121. The method of clauses 119 or 120 further comprising applying resins of different durometers along said first length.
122. The method of any one of clauses 115-121 comprising: applying an identifier to the assembly identifying a cut location.
123. The method of clause 122 as dependent from clause 121, wherein a length of the assembly having resins of different durometers is free of the identifier.
124. An articulating joint for a medical device, comprising:
   a plurality of articulating links that incorporate concentric tab and socket pivot joints having tabs extending from each link and engaging sockets of an adjacent link;
   a proximal link incorporating retention cavities for compression coils;
   a distal link incorporating a keyed profile for orienting a distal cap; and
   distal cap incorporating corresponding keyed profile for engaging the distal link and to allowing only one rotational orientation between the distal cap and the distal link.
125. An articulating joint for a medical device, comprising:
   a plurality of articulating links that incorporate concentric tab and socket pivot joints having tabs extending from each link and engaging sockets of an adjacent link;
   a looped steering wire that radially wraps 180 degrees or some multiple of 180 degrees around the circumference of the distal link; and
   wherein the looped steering wire is fixedly coupled to the distal link.
126. An articulating joint for a medical device, comprising:
   a plurality of articulating links that incorporate concentric tab and socket pivot joints having tabs extending from each link and engaging sockets of an adjacent link;
   a pair of looped steering wires that radially wrap 90 degrees or some multiple of 90 degrees around the circumference of the distal link; and
   wherein the looped steering wire is fixedly coupled to the distal link.
127. An articulating joint for a medical device, comprising:
   a plurality of articulating links that incorporate concentric tab and socket pivot joints having tabs extending from each link and engaging sockets of an adjacent link;
   one or more breakaway struts attached to the periphery of individual links of the plurality of links by one or more distinct connection points;
   a proximal link; and
   a distal link.
128. An articulating joint for a medical device, comprising:
   a plurality of articulating links that incorporate concentric tab and socket pivot joints having tabs extending from each link and engaging sockets of an adjacent link;
   a proximal link with one or more channels;
   a distal link with one or more channels; and
   one or more breakaway struts attached to the periphery of a proximal link, the individual links of the plurality of links, and a distal link by one or more distinct connection points.
129. The articulating joint of any one of clauses 127-128, wherein concentric tabs and sockets of each link of the plurality of links are arranged orthogonally to one another.
130. The articulating joint of any one of clauses 127-129, wherein the anterior face of the proximal link interfaces with a posterior face of the adjacent link in the plurality of links which allows for articulation between the links.
131. The articulating joint of clause 130, wherein the proximal link includes fingers that engage the interior of an insertion tube for connection with a shaft.
132. The articulating joint of any one of clauses 127-131, wherein the distal link includes a keyed profile that engage a distal cap, a posterior face interfacing the anterior face of an adjacent link in the plurality of links, and/or a cylindrical shape.
133. The articulating joint of clause 132, wherein the posterior face interfaces with the anterior face of the anterior link of the plurality of links and allows for articulation between the links.
134. The articulating joint of any one of clauses 127-133, wherein the proximal link includes fingers that engage the interior of an insertion tube for connection with a shaft.
135. The articulating joint of any one of clauses 127-134, wherein a proximal link, a plurality of articulating links, and a distal link include one or more lumens.

### GLOSSARY OF TERMS

The language used in the claims and the written description is to only have its plain and ordinary meaning, except for terms explicitly defined below. Such plain and ordinary is meaning is defined here as inclusive of all consistent dictionary definitions from the most recently published (on the filing date of this document) general purpose Merriam-Webster dictionary.

As used in the claims and the specification, the following terms have the following defined meanings:

“Actuators” as used herein include linear actuators and rotary actuators. A solenoid is one example of an actuator.

“Distal” generally refers to the opposite end of proximal (“patient end/treating end”).

“Electrical connector” generally refers to an electromechanical device used to join electrical terminations and create an electrical circuit. Electrical connectors may have a gender—i.e., the male component, called a plug, which connects to the female component, or socket. The connection may be removable.

“Electrical signal” generally refers to a voltage or current which conveys information. It may also include passing electric power from one component to another.

“Proximal” generally refers to an end or direction associated with a physician, other treating personnel during a device operation, or otherwise connected to an insertion tube.

The term “pulley” as used herein can include cams.

The term “valve” as used herein may include mechanical devices by which the flow of liquid, gas, or loose material in bulk may be started, stopped, or regulated by a movable part that opens, shuts, or partially obstructs one or more ports or passageways, such as butterfly valves, needle valves, ball valves, or pinch valves, just to name a few non-limiting examples.

1. An endoscope, comprising:
a reusable hand-piece assembly and a single-use shaft assembly;
the reusable hand-piece assembly selectively attachable to and detachable from the single-use shaft assembly;
the reusable hand-piece assembly including a housing, a first articulation control knob, a second articulation control knob, a first articulation brake, and a second articulation brake; and
the single-use shaft assembly including a housing and an endoscope shaft;
wherein the first articulation brake is configurable from a disengaged position that permits rotation of the first articulation control knob relative to the housing of the reusable hand-piece to an engaged position that resists rotation of the first articulation control knob relative to the housing of the reusable hand-piece; and
wherein the second articulation brake is configurable from a disengaged position that is permits rotation of the second articulation control knob relative to the housing of the single-use shaft assembly to an engaged position that resists rotation of the second articulation control knob relative to the housing of the single-use shaft assembly when the reusable hand-piece assembly is attached to the single-use shaft assembly.

2. The endoscope of claim 1, wherein the second articulation brake includes a brake articulation shaft extending through the first articulation control knob.

3. The endoscope of claim 2, wherein when the reusable hand-piece assembly is engaged to the single-use assembly an end of the brake articulation shaft engages the housing of the single-use shaft assembly so as to rotationally fix the brake articulation shaft relative to the single-use shaft assembly.

4. The endoscope of claim 2, wherein the brake articulation shaft extends through a first articulation control shaft associated with the first articulation control knob; and
wherein the first articulation control shaft rotates relative to the housing of the reusable hand-piece assembly upon rotation of the first articulation control knob relative to the housing of the reusable hand-piece assembly.

5. The endoscope of claim 4, wherein a second articulation control shaft associated with the second articulation control knob extends through the first articulation control shaft; and
wherein the second articulation control shaft rotates relative to the housing of the reusable hand-piece assembly upon rotation of the second articulation control knob relative to the housing of the reusable hand-piece assembly.

6. The endoscope of claim 5, wherein the brake articulation shaft extends through the second articulation control shaft.

7. The endoscope of claim 4, wherein the first articulation brake includes an outer brake housing and a caliper;
wherein the outer brake housing is associated with the first articulation control knob and first articulation control shaft such that rotation of the first articulation control knob rotates the outer brake housing and the first articulation control shaft;
wherein the caliper is rotationally fixed relative to the reusable hand-piece; and
wherein the caliper moves towards the outer brake housing when the first articulation brake is configured from the disengaged configuration to the engaged configuration.

8. The endoscope of claim 7, wherein the first articulation brake includes an inner brake housing positioned within the outer brake housing; and
wherein the inner brake housing rotates relative to the caliper so as to translate the caliper towards the outer brake housing when the first articulation brake is configured from the disengaged configuration to the engaged configuration.

9. The endoscope of claim 8, wherein the inner brake housing is rotationally fixed to a brake control lever.

10. The endoscope of claim 8, wherein the outer brake housing, the inner brake housing, and the caliper are all positioned within the first articulation control knob.

11. The endoscope of claim 5, wherein the second articulation brake includes an outer brake housing and a caliper;
wherein the outer brake housing is associated with the second articulation control knob and second articulation control shaft such that rotation of the second articulation control knob rotates the outer brake housing and the second articulation control shaft;
wherein the caliper is rotationally fixed relative to the brake articulation shaft; and
wherein the caliper moves towards the outer brake housing when the second articulation brake is configured from the disengaged configuration to the engaged configuration.

12. The endoscope of claim 11, wherein the second articulation brake includes an inner brake housing positioned within the outer brake housing; and
wherein the inner brake housing rotates relative to the caliper so as to translate the caliper towards the outer brake housing when the second articulation brake is configured from the disengaged configuration to the engaged configuration.

13. The endoscope of claim 12, wherein the inner brake housing is rotationally fixed to a brake control knob.

14. The endoscope of claim 12, wherein the outer brake housing, the inner brake housing, and the caliper are all positioned within the second articulation control knob.

15. The endoscope of claim 1, wherein the first articulation control knob is positioned between the housing of the reusable hand-piece and the second articulation control knob.

16. An endoscope, comprising:
a hand-piece assembly including a housing, a first articulation control knob, a second articulation control knob, a first articulation control shaft, a second articulation control shaft, a first articulation brake, and a second articulation brake;
a first articulation control shaft associated with the first articulation control knob;
wherein the first articulation control shaft rotates relative to the housing upon rotation of the first articulation control knob relative to the housing;
wherein the second articulation control shaft rotates relative to the housing upon rotation of the second articulation control knob relative to the housing;
wherein the first articulation brake is configurable from a disengaged position that permits rotation of the first articulation control shaft relative to the housing to an engaged position that resists rotation of the first articulation control shaft relative to the housing;
wherein the second articulation brake is configurable from a disengaged position that permits rotation of the second articulation control shaft relative to the housing to an engaged position that resists rotation of the second articulation control shaft relative to the housing; and
wherein the second articulation brake includes a brake articulation shaft extending through the second articulation control shaft.

17. The endoscope of claim 16, wherein the brake articulation shaft is rotationally fixed relative to the housing.

18. The endoscope of claim 16, wherein the second articulation control shaft extends is through the first articulation control shaft.

19. The endoscope of claim 16, wherein the first articulation control shaft extends through a steering knob hub that is rotationally fixed relative to the housing.

20. The endoscope of claim 19, wherein the first articulation control knob is rotationally fixed relative to the steering knob hub when the first articulation brake is configured in the engaged position.
